(19) United States
(12) Patent Application Publication
(10) Pub. No.: US 2019/0330394 A1
(43) Pub. Date: Oct. 31, 2019

(54) Alkyl Ammonium (Fluoroaryl)borate Activators

(71) Applicant: ExxonMobil Chemical Patents Inc., Baytown, TX (US)

(72) Inventors: Catherine A. Faler, Houston, TX (US); Margaret T. Whalley, Houston, TX (US); Crisita Carmen H. Atienza, Houston, TX (US); John R. Hagadorn, Houston, TX (US)

(21) Appl. No.: 16/394,174

(22) Filed: Apr. 25, 2019

Related U.S. Application Data
(60) Provisional application No. 62/662,981, filed on Apr. 26, 2018.

(51) Int. Cl. C08F 110/06; C08F 210/16; C07F 5/02; C07C 211/64

(52) U.S. Cl. CPC C08F 110/06; C07C 211/64; C07F 5/027; C08F 210/16

(57) ABSTRACT

The present disclosure provides borate activators, catalyst systems comprising borate activators, and methods for polymerizing olefins using borate activators. The borate activator compounds are represented by Formula (I): [R¹R²R³EH]⁺[BR⁴R⁵R⁶R⁷]⁻, wherein: E is nitrogen or phosphorous; R¹ is an electron deficient aromatic group; each of R² and R³ is independently C₁-C₄₀ alkyl, C₅-C₂₂-aryl, wherein each of R¹, R², and R³ is independently unsubstituted or substituted with at least one of halide, C₁-C₁₀ alkyl, C₅-C₁₅ aryl, C₆-C₂₅ arylalkyl, and C₆-C₂₅ alkylaryl, wherein R¹, R², and R³ together comprise 15 or more carbon atoms; and each of R⁴, R⁵, R⁶, and R⁷ is aryl (such as phenyl or naphthyl), wherein at least one of R⁴, R⁵, R⁶, and R⁷ is substituted with one or more fluorine atoms.

### PRIORITY CLAIM

This application claims priority to and the benefit of U.S. Ser. No. 62/662,981, filed Apr. 26, 2018 and is incorporated by reference in its entirety.

### CROSS REFERENCE TO RELATED APPLICATIONS

This application is related to concurrently filed application U.S. Ser. No. 62/662,972, filed Apr. 26, 2018 and U.S. Ser. No. 62/769,208, filed Nov. 19, 2018.

### FIELD

The present disclosure provides ammonium borate activators, catalyst systems comprising ammonium borate activators, and methods for polymerizing olefins using ammonium borate activators.

### BACKGROUND

Polyolefins are widely used commercially because of their robust physical properties. For example, various types of polypropylene are some of the most commercially useful. Polyolefins are typically prepared with a catalyst that polymerizes olefin monomers. Therefore, there is interest in finding new catalysts and catalyst systems that provide polymers having improved properties.

Catalysts for olefin polymerization are based on metallocenes as catalyst precursors, which are activated either with an alumoxane or an activator containing a non-coordinating anion. A non-coordinating anion, such as tetrakis(pentafluorophenyl)borate, is capable of stabilizing the resulting Group 4 metal cation of the catalyst. Because such activators are fully ionized and the corresponding anion is highly non-coordinating, such activators can be effective as olefin polymerization catalyst activators. However, because they are ionic salts, such activators are insoluble in aliphatic hydrocarbons and only sparingly soluble in aromatic hydrocarbons. It is desirable to conduct most polymerizations of α-olefins in aliphatic hydrocarbon solvents due to the compatibility of such solvents with the olefin monomer and in order to reduce the aromatic hydrocarbon content of the resulting polymer product. Typically, ionic salt activators are added to such polymerizations in the form of a solution in an aromatic solvent such as toluene. The use of even a small quantity of such an aromatic solvent for this purpose is undesirable since it must be removed in a post-polymerization devolatilization step and separated from other volatile components, which is a process that adds significant cost and complexity to any commercial process. In addition, the activators often exist in the form of an oily, intractable material which is not readily handled and metered or precisely incorporated into the reaction mixture.

In addition, polymer products, such as isotactic polypropylene, formed using such activators do not have a high molecular weight (e.g., Mw greater than about 100,000) and a high melt temperature (Tm) (e.g., Tm greater than about 110° C.).

There is a need for activators that are soluble in aliphatic hydrocarbons and capable of producing polyolefins having a high molecular weight and high melt temperature.

References of interest include: U.S. Pat. Nos. 7,799,879; 7,985,816; 8,580,902; 8,835,587; WO2010/014344; U.S. Pat. Nos. 8,642,497; 5,919,983; 6,121,185; WO 2002/002577; U.S. Pat. Nos. 7,087,602; 8,642,497; 6,121,185; US 2015/0203602; CAS number 909721-53-5, CAS number 943521-08-2; US 2002/0062011; and US 2003/0013913.

### SUMMARY

The present disclosure relates to activator compounds represented by Formula (AI):

[R¹R²R³EH^{d+}][M^{k+}Qₙ]^{d-} (AI)

wherein:
E is nitrogen or phosphorous;
R¹ is an electron deficient aromatic group;
each of R² and R³ is independently C₁-C₄₀ alkyl, C₅-C₂₂-aryl, wherein each of R¹, R², and R³ is independently unsubstituted or substituted with at least one of halide, C₁-C₁₀ alkyl, C₅-C₁₅ aryl, C₆-C₂₅ arylalkyl, and C₆-C₂₅ alkylaryl, wherein R¹, R², and R³ together comprise 15 or more carbon atoms;
d is 1, 2, or 3; k is 1, 2, or 3; n is 1, 2, 3, 4, 5, or 6; n−k=d (preferably d is 1, 2 or 3; k is 3; n is 4, 5, or 6);
M is an element selected from group 13 of the Periodic Table of the Elements; and
each Q is independently a hydride, bridged or unbridged dialkylamido, halide, alkoxide, aryloxide, hydrocarbyl, substituted hydrocarbyl, halocarbyl, substituted halocarbyl, or halosubstituted-hydrocarbyl radical.

The present disclosure relates to activator compounds represented by Formula (I):

[R¹R²R³EH]⁺[BR⁴R⁵R⁶R⁷]⁻ (I)

wherein:
E is nitrogen or phosphorous;
R¹ is an electron deficient aromatic group;
each of R² and R³ is independently C₁-C₄₀ alkyl, C₅-C₂₂-aryl, wherein each of R¹, R², and R³ is independently unsubstituted or substituted with at least one of halide, C₁-C₁₀ alkyl, C₅-C₁₅ aryl, C₆-C₂₅ arylalkyl, and C₆-C₂₅ alkylaryl, wherein R¹, R², and R³ together comprise 15 or more carbon atoms; and
each of R⁴, R⁵, R⁶, and R⁷ is aryl (such as phenyl or naphthyl), wherein at least one of R⁴, R⁵, R⁶, and R⁷ is substituted with one or more fluorine atoms.

In yet another embodiment, the present disclosure provides a catalyst system comprising an activator of the present disclosure and a catalyst.

In yet another embodiment, the present disclosure provides a catalyst system comprising an activator of the present disclosure, a catalyst support, and a catalyst.

In still another embodiment, the present disclosure provides a polymerization process comprising a) contacting one or more olefin monomers with a catalyst system comprising: i) an activator of the present disclosure and ii) a catalyst,

In still another embodiment, the present disclosure provides a polyolefin formed by a catalyst system and or method of the present disclosure.

In yet another embodiment, the present disclosure provides a catalyst system comprising an activator of the present disclosure and a catalyst, absent toluene.

### DETAILED DESCRIPTION

The present disclosure relates to activator compounds that can be used in olefin polymerization processes. For example, the present disclosure provides ammonium borate activators, catalyst systems comprising ammonium borate activators, and methods for polymerizing olefins using ammonium borate activators. In the present disclosure, ammonium borate activators are described that feature ammonium groups with long-chain aliphatic hydrocarbyl groups for improved solubility of the activator in aliphatic solvents, as compared to conventional activator compounds. Borate groups of the present disclosure are fluoronaphthyl borates. It has been discovered that activators of the present disclosure having fluoronaphthyl borates have improved solubility in aliphatic solvents, as compared to conventional activator compounds. Activators of the present disclosure can provide polyolefins having a weight average molecular weight (Mw) of about 100,000 g/mol or greater and a melt temperature (Tm) of about 110° C. or greater.

In another aspect, the present disclosure relates to polymer compositions obtained from the catalysts systems and processes set forth herein. The components of the catalyst systems according to the present disclosure and used in the polymerization processes of the present disclosure, as well as the resulting polymers, are described in more detail herein below.

The present disclosure relates to a catalyst system comprising a transition metal compound and an activator compound of formula (I), to the use of an activator compound of formula (I) for activating a transition metal compound in a catalyst system for polymerizing olefins, and to processes for polymerizing olefins, the process comprising contacting under polymerization conditions one or more olefins with a catalyst system comprising a transition metal compound and an activator compound of formula (I).

The present disclosure also relates to processes for polymerizing olefins comprising contacting, under polymerization conditions, one or more olefins with a catalyst system comprising a transition metal compound and an activator compound of formula (I). The weight average molecular weight of the polymer formed can increase with increasing monomer conversion at a given reaction temperature.

The present disclosure relates to a catalyst system comprising a transition metal compound and an activator compound of formula (I) of (AI), to the use of an activator compound of formula (I) or (AI) for activating a transition metal compound in a catalyst system for polymerizing olefins, and to processes for polymerizing olefins, the process comprising contacting under polymerization conditions one or more olefins with a catalyst system comprising a transition metal compound and an activator compound of formula (I) or (AI), where aromatic solvents, such as toluene, are absent (e.g. present at zero mol %, alternately present at less than 1 mol %, preferably the catalyst system, the polymerization reaction and/or the polymer produced are free of “detectable aromatic hydrocarbon solvent,” such as toluene. For purposes of the present disclosure, “detectable aromatic hydrocarbon solvent” means 0.1 mg/m² or more as determined by gas phase chromatography. For purposes of the present disclosure, “detectable toluene” means 0.1 mg/m² or more as determined by gas phase chromatography.

The polyolefins produced herein preferably contain 0 ppm of aromatic hydrocarbon. Preferably, the polyolefins produced herein contain 0 ppm of toluene.

The catalyst systems used herein preferably contain 0 ppm of aromatic hydrocarbon. Preferably, the catalyst systems used herein contain 0 ppm of toluene.

The activator compound of formula (I) and (IA) will be further illustrated below. Any combinations of cations and NCAs disclosed herein are suitable to be used in the processes of the present disclosure and are thus incorporated herein.

Unless otherwise noted all melt temperatures (Tm) are DSC second melt and are determined using the following DSC procedure according to ASTM D3418-03. Differential scanning calorimetric (DSC) data are obtained using a TA Instruments model Q200 machine. Samples weighing about 5 to about 10 mg are sealed in an aluminum hermetic sample pan. The DSC data are recorded by first gradually heating the sample to about 200° C. at a rate of about 10° C./minute. The sample is kept at about 200° C. for about 2 minutes, then cooled to about −90° C. at a rate of about 10° C./minute, followed by an isothermal for about 2 minutes and heating to about 200° C. at about 10° C./minute. Both the first and second cycle thermal events are recorded. The melting points reported herein are obtained during the second heating/cooling cycle unless otherwise noted.

All molecular weights are weight average (Mw) unless otherwise noted. All molecular weights are reported in g/mol unless otherwise noted. Melt index (MI) also referred to as 12, reported in g/10 min, is determined according to ASTM D-1238, 190° C., 2.16 kg load. High load melt index (HLMI) also referred to as 121, reported in g/10 min, is determined according to ASTM D-1238, 190° C., 21.6 kg load. Melt index ratio (MIR) is MI divided by HLMI as determined by ASTM D1238.

The specification describes catalysts that can be transition metal complexes. The term complex is used to describe molecules in which an ancillary ligand is coordinated to a central transition metal atom. The ligand is bulky and stably bonded to the transition metal so as to maintain its influence during use of the catalyst, such as polymerization. The ligand may be coordinated to the transition metal by covalent bond and/or electron donation coordination or intermediate bonds. The transition metal complexes are generally subjected to activation to perform their polymerization or oligomerization function using an activator which is believed to create a cation as a result of the removal of an anionic group, often referred to as a leaving group, from the transition metal.

For the purposes of the present disclosure, the numbering scheme for the Periodic Table Groups is used as described in Chemical and Engineering News, 63(5), pg. 27 (1985). Therefore, a “Group 8 metal” is an element from Group 8 of the Periodic Table, e.g., Fe.

The following abbreviations are used through this specification: o-biphenyl is an ortho-biphenyl moiety represented by the structure,

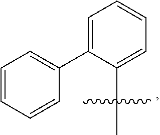 [SMILES: *c1ccccc1-c1ccccc1]

dme is 1,2-dimethoxyethane, Me is methyl, Ph is phenyl, Et is ethyl, Pr is propyl, iPr is isopropyl, n-Pr is normal propyl, cPr is cyclopropyl, Bu is butyl, iBu is isobutyl, tBu is tertiary butyl, p-tBu is para-tertiary butyl, nBu is normal butyl, sBu is sec-butyl, TMS is trimethylsilyl, TIBAL is triisobutylaluminum, TNOAL is tri(n-octyl)aluminum, MAO is methylalumoxane, p-Me is para-methyl, Ph is phenyl, Bn is benzyl (i.e., CH₂Ph), THF (also referred to as thf) is tetrahydrofuran, RT is room temperature (and is 23° C. unless otherwise indicated), tol is toluene, EtOAc is ethyl acetate, and Cy is cyclohexyl.

Unless otherwise indicated (e.g., the definition of “substituted hydrocarbyl”, etc.), the term “substituted” means that at least one hydrogen atom has been replaced with at least a non-hydrogen group, such as a hydrocarbyl group, a heteroatom, or a heteroatom containing group, such as halogen (such as Br, Cl, F or I) or at least one functional group such as —NR*₂, —OR*, —SeR*, —TeR*, —PR*₂, —AsR*₂, —SbR*₂, —SR*, —BR*₂, —SIR*, —SiR*₃, —GeR*, —GeR*₃, —SnR*, —SnR*₃, —PbR*₃, and the like, where each R* is independently a hydrocarbyl or halocarbyl radical, and two or more R* may join together to form a substituted or unsubstituted saturated, partially unsaturated or aromatic cyclic or polycyclic ring structure, or where at least one heteroatom has been inserted within a hydrocarbyl ring.

The terms “hydrocarbyl radical,” “hydrocarbyl,” and “hydrocarbyl group,” are used interchangeably throughout this disclosure. Likewise, the terms “group”, “radical”, and “substituent” are also used interchangeably in this disclosure. For purposes of this disclosure, “hydrocarbyl radical” is defined to be C₁-C₁₀₀ radicals, that may be linear, branched, or cyclic, and when cyclic, aromatic or non-aromatic. Examples of such radicals can include methyl, ethyl, n-propyl, isopropyl, n-butyl, isobutyl, sec-butyl, tert-butyl, pentyl, iso-amyl, hexyl, octyl cyclopropyl, cyclobutyl, cyclopentyl, cyclohexyl, cyclooctyl, and the like including their substituted analogues. Substituted hydrocarbyl radicals are radicals in which at least one hydrogen atom of the hydrocarbyl radical has been substituted with at least a non-hydrogen group, such as a hydrocarbyl group, a heteroatom, or a heteroatom containing group, such as halogen (such as Br, Cl, F, or I) or at least one functional group such as —NR*₂, —OR*, —SeR*, —TeR*, —PR*₂, —AsR*₂, —SbR*₂, —SR*, —BR*₂, —SIR*, —SiR*₃, —GeR*, —GeR*₃, —SnR*, —SnR*₃, —PbR*₃, and the like, or where at least one heteroatom has been inserted within a hydrocarbyl ring.

The terms “alkyl radical,” and “alkyl” are used interchangeably throughout this disclosure. For purposes of this disclosure, “alkyl radical” is defined to be C₁-C₁₀₀ alkyls that may be linear, branched, or cyclic. Examples of such radicals can include methyl, ethyl, n-propyl, isopropyl, n-butyl, isobutyl, sec-butyl, tert-butyl, pentyl, iso-amyl, hexyl, octyl cyclopropyl, cyclobutyl, cyclopentyl, cyclohexyl, cyclooctyl, and the like including their substituted analogues. Substituted alkyl radicals are radicals in which at least one hydrogen atom of the alkyl radical has been substituted with at least a non-hydrogen group, such as a hydrocarbyl group, a heteroatom, or a heteroatom containing group, such as halogen (such as Br, Cl, F, or I) or at least one functional group such as —NR*₂, —OR*, —SeR*, —TeR*, —PR*₂, —AsR*₂, —SbR*₂, —SR*, —BR*₂, —SIR*, —SiR*₃, —GeR*, —GeR*₃, —SnR*, —SnR*₃, —PbR*₃, and the like, or where at least one heteroatom has been inserted within a hydrocarbyl ring.

The term “alkenyl” means a straight-chain, branched-chain, or cyclic hydrocarbon radical having one or more carbon-carbon double bonds. These alkenyl radicals may be substituted. Examples of suitable alkenyl radicals can include ethenyl, propenyl, allyl, 1,4-butadienyl cyclopropenyl, cyclobutenyl, cyclopentenyl, cyclohexenyl, cyclooctenyl and the like including their substituted analogues.

The term “arylalkenyl” means an aryl group where a hydrogen has been replaced with an alkenyl or substituted alkenyl group. For example, styryl indenyl is an indene substituted with an arylalkenyl group (a styrene group).

The term “alkoxy”, “alkoxyl”, or “alkoxide” means an alkyl ether or aryl ether radical wherein the term alkyl is as defined above. Examples of suitable alkyl ether radicals can include methoxy, ethoxy, n-propoxy, iso-propoxy, n-butoxy, iso-butoxy, sec-butoxy, tert-butoxy, phenoxy, and the like.

The term “aryl” or “aryl group” means a carbon-containing aromatic ring and the substituted variants thereof can include phenyl, 2-methyl-phenyl, xylyl, 4-bromo-xylyl. Likewise, heteroaryl means an aryl group where a ring carbon atom (or two or three ring carbon atoms) has been replaced with a heteroatom, such as N, O, or S. As used herein, the term “aromatic” also refers to pseudoaromatic heterocycles which are heterocyclic substituents that have similar properties and structures (nearly planar) to aromatic heterocyclic ligands, but are not by definition aromatic; likewise, the term aromatic also refers to substituted aromatics.

The term “arylalkyl” means an aryl group where a hydrogen has been replaced with an alkyl or substituted alkyl group. For example, 3,5′-di-tert-butyl-phenyl indenyl is an indene substituted with an arylalkyl group.

The term “alkylaryl” means an alkyl group where a hydrogen has been replaced with an aryl or substituted aryl group. For example, phenethyl indenyl is an indene substituted with an ethyl group bound to a benzene group.

The term “haloalkyl” means an alkyl group where a hydrogen has been replaced with halogen, wherein the term alkyl is as defined above and halogen is a group 17 element.

The term “electron deficient aromatic group” is defined to be an aryl group substituted with one or more halogen or haloalkyl groups. Preferably an electron deficient aromatic group is a phenyl group substituted with one, two, three, four or five halogen or haloalkyl groups, or a naphthyl group substituted with one, two, three, four, five, six or seven halogen or haloalkyl groups.

Reference to an alkyl, alkenyl, alkoxide, or aryl group without specifying a particular isomer (e.g., butyl) expressly discloses all isomers (e.g., n-butyl, iso-butyl, sec-butyl, and tert-butyl), unless otherwise indicated.

The term “ring atom” means an atom that is part of a cyclic ring structure. Accordingly, a benzyl group has six ring atoms and tetrahydrofuran has 5 ring atoms.

A heterocyclic ring is a ring having a heteroatom in the ring structure as opposed to a heteroatom substituted ring where a hydrogen on a ring atom is replaced with a heteroatom. For example, tetrahydrofuran is a heterocyclic ring and 4-N,N-dimethylamino-phenyl is a heteroatom substituted ring.

For purposes of the present disclosure, a “catalyst system” is a combination of at least one catalyst compound, an activator, and an optional support material. The catalyst systems may further comprise one or more additional catalyst compounds. The terms “mixed catalyst system”, “dual catalyst system”, “mixed catalyst”, and “supported catalyst system” may be used interchangeably herein with “catalyst system.” For the purposes of the present disclosure, when catalyst systems are described as comprising neutral stable forms of the components, it is well understood by one of ordinary skill in the art, that the ionic form of the component is the form that reacts with the monomers to produce polymers. Catalysts of the presented disclosure and activators represented by Formula (I) are intended to embrace ionic forms in addition to the neutral forms of the compounds.

“Complex” as used herein, is also often referred to as catalyst precursor, precatalyst, catalyst, catalyst compound, transition metal compound, or transition metal complex. These words are used interchangeably. Activator and cocatalyst are also used interchangeably.

A scavenger is a compound that is typically added to facilitate polymerization by scavenging impurities. Some scavengers may also act as activators and may be referred to as co-activators. A co-activator, that is not a scavenger, may also be used in conjunction with an activator in order to form an active catalyst. In some embodiments a co-activator can be pre-mixed with the transition metal compound to form an alkylated transition metal compound.

Noncoordinating anion (NCA) means an anion either that does not coordinate to the catalyst metal cation or that does coordinate to the metal cation, but only weakly. The term NCA is also defined to include multicomponent NCA-containing activators, such as N,N-dimethylanilinium tetrakis(pentafluorophenyl)borate, that contain an acidic cationic group and the non-coordinating anion. The term NCA is also defined to include neutral Lewis acids, such as tris(pentafluorophenyl)boron, that can react with a catalyst to form an activated species by abstraction of an anionic group. An NCA coordinates weakly enough that a neutral Lewis base, such as an olefinically or acetylenically unsaturated monomer can displace it from the catalyst center. Any metal or metalloid that can form a compatible, weakly coordinating complex may be used or contained in the noncoordinating anion. Suitable metalloids can include boron, aluminum, phosphorus, and silicon. The term non-coordinating anion activator includes neutral activators, ionic activators, and Lewis acid activators.

In the description herein, a metallocene catalyst may be described as a catalyst precursor, a pre-catalyst compound, metallocene catalyst compound or a transition metal compound, and these terms are used interchangeably. A polymerization catalyst system is a catalyst system that can polymerize monomers into polymer. An “anionic ligand” is a negatively charged ligand which donates one or more pairs of electrons to a metal ion. A “neutral donor ligand” is a neutrally charged ligand which donates one or more pairs of electrons to a metal ion.

A metallocene catalyst is defined as an organometallic compound with at least one π-bound cyclopentadienyl moiety (or substituted cyclopentadienyl moiety) and more frequently two π-bound cyclopentadienyl moieties or substituted cyclopentadienyl moieties.

For purposes of the present disclosure, in relation to metallocene catalyst compounds, the term “substituted” means that a hydrogen group has been replaced with a hydrocarbyl group, a heteroatom, or a heteroatom containing group. For example, methyl cyclopentadiene (Cp) is a Cp group substituted with a methyl group.

“Catalyst productivity” is a measure of how many grams of polymer (P) are produced using a polymerization catalyst comprising W g of catalyst (cat), over a period of time of T hours; and may be expressed by the following formula: P/(T×W) and expressed in units of gPgcat⁻¹ hr⁻¹. “Conversion” is the amount of monomer that is converted to polymer product, and is reported as mol % and is calculated based on the polymer yield and the amount of monomer fed into the reactor. “Catalyst activity” is a measure of the level of activity of the catalyst and is reported as the mass of product polymer (P) produced per mole (or mmol) of catalyst (cat) used (kgP/molcat or gP/mmolCat), and catalyst activity can also be expressed per unit of time, for example, per hour (hr).

For purposes herein an “olefin,” alternatively referred to as “alkene,” is a linear, branched, or cyclic compound comprising carbon and hydrogen having at least one double bond. For purposes of this specification and the claims appended thereto, when a polymer or copolymer is referred to as comprising an olefin, the olefin present in such polymer or copolymer is the polymerized form of the olefin. For example, when a copolymer is said to have a “propylene” content of 35 wt % to 55 wt %, it is understood that the mer unit in the copolymer is derived from propylene in the polymerization reaction and the derived units are present at 35 wt % to 55 wt %, based upon the weight of the copolymer.

For purposes herein a “polymer” has two or more of the same or different monomer (“mer”) units. A “homopolymer” is a polymer having mer units that are the same. A “copolymer” is a polymer having two or more mer units that are different from each other. A “terpolymer” is a polymer having three mer units that are different from each other. “Different” in reference to mer units indicates that the mer units differ from each other by at least one atom or are different isomerically. Accordingly, copolymer, as used herein, can include terpolymers and the like. An oligomer is typically a polymer having a low molecular weight, such as Mn of less than 25,000 g/mol, or less than 2,500 g/mol, or a low number of mer units, such as 75 mer units or less or 50 mer units or less. An “ethylene polymer” or “ethylene copolymer” is a polymer or copolymer comprising at least 50 mole % ethylene derived units, a “propylene polymer” or “propylene copolymer” is a polymer or copolymer comprising at least 50 mole % propylene derived units, and so on.

As used herein, Mn is number average molecular weight, Mw is weight average molecular weight, and Mz is z average molecular weight, wt % is weight percent, and mol % is mole percent. Molecular weight distribution (MWD), also referred to as polydispersity index (PDI), is defined to be Mw divided by Mn. Furthermore, Mz/Mn indicates viscosity of a polymer. For example, a high Mz/Mn value indicates a low viscosity whereas a low Mz/Mn value indicates high viscosity. Accordingly, a polymer with a larger Mz/Mn ratio would be expected to have a lower viscosity at high shear rates than a polymer with a similar weight average molecular weight but a smaller Mz/Mn ratio.

The term “continuous” means a system that operates without interruption or cessation for a period of time, such as where reactants are continually fed into a reaction zone and products are continually or regularly withdrawn without stopping the reaction in the reaction zone. For example, a continuous process to produce a polymer would be one where the reactants are continually introduced into one or more reactors and polymer product is continually withdrawn.

A “solution polymerization” means a polymerization process in which the polymerization is conducted in a liquid polymerization medium, such as an inert solvent or monomer(s) or their blends. A solution polymerization is typically homogeneous. A homogeneous polymerization is one where the polymer product is dissolved in the polymerization medium. Such systems are typically not turbid as described in Oliveira, J. Vladimir et al. (2000) “High-Pressure Phase Equilibria for Polypropylene-Hydrocarbon Systems,” Ind. Eng. Chem. Res., v 29, pp. 4627-4633.

A bulk polymerization means a polymerization process in which the monomers and/or comonomers being polymerized are used as a solvent or diluent using little or no inert solvent or diluent. A small fraction of inert solvent might be used as a carrier for catalyst and scavenger. A bulk polymerization system contains less than about 25 wt % of inert solvent or diluent, such as less than about 10 wt %, such as less than about 1 wt %, such as 0 wt %.

### Activators

The present disclosure provides ammonium borate activator compounds comprising ammonium groups with long-chain aliphatic hydrocarbyl groups. A borate of the present disclosure is a tetrakis(fluoronaphthyl)borate or tetrakis(fluorophenyl)borate. When an activator of the present disclosure is used with catalyst compound (such as a group 4 metallocene catalyst) in an olefin polymerization, a polymer can be formed having a higher molecular weight and melt temperature than polymers formed using comparative activators. In addition, it has been discovered that activators of the present disclosure are soluble in aliphatic solvent.

In at least one embodiment, an activator is represented by Formula (AI):

[R¹R²R³EH]_{d}⁺[M^{k+}Qₙ]^{d-} (AI)

wherein:
E is nitrogen or phosphorous, preferably nitrogen;
R¹ is an electron deficient aromatic group;
each of R² and R³ is independently C₁-C₄₀ alkyl, C₅-C₂₂-aryl, wherein each of R¹, R², and R³ is independently unsubstituted or substituted with at least one of halide, C₁-C₁₀ alkyl, C₅-C₁₅ aryl, C₆-C₂₅ arylalkyl, and C₆-C₂₅ alkylaryl, wherein R¹, R², and R³ together comprise 15 or more carbon atoms; and
d is 1, 2, or 3; k is 1, 2, or 3; n is 1, 2, 3, 4, 5, or 6; n−k=d (preferably d is 1, 2 or 3; k is 3; n is 4, 5, or 6);
M is an element selected from group 13 of the Periodic Table of the Elements; and
each Q is independently a hydride, bridged or unbridged dialkylamido, halide, alkoxide, aryloxide, hydrocarbyl, substituted hydrocarbyl, halocarbyl, substituted halocarbyl, or halosubstituted-hydrocarbyl radical.

In at least one embodiment, an activator is an ionic ammonium borate represented by Formula (I):

[R¹R²R³EH]⁺[BR⁴R⁵R⁶R⁷]⁻ (I)

wherein:
E is nitrogen or phosphorous;
R¹ is an electron deficient aromatic group (preferably a phenyl group substituted with one to five halogen or haloalkyl groups or a naphthyl group substituted with one to seven halogen or haloalkyl groups);
each of R⁴, R⁵, R⁶, and R⁷ is aryl (such as phenyl or naphthyl), wherein at least one of R⁴, R⁵, R⁶, and R⁷ is substituted with from one or more fluorine atoms; and
each of R² and R³ is independently C₁-C₄₀ alkyl, C₅-C₂₂-aryl, arylalkyl where the alkyl has from 1 to 10 carbon atoms and the aryl has from 6 to 20 carbon atoms, or five-, six- or seven-membered heterocyclyl comprising at least one atom selected from N, P, O and S, wherein
each of R² and R³ is optionally substituted by halogen, —NR′₂, —OR′ or —SiR″₃, wherein R² optionally bonds with R⁵ to independently form a five-, six- or seven-membered ring. R¹, R², and R³ together comprise 15 or more carbon atoms, such as 18 or more carbon atoms, such as 20 or more carbon atoms, such as 22 or more carbon atoms, such as 25 or more carbon atoms, such as 30 or more carbon atoms, such as 35 or more carbon atoms, such as 40 or more carbon atoms, such as 15 to 100 carbon atoms, such as 20 to 75 carbon atoms.

In at least one embodiment, R¹ and R² are independently C₁-C₂₂-alkyl, substituted C₁-C₂₂-alkyl, unsubstituted phenyl, or substituted phenyl. In at least one embodiment, each of R² and R³ is independently selected from methyl, ethyl, n-propyl, n-butyl, n-pentyl, n-hexyl, n-heptyl, n-octyl, n-nonyl, n-decyl, n-undecyl, n-dodecyl, n-tridecyl, n-butadecyl, n-pentadecyl, n-hexadecyl, n-heptadecyl, n-octadecyl, n-nonadecyl, cyclohexylmethyl, and n-icosyl.

In a preferred embodiment of the invention, each of R⁴, R⁵, R⁶, and R⁷ is independently aryl, wherein at least one of R⁴, R⁵, R⁶, and R⁷ is naphthyl substituted with from one to seven fluorine atoms or phenyl substituted with from one to five fluorine atoms. In at least one embodiment, each of R⁴, R⁵, R⁶, and R⁷ is phenyl or naphthyl, wherein at least one of R⁴, R⁵, R⁶, and R⁷ is substituted with from one to seven fluorine atoms. In a preferred embodiment of the invention, each of R⁴, R⁵, R⁶, and R⁷ is independently aryl, wherein at least one of R⁴, R⁵, R⁶, and R⁷ is perfluorinated phenyl or perfluorinated naphthyl, preferably all of R⁴, R⁵, R⁶, and R⁷ are perfluorinated phenyl or perfluorinated naphthyl.

In at least one embodiment, R¹ is an aryl group that is substituted with one, two, three, four, five, or more halogen and/or haloalkyl groups. In at least one embodiment R¹ is a phenyl group that is substituted with one, two, three, four or five halogen and/or haloalkyl groups. In at least one embodiment R¹ is an aryl group, preferably a phenyl group, that is substituted with one, two, three, four or five groups selected from fluoro, chloro, bromo, iodo, and trifluoromethyl. In at least one embodiment R¹ is selected from 4-fluorophenyl, 4-(trifluoromethyl)phenyl, 3-fluorophenyl, 3-(trifluoromethyl)phenyl, and 3-chlorophenyl.

In at least one embodiment, each of R⁴, R⁵, R⁶, and R⁷ is independently a naphthyl comprising one fluorine atom, two fluorine atoms, three fluorine atoms, four fluorine atoms, five fluorine atoms, six fluorine atoms, or seven fluorine atoms.

In at least one embodiment, each of R⁴, R⁵, R⁶, and R⁷ is independently a phenyl comprising one fluorine atom, two fluorine atoms, three fluorine atoms, four fluorine atoms, or five fluorine atoms.

In at least one embodiment, R⁴ is independently naphthyl comprising one fluorine atom, two fluorine atoms, three fluorine atoms, four fluorine atoms, five fluorine atoms, six fluorine atoms, or seven fluorine atoms, and each of R⁵, R⁶, and R⁷ is independently phenyl comprising one fluorine atom, two fluorine atoms, three fluorine atoms, four fluorine atoms, or five fluorine atoms or naphthyl comprising one fluorine atom, two fluorine atoms, three fluorine atoms, four fluorine atoms, five fluorine atoms, six fluorine atoms, or seven fluorine atoms.

In at least one embodiment, M is B or Al, preferably B.

In at least one embodiment, d is 1, 2 or 3; k is 3; and n is 4.

In at least one embodiment, each Q is independently as defined for R⁴.

In at least one embodiment, each Q is a fluorinated hydrocarbyl group having 1 to 30 carbon atoms, more preferably each Q is a fluorinated aryl (such as phenyl or naphthyl) group, and most preferably each Q is a perflourinated aryl (such as phenyl or naphthyl) group. In preferred embodiments of the invention, at least one Q is not substituted phenyl, such as perfluorophenyl, preferably all Q are not substituted phenyl, such as perfluorophenyl.

The terms “cocatalyst” and “activator” are used herein interchangeably and are defined to be any compound which can activate any one of the catalyst compounds of the present disclosure by converting the neutral catalyst compound to a catalytically active catalyst compound cation.

Catalyst systems of the present disclosure may be formed by combining the catalysts with activators in any suitable manner, including by supporting them for use in slurry or gas phase polymerization. The catalyst systems may also be added to or generated in solution polymerization or bulk polymerization (in the monomer, i.e., no solvent).

Both the cation part of formula (I) as well as the anion part thereof, which is an NCA, will be further illustrated below. Any combinations of cations and NCAs disclosed herein are suitable to be used in the processes of the present disclosure and are thus incorporated herein.

### Activators—The Cations

The cation component of the activator of formula (I) above is a protonated Lewis base that can be capable of protonating a moiety, such as an alkyl or aryl, from the transition metal compound. Thus, upon release of a neutral leaving group (e.g. an alkane resulting from the combination of a proton donated from the cationic component of the activator and an alkyl substituent of the transition metal compound) transition metal cation results, which is the catalytically active species.

In at least one embodiment of formula (I), E is nitrogen or phosphorous, and R¹ is an aryl group (such as phenyl or naphthyl) that is substituted with at least one halogen or haloalkyl groups, and each of R² and R³ is independently C₁-C₄₀ alkyl, C₅-C₂₂-aryl, arylalkyl alkyl has from 1 to 10 carbon atoms and aryl has from 6 to 20 carbon atoms, or five-, six- or seven-membered heterocyclyl comprising at least one atom selected from N, P, O and S, wherein each of R² and R³ is optionally substituted by halogen, —NR′₂, —OR′ or —SiR″₃, wherein R² optionally bonds with R⁵ to independently form a five-, six- or seven-membered ring. R¹, R², and R³ together comprise 15 or more carbon atoms, such as 18 or more carbon atoms, such as 20 or more carbon atoms, such as 22 or more carbon atoms, such as 25 or more carbon atoms, such as 30 or more carbon atoms, such as 35 or more carbon atoms, such as 40 or more carbon atoms. In at least one embodiment, R¹ is an aryl group, preferably a phenyl group, that is substituted with one, two, three, four or five groups selected from fluoro, chloro, bromo, iodo, and trifluoromethyl (preferably R¹ is selected from 4-fluorophenyl, 4-(trifluoromethyl)phenyl, 3-fluorophenyl, 3-(trifluoromethyl)phenyl, and 3-chlorophenyl) and R² and R³ are independently substituted or unsubstituted C₁-C₂₂ linear alkyl, or substituted or unsubstituted phenyl. In at least one embodiment, each of R² and R³ is independently selected from methyl, ethyl, n-propyl, n-butyl, n-pentyl, n-hexyl, n-heptyl, n-octyl, n-nonyl, n-decyl, n-undecyl, n-dodecyl, n-tridecyl, n-butadecyl, n-pentadecyl, n-hexadecyl, n-heptadecyl, n-octadecyl, n-nonadecyl, cyclohexylmethyl, and n-icosyl.

Usefully, the compound represented by formula (I) comprises a cation, [R¹R²R³EH]⁺, selected from the group consisting of:
N,N-didodecyl-2,3,4,5,6-pentafluorobenzenaminium,
N,N-didodecyl-3,5-difluorobenzenaminium,
N,N-didodecyl-3,5-bis(trifluoromethyl)benzenaminium,
N,N-bis(cyclohexylmethyl)-2,3,4,5,6-pentafluorobenzenaminium,
N,N-bis(cyclohexylmethyl)-3,5-bis(trifluoromethyl)benzenaminium,
N,N-bis(cyclohexylmethyl)-4-(trifluoromethyl)benzenaminium,
N,N-bis(cyclohexylmethyl)-4-fluorobenzenaminium, and
N,N-didodecyl-4-(trifluoromethyl)anilium.

Preferably, the compound represented by formula (I) comprises a cation, [R¹R²R³EH]⁺, selected from the group consisting of: N,N-bis(cyclohexylmethyl)-4-(trifluoromethyl)benzenaminium, N,N-bis(cyclohexylmethyl)-4-fluorobenzenaminium, and N,N-didodecyl-4-(trifluoromethyl)anilium.

### Activators—The Non-Coordinating Anion (NCA)

A non-coordinating anion (NCA) is an anion that either does not coordinate to the catalyst metal cation or that does coordinate to the metal cation, but only weakly. The term NCA is also defined to include multicomponent NCA-containing activators, such as N,N-dimethylanilinium tetrakis(perfluoronaphthyl)borate, that contain an acidic cationic group and the non-coordinating anion. The term NCA can include neutral Lewis acids, such as tris(perfluoronaphthyl)boron, that can react with a catalyst to form an activated species by abstraction of an anionic group. An NCA coordinates weakly enough that a neutral Lewis base, such as an olefinically or acetylenically unsaturated monomer can displace it from the catalyst center. Any metal or metalloid that can form a compatible, weakly coordinating complex may be used or contained in the non-coordinating anion. Suitable metals can include aluminum, gold, and platinum. Suitable metalloids can include boron, aluminum, phosphorus, and silicon. A stoichiometric activator can be either neutral or ionic. The terms ionic activator, and stoichiometric ionic activator can be used interchangeably. Likewise, the terms neutral stoichiometric activator, and Lewis acid activator can be used interchangeably. The term non-coordinating anion includes neutral stoichiometric activators, ionic stoichiometric activators, ionic activators, and Lewis acid activators.

“Compatible” non-coordinating anions can be those which are not degraded to neutrality when the initially formed complex decomposes. Further, the anion will not transfer an anionic substituent or fragment to the cation so as to cause it to form a neutral transition metal compound and a neutral by-product from the anion. Non-coordinating anions useful in accordance with the present disclosure are those that are compatible, stabilize the transition metal cation in the sense of balancing its ionic charge at +1, and yet retain sufficient lability to permit displacement during polymerization.

The anion component of the activators described herein includes those represented by the formula [M^{k}+Qₙ]⁻ wherein k is 1, 2, or 3; n is 1, 2, 3, 4, 5, or 6 (preferably 1, 2, 3, or 4), (preferably k is 3; n is 4, 5, or 6, preferably when M is B, n is 4); M is an element selected from Group 13 of the Periodic Table of the Elements, preferably boron or aluminum, and Q is independently a hydride, bridged or unbridged dialkylamido, halide, alkoxide, aryloxide, hydrocarbyl, substituted hydrocarbyl, halocarbyl, substituted halocarbyl, and halosubstituted-hydrocarbyl radicals, said Q having up to 20 carbon atoms with the proviso that in not more than 1 occurrence is Q a halide. Preferably, each Q is a fluorinated hydrocarbyl group, optionally having 1 to 20 carbon atoms, more preferably each Q is a fluorinated aryl group, and most preferably each Q is a perfluorinated aryl group. Preferably at least one Q is not substituted phenyl, such as perfluorophenyl, preferably all Q are not substituted phenyl, such as perfluorophenyl.

In at least one embodiment, for the borate moiety ([BR⁴R⁵R⁶R⁷]⁻) of the activator represented by formula (I):

each of R⁴, R⁵, R⁶, and R⁷ is independently aryl- or naphthyl, wherein at least one of R⁴, R⁵, R⁶, and R⁷ is naphthyl substituted with from one to seven fluorine atoms. In at least one embodiment, each of R⁴, R⁵, R⁶, and R⁷ is naphthyl, wherein at least one of R⁴, R⁵, R⁶, and R⁷ is substituted with from one to seven fluorine atoms.

In at least one embodiment, each of R⁴, R⁵, R⁶, and R⁷ is independently naphthyl comprising one fluorine atom, two fluorine atoms, three fluorine atoms, four fluorine atoms, five fluorine atoms, six fluorine atoms, or seven fluorine atoms.

In at least one embodiment, R⁴ is independently naphthyl comprising one fluorine atom, two fluorine atoms, three fluorine atoms, four fluorine atoms, five fluorine atoms, six fluorine atoms, or seven fluorine atoms, and each of R⁵, R⁶, and R⁷ is independently phenyl comprising one fluorine atom, two fluorine atoms, three fluorine atoms, four fluorine atoms, or five fluorine atoms or naphthyl comprising one fluorine atom, two fluorine atoms, three fluorine atoms, four fluorine atoms, five fluorine atoms, six fluorine atoms, or seven fluorine atoms.

In one embodiment, the borate activator comprises tetrakis(heptafluoronaphth-2-yl)borate.

Preferred anions for use in the non-coordinating anion activators described herein include those represented by Formula 7 below:

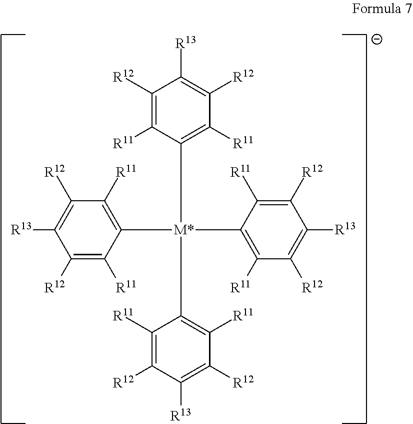

wherein:

M* is a group 13 atom, preferably B or Al, preferably B;

each R¹¹ is, independently, a halide, preferably a fluoride;

each R¹² is, independently, a halide, a C₆ to C₂₀ substituted aromatic hydrocarbyl group or a siloxy group of the formula —O—Si—R^{a}, where R^{a} is a C₁ to C₂₀ hydrocarbyl or hydrocarbylsilyl group, preferably R¹² is a fluoride or a perfluorinated phenyl group;

each R¹³ is a halide, a C₆ to C₂₀ substituted aromatic hydrocarbyl group or a siloxy group of the formula —O—Si—R^{a}, where R^{a} is a C₁ to C₂₀ hydrocarbyl or hydrocarbylsilyl group, preferably R¹³ is a fluoride or a C₆ perfluorinated aromatic hydrocarbyl group;

wherein R¹² and R¹³ can form one or more saturated or unsaturated, substituted or unsubstituted rings, preferably R¹² and R¹³ form a perfluorinated phenyl ring. Preferably the anion has a molecular weight of greater than 700 g/mol, and, preferably, at least three of the substituents on the M* atom each have a molecular volume of greater than 180 cubic A.

“Molecular volume” is used herein as an approximation of spatial steric bulk of an activator molecule in solution. Comparison of substituents with differing molecular volumes allows the substituent with the smaller molecular volume to be considered “less bulky” in comparison to the substituent with the larger molecular volume. Conversely, a substituent with a larger molecular volume may be considered “more bulky” than a substituent with a smaller molecular volume.

Molecular volume may be calculated as reported in Girolami, G. S. (1994) “A Simple “Back of the Envelope” Method for Estimating the Densities and Molecular Volumes of Liquids and Solids,” Journal of Chemical Education, v 71(11), pp. 962-964. Molecular volume (MV), in units of cubic A, is calculated using the formula: MV=8.3V_{S}, where V_{S} is the scaled volume. V_{S} is the sum of the relative volumes of the constituent atoms, and is calculated from the molecular formula of the substituent using Table A below of relative volumes. For fused rings, the V_{S} is decreased by 7.5% per fused ring. The Calculated Total MV of the anion is the sum of the MV per substituent, for example, the MV of perfluorophenyl is 183 Å³, and the Calculated Total MV for tetrakis(perfluorophenyl)borate is four times 183 Å³, or 732 Å³.

TABLE A

| Element | Relative Volume |
|---|---|
| H | 1 |
| 1^{st} short period, Li to F | 2 |
| 2^{nd} short period, Na to Cl | 4 |
| 1^{st} long period, K to Br | 5 |
| 2^{nd} long period, Rb to I | 7.5 |
| 3^{rd} long period, Cs to Bi | 9 |

Exemplary anions useful herein and their respective scaled volumes and molecular volumes are shown in Table 2 below. The dashed bonds indicate bonding to boron.

TABLE 2

| | | Molecular | | MV | |
|---|---|---|---|---|---|
| | | Formula | | Per | Calculated |
| | | of Each | | subst. | Total MV |
| Ion | Structure of Boron Substituents | Substituent | V_{S} | (Å³) | (Å³) |
| tetrakis(perfluorophenyl)borate | 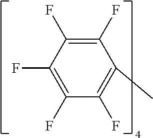 | C₆F₅ | 22 | 183 | 732 |
| tris(perfluorophenyl)- (perfluoronaphthyl)borate | 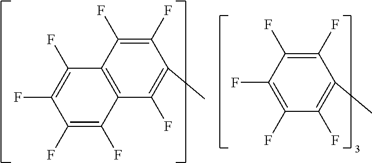 | C₆F₅ C₁₀F₇ | 22 34 | 183 261 | 810 |
| (perfluorophenyl)tris- (perfluoronaphthyl)borate | 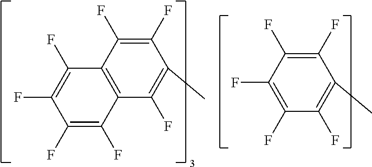 | C₆F₅ C₁₀F₇ | 22 34 | 183 261 | 966 |
| tetrakis(perfluoronaphthyl)borate | 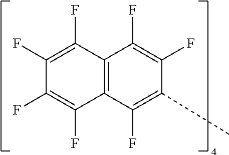 | C₁₀F₇ | 34 | 261 | 1044 |
| tetrakis(perfluorobiphenyl)borate | 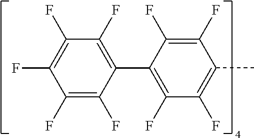 | C₁₂F₉ | 42 | 349 | 1396 |
| [(C₆F₃(C₆F₅)₂)₄B] | 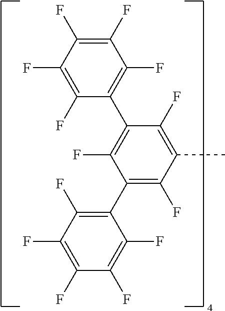 | C₁₈F₁₃ | 62 | 515 | 2060 |

The activators may be added in the form of an ion pair using, for example, [M2HTH]+ [NCA]− in which the Di(hydrogenated tallow)methylamine (“M2HTH”) cation reacts with a basic leaving group on the transition metal complex to form a transition metal complex cation and [NCA]−. Alternatively, the transition metal complex may be reacted with a neutral NCA precursor, such as B(C₁₀F₇)₃, which abstracts an anionic group from the complex to form an activated species. Useful activators include di(hydrogenated tallow)methylamine(perfluoronaphthyl)borate (i.e., [M2HTH]B(C₁₀F₇)₄) and di(octadecyl)tolylamine (perfluoronaphthyl)borate (i.e., [DOdTH]B(C₁₀C₇)₄).

In at least one embodiment, the NCAs purchased under their salt form used for a borate activator compound are: Lithium tetrakis(heptafluoronaphthalen-2-yl)borate etherate (Li—BF28), N,N-Dimethylanilinium tetrakis(heptafluoronaphthalen-2-yl)borate (DMAH-BF28), Sodium tetrakis(heptafluoronaphthalen-2-yl)borate (Na—BF28) and N,N-dimethylanilinium tetrakis(heptafluoronaphthalen-2-yl)borate (DMAH-BF28).

In at least one embodiment, an activator of the present disclosure, when combined with a catalyst (such as a group 4 metallocene) to form an active olefin polymerization catalyst, produces a higher molecular weight polymer (e.g., Mw) than comparative activators that use other borate anions.

In at least one embodiment, the general synthesis of the ammonium borate activators can be performed using a two-step process. In the first step, an amine is dissolved in a solvent (e.g. hexane, cyclohexane, methylcyclohexane, ether, dichloromethane, toluene) and an excess (e.g., 1.2 molar equivalents) of hydrogen chloride is added to form an ammonium chloride salt. This salt is typically isolated by filtration from the reaction medium and dried under reduced pressure. The isolated ammonium chloride is then heated to reflux with about one molar equivalent of an alkali metal borate in a solvent (e.g. cyclohexane, dichloromethane, methylcyclohexane) to form the ammonium borate along with byproduct alkali metal chloride, the latter which can typically be removed by filtration.

In at least one embodiment, an activator of the present disclosure is soluble in an aliphatic solvent at a concentration of about 10 mM or greater, such as about 20 mM or greater, such as about 30 mM or greater, such as about 50 mM or greater, such as about 75 mM or greater, such as about 100 mM or greater, such as about 200 mM or greater, such as about 300 mM or greater. In at least one embodiment, an activator of the present disclosure dissolves in isohexane or methylcyclohexane at 25° C. to form a homogeneous solution of at least 10 mM concentration.

In at least one embodiment, the solubility of the ammonium borate activators of the present disclosure in aliphatic hydrocarbon solvents increases with the number of aliphatic carbons in the ammonium group. In at least one embodiment, a solubility of at least 10 mM is achieved with an activator having an ammonium group of about 21 aliphatic carbon atoms or more, such as about 25 aliphatic carbons atoms or more, such as about 35 carbon atoms or more.

An aliphatic hydrocarbon solvent can be isobutane, butane, pentane, isopentane, hexanes, isohexane, heptane, octane, dodecane, and mixtures thereof; cyclic and alicyclic hydrocarbons, such as cyclohexane, cycloheptane, methylcyclohexane, methylcycloheptane, and mixtures thereof. In at least one embodiment, aromatics are present in the solvent at less than 1 wt %, such as less than 0.5 wt %, such as at 0 wt % based upon the weight of the solvents. The activators of the present disclosure can be dissolved in one or more additional solvents. Additional solvent includes ethereal, halogenated and N,N-dimethylformamide solvents.

In at least one embodiment, an aliphatic solvent is isohexane or methylcyclohexane. In one embodiment, the borate activator is tetrakis(heptafluoronaphth-2-yl)borate.

The typical activator-to-catalyst ratio, e.g., all NCA activators-to-catalyst ratio is about a 1:1 molar ratio. Alternate preferred ranges include from 0.1:1 to 100:1, alternately from 0.5:1 to 200:1, alternately from 1:1 to 500:1 alternately from 1:1 to 1,000:1. A particularly useful range is from 0.5:1 to 10:1, preferably 1:1 to 5:1.

It is also within the scope of the present disclosure that the catalyst compounds can be combined with combinations of alumoxanes and the activators described herein.

### Optional Scavengers or Co-Activators

In addition to these activator compounds, scavengers or co-activators may be used. Aluminum alkyl or organoaluminum compounds which may be utilized as scavengers or co-activators include, for example, trimethylaluminum, triethylaluminum, triisobutylaluminum, tri-n-hexylaluminum, tri-n-octylaluminum, and diethyl zinc.

In at least one embodiment, little or no scavenger is used in the process to produce the ethylene polymer. Scavenger (such as trialkyl aluminum) can be present at zero mol %, alternately the scavenger is present at a molar ratio of scavenger metal to transition metal of less than 100:1, such as less than 50:1, such as less than 15:1, such as less than 10:1.

### Transition Metal Compounds

Any transition metal compound capable of catalyzing a reaction, such as a polymerization reaction, upon activation with an activator as described above is suitable for use in polymerization processes of the present disclosure. Transition metal compounds known as metallocenes are exemplary catalyst compounds according to the present disclosure.

### Catalyst Compounds

In at least one embodiment, the present disclosure provides a catalyst system comprising a catalyst compound having a metal atom. The catalyst compound can be a metallocene catalyst compound. The metal can be a Group 3 through Group 12 metal atom, such as Group 3 through Group 10 metal atoms, or lanthanide Group atoms. The catalyst compound having a Group 3 through Group 12 metal atom can be monodentate or multidentate, such as bidentate, tridentate, or tetradentate, where a heteroatom of the catalyst, such as phosphorous, oxygen, nitrogen, or sulfur is chelated to the metal atom of the catalyst. Non-limiting examples include bis(phenolate)s. In at least one embodiment, the Group 3 through Group 12 metal atom is selected from Group 5, Group 6, Group 8, or Group 10 metal atoms. In at least one embodiment, a Group 3 through Group 10 metal atom is selected from Cr, Sc, Ti, Zr, Hf, V, Nb, Ta, Mn, Re, Fe, Ru, Os, Co, Rh, Ir, and Ni. In at least one embodiment, a metal atom is selected from Groups 4, 5, and 6 metal atoms. In at least one embodiment, a metal atom is a Group 4 metal atom selected from Ti, Zr, or Hf. The oxidation state of the metal atom can range from 0 to +7, for example +1, +2, +3, +4, or +5, for example +2, +3 or +4.

Metallocene catalyst compounds as used herein include metallocenes comprising Group 3 to Group 12 metal complexes, such as, Group 4 to Group 6 metal complexes, for example, Group 4 metal complexes. The metallocene catalyst compound of catalyst systems of the present disclosure may be unbridged metallocene catalyst compounds represented by the formula: Cp^{A}Cp^{B}M′X′ₙ, wherein each Cp^{A} and Cp^{B} is independently selected from cyclopentadienyl ligands and ligands isolobal to cyclopentadienyl, one or both Cp^{A} and Cp^{B} may contain heteroatoms, and one or both Cp^{A} and Cp^{B} may be substituted by one or more R″ groups. M′ is selected from Groups 3 through 12 atoms and lanthanide Group atoms. X′ is an anionic leaving group. n is 0 or an integer from 1 to 4. R″ is selected from alkyl, lower alkyl, substituted alkyl, heteroalkyl, alkenyl, lower alkenyl, substituted alkenyl, heteroalkenyl, alkynyl, lower alkynyl, substituted alkynyl, heteroalkynyl, alkoxy, lower alkoxy, aryloxy, alkylthio, lower alkylthio, arylthio, aryl, substituted aryl, heteroaryl, aralkyl, aralkylene, alkaryl, alkarylene, haloalkyl, haloalkenyl, haloalkynyl, heteroalkyl, heterocycle, heteroaryl, a heteroatom-containing group, hydrocarbyl, lower hydrocarbyl, substituted hydrocarbyl, heterohydrocarbyl, silyl, boryl, phosphino, phosphine, amino, amine, ether, and thioether.

In at least one embodiment, each Cp^{A} and Cp^{B} is independently selected from cyclopentadienyl, indenyl, fluorenyl, cyclopentaphenanthreneyl, benzindenyl, fluorenyl, octahydrofluorenyl, cyclooctatetraenyl, cyclopentacyclododecene, phenanthrindenyl, 3,4-benzofluorenyl, 9-phenylfluorenyl, 8-H-cyclopent[a]acenaphthylenyl, 7-H-dibenzofluorenyl, indeno[1,2-9]anthrene, thiophenoindenyl, thiophenofluorenyl, and hydrogenated versions thereof.

The metallocene catalyst compound may be a bridged metallocene catalyst compound represented by the formula: Cp^{A}(A)Cp^{B}M′X′ₙ, wherein each Cp^{A} and Cp^{B} is independently selected from cyclopentadienyl ligands and ligands isolobal to cyclopentadienyl. One or both Cp^{A} and Cp^{B} may contain heteroatoms, and one or both Cp^{A} and Cp^{B} may be substituted by one or more R″ groups. M′ is selected from Groups 3 through 12 atoms and lanthanide Group atoms. X′ is an anionic leaving group. n is 0 or an integer from 1 to 4. (A) is selected from divalent alkyl, divalent lower alkyl, divalent substituted alkyl, divalent heteroalkyl, divalent alkenyl, divalent lower alkenyl, divalent substituted alkenyl, divalent heteroalkenyl, divalent alkynyl, divalent lower alkynyl, divalent substituted alkynyl, divalent heteroalkynyl, divalent alkoxy, divalent lower alkoxy, divalent aryloxy, divalent alkylthio, divalent lower alkylthio, divalent arylthio, divalent aryl, divalent substituted aryl, divalent heteroaryl, divalent aralkyl, divalent aralkylene, divalent alkaryl, divalent alkarylene, divalent haloalkyl, divalent haloalkenyl, divalent haloalkynyl, divalent heteroalkyl, divalent heterocycle, divalent heteroaryl, a divalent heteroatom-containing group, divalent hydrocarbyl, divalent lower hydrocarbyl, divalent substituted hydrocarbyl, divalent heterohydrocarbyl, divalent silyl, divalent boryl, divalent phosphino, divalent phosphine, divalent amino, divalent amine, divalent ether, divalent thioether. R″ is selected from alkyl, lower alkyl, substituted alkyl, heteroalkyl, alkenyl, lower alkenyl, substituted alkenyl, heteroalkenyl, alkynyl, lower alkynyl, substituted alkynyl, heteroalkynyl, alkoxy, lower alkoxy, aryloxy, alkylthio, lower alkylthio, arylthio, aryl, substituted aryl, heteroaryl, aralkyl, aralkylene, alkaryl, alkarylene, haloalkyl, haloalkenyl, haloalkynyl, heteroalkyl, heterocycle, heteroaryl, a heteroatom-containing group, hydrocarbyl, lower hydrocarbyl, substituted hydrocarbyl, heterohydrocarbyl, silyl, boryl, phosphino, phosphine, amino, amine, germanium, ether, and thioether.

In at least one embodiment, each of Cp^{A} and Cp^{B} is independently selected from cyclopentadienyl, n-propylcyclopentadienyl, indenyl, pentamethylcyclopentadienyl, tetramethylcyclopentadienyl, and n-butylcyclopentadienyl.

(A) may be O, S, NR′, or SiR′₂, where each R′ is independently hydrogen or C₁-C₂₀ hydrocarbyl.

In another embodiment, the metallocene catalyst compound is represented by the formula:

T_{y}CpₘMGₙX_{q}

where Cp is independently a substituted or unsubstituted cyclopentadienyl ligand or substituted or unsubstituted ligand isolobal to cyclopentadienyl. M is a Group 4 transition metal. G is a heteroatom group represented by the formula JR*z where J is N, P, O or S, and R* is a linear, branched, or cyclic C₁-C₂₀ hydrocarbyl. z is 1 or 2. T is a bridging group. y is 0 or 1. X is a leaving group. m=1, n=1, 2 or 3, q=0, 1, 2 or 3, and the sum of m+n+q is equal to the oxidation state of the transition metal.

In at least one embodiment, J is N, and R* is methyl, ethyl, propyl, butyl, pentyl, hexyl, heptyl, octyl, nonyl, cyclooctyl, cyclododecyl, decyl, undecyl, dodecyl, adamantyl or an isomer thereof.

In at least one embodiment, the catalyst compound is represented by formula (II) or formula (III):

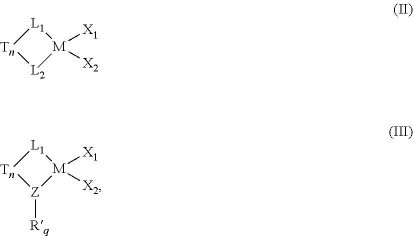

wherein in each of formula (II) and formula (III):
M is the metal center, and is a Group 4 metal, such as titanium, zirconium or hafnium, such as zirconium or hafnium when L₁ and L₂ are present and titanium when Z is present;
n is 0 or 1:
T is an optional bridging group which, if present, is selected from dialkylsilyl, diarylsilyl, dialkylmethyl, ethylenyl (—CH₂—CH₂—) or hydrocarbylethylenyl wherein one, two, three or four of the hydrogen atoms in ethylenyl are substituted by hydrocarbyl, where hydrocarbyl can be independently C₁ to C₁₆ alkyl or phenyl, tolyl, xyly and the like, and when T is present, the catalyst represented can be in a racemic or a meso form,
L₁ and L₂ are independently cyclopentadienyl, indenyl, tetrahydroindenyl or fluorenyl, optionally substituted, that are each bonded to M, or L₁ and L₂ are independently cyclopentadienyl, indenyl, tetrahydroindenyl or fluorenyl, which are optionally substituted, in which any two adjacent substituents on L₁ and L₂ are optionally joined to form a substituted or unsubstituted, saturated, partially unsaturated, or aromatic cyclic or polycyclic substituent;
Z is nitrogen, sulfur, oxygen or phosphorus;

### Q is 1 or 2

R′ is a cyclic, linear or branched C₁ to C₄₀ alkyl or substituted alkyl group (such as Z—R′ form a cyclododecylamido group); X₁ and X₂ are, independently, hydrogen, halogen, hydride radicals, hydrocarbyl radicals, substituted hydrocarbyl radicals, halocarbyl radicals, substituted halocarbyl radicals, silylcarbyl radicals, substituted silylcarbyl radicals, germylcarbyl radicals, or substituted germylcarbyl radicals; or X₁ and X₂ are joined and bound to the metal atom to form a metallacycle ring containing from about 3 to about 20 carbon atoms; or both together can be an olefin, diolefin or aryne ligand.

In at least one embodiment, the catalyst compound has a symmetry that is C₂ symmetrical.

In at least one embodiment, the catalyst compound may be selected from:
bis(1-methyl, 3-n-butyl cyclopentadienyl) M(R)₂;
dimethylsilyl bis(indenyl) M(R)₂;
bis(indenyl) M(R)₂;
dimethylsilyl bis(tetrahydroindenyl) M(R)₂;
bis(n-propylcyclopentadienyl) M(R)₂;
dimethylsilyl (tetramethylcyclopentadienyl)(cyclododecylamido) M(R)₂;
dimethylsilyl (tetramethylcyclopentadienyl)(cyclododecylamido) M(R)₂;
dimethylsilyl (tetramethylcyclopentadienyl)(t-butylamido) M(R)₂;
dimethylsilyl (tetramethylcyclopentadienyl)(t-butylamido) M(R)₂;
μ-(CH₃)₂Si(cyclopentadienyl)(1-adamantylamido) M(R)₂;
μ-(CH₃)₂Si(3-tertbutylcyclopentadienyl)(1-adamantylamido) M(R)₂;
μ-(CH₃)₂ (tetramethylcyclopentadienyl)(1-adamantylamido) M(R)₂;
μ-(CH₃)₂Si(tetramethylcyclopentadienyl)(1-adamantylamido) M(R)₂;
μ-(CH₃)₂C(tetramethylcyclopentadienyl)(1-adamantylamido) M(R)₂;
μ-(CH₃)₂Si(tetramethylcyclopentadienyl)(1-tertbutylamido) M(R)₂;
μ-(CH₃)₂Si(fluorenyl)(1-tertbutylamido) M(R)₂;
μ-(CH₃)₂Si(tetramethylcyclopentadienyl)(1-cyclododecylamido) M(R)₂;
μ-(C₆H₅)₂C(tetramethylcyclopentadienyl)(1-cyclododecylamido) M(R)₂;
μ-(CH₃)₂Si(η⁵-2,6,6-trimethyl-1,5,6,7-tetrahydro-s-indacen-1-yl)(tertbutylamido) M(R)₂;
   where M is selected from Ti, Zr, and Hf; and R is selected from halogen or C₁ to C₅ alkyl.

In at least one embodiment, the catalyst is rac-dimethylsilyl-bis(indenyl)hafnium dimethyl.

### Non-Metallocene Catalyst Compounds

Transition metal complexes for polymerization processes can include any olefin polymerization catalyst. Suitable catalyst components may include “non-metallocene complexes” that are defined to be transition metal complexes that do not feature a cyclopentadienyl anion or substituted cyclopentadienyl anion donors (e.g., cyclopentadienyl, fluorenyl, indenyl, methylcyclopentadienyl). Examples of families of non-metallocene complexes that may be suitable can include late transition metal pyridylbisimines (e.g., U.S. Pat. No. 7,087,686), group 4 pyridyldiamidos (e.g., U.S. Pat. No. 7,973,116), quinolinyldiamidos (e.g., US Pub. No. 2018/0002352 A1), pyridylamidos (e.g., U.S. Pat. No. 7,087,690), phenoxyimines (e.g., Makio, H. et al. (2009) “Development and Application of FI Catalysts for Olefin Polymerization: Unique Catalysis and Distinctive Polymer Formation,” Accounts of Chemical Research, v. 42(10), pp. 1532-1544), and bridged bi-aromatic complexes (e.g., U.S. Pat. No. 7,091,292), the disclosures of which are incorporated herein by reference.

Catalyst complexes that are suitable for use in combination with the activators described herein include: pyridyldiamido complexes; quinolinyldiamido complexes; phenoxyimine complexes; bisphenolate complexes; cyclopentadienyl-amidinate complexes; and iron pyridyl bis(imine) complexes or any combination thereof, including any combination with metallocene complexes

The term “pyridyldiamido complex” or “pyridyldiamide complex” or “pyridyldiamido catalyst” or “pyridyldiamide catalyst” refers to a class of coordination complexes described in U.S. Pat. No. 7,973,116 B2, US 2012/0071616A1, US 2011/0224391A1, US 2011/0301310 A1, US 2015/0141601 A1, U.S. Pat. Nos. 6,900,321 and 8,592,615 that feature a dianionic tridentate ligand that is coordinated to a metal center through one neutral Lewis basic donor atom (e.g., a pyridine group) and a pair of anionic amido or phosphido (i.e., deprotonated amine or phosphine) donors. In these complexes the pyridyldiamido ligand is coordinated to the metal with the formation of one five membered chelate ring and one seven membered chelate ring. It is possible for additional atoms of the pyridyldiamido ligand to be coordinated to the metal without affecting the catalyst function upon activation; an example of this could be a cyclometalated substituted aryl group that forms an additional bond to the metal center.

The term “quinolinyldiamido complex” or “quinolinyldiamido catalyst” or “quinolinyldiamide complex” or “quinolinyldiamide catalyst” refers to a related class of pyridyldiamido complex/catalyst described in US 2018/0002352 where a quinolinyl moiety is present instead of a pyridyl moiety.

The term “phenoxyimine complex” or “phenoxyimine catalyst” refers to a class of coordination complexes described in EP 0874005 that feature a monoanionic bidentate ligand that is coordinated to a metal center through one neutral Lewis basic donor atom (e.g., an imine moiety) and an anionic aryloxy (i.e., deprotonated phenoxy) donor. Typically two of these bidentate phenoxyimine ligands are coordinated to a group 4 metal to form a complex that is useful as a catalyst component.

The term “bisphenolate complex” or “bisphenolate catalyst” refers to a class of coordination complexes described in U.S. Pat. No. 6,841,502, WO2017/004462, and WO2006/020624 that feature a dianionic tetradentate ligand that is coordinated to a metal center through two neutral Lewis basic donor atoms (e.g., oxygen bridge moieties) and two anionic aryloxy (i.e., deprotonated phenoxy) donors.

The term “cyclopentadienyl-amidinate complex” or “cyclopentadienyl-amidinate catalyst” refers to a class of coordination complexes described in U.S. Pat. No. 8,188,200 that typically feature a group 4 metal bound to a cyclopentadienyl anion, a bidentate amidinate anion, and a couple of other anionic groups.

The term “iron pyridyl bis(imine) complex” refers to a class of iron coordination complexes described in U.S. Pat. No. 7,087,686 that typically feature an iron metal center coordinated to a neutral, tridentate pyridyl bis(imine) ligand and two other anionic ligands.

Non-metallocene complexes can include iron complexes of tridentate pyridylbisimine ligands, zirconium and hafnium complexes of pyridylamido ligands, zirconium and hafnium complexes of tridentate pyridyldiamido ligands, zirconium and hafnium complexes of tridentate quinolinyldiamido ligands, zirconium and hafnium complexes of bidentate phenoxyimine ligands, and zirconium and hafnium complexes of bridged bi-aromatic ligands.

Suitable non-metallocene complexes can include zirconium and hafnium non-metallocene complexes. In at least one embodiment, non-metallocene complexes for the present disclosure include group 4 non-metallocene complexes including two anionic donor atoms and one or two neutral donor atoms. Suitable non-metallocene complexes for the present disclosure include group 4 non-metallocene complexes including an anionic amido donor. Suitable non-metallocene complexes for the present disclosure include group 4 non-metallocene complexes including an anionic aryloxide donor atom. Suitable non-metallocene complexes for the present disclosure include group 4 non-metallocene complexes including two anionic aryloxide donor atoms and two additional neutral donor atoms.

A catalyst compounds can be a quinolinyldiamido (QDA) transition metal complex represented by Formula (BI), such as by Formula (BII), such as by Formula (BIII):

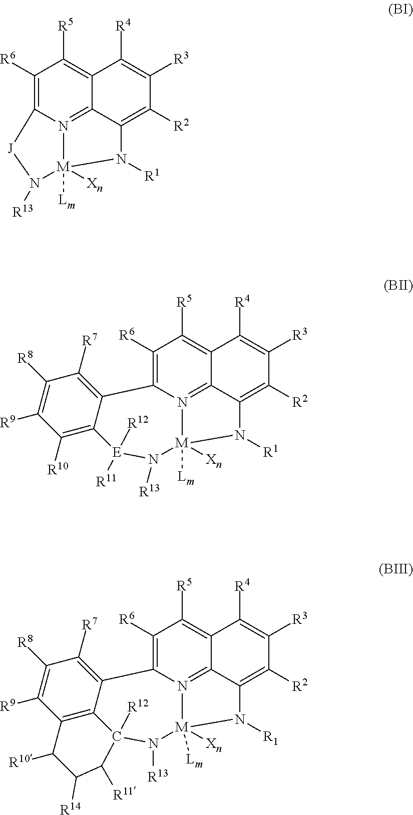

wherein:
M is a group 3, 4, 5, 6, 7, 8, 9, 10, 11, or 12 metal, such as a group 4 metal;
J is group including a three-atom-length bridge between the quinoline and the amido nitrogen, such as a group containing up to 50 non-hydrogen atoms;
E is carbon, silicon, or germanium;
X is an anionic leaving group, (such as a hydrocarbyl group or a halogen);
L is a neutral Lewis base;
R¹ and R¹³ are independently selected from the group including of hydrocarbyls, substituted hydrocarbyls, and silyl groups;
R², R³, R⁴, R⁵, R⁶, R⁷, R⁸, R⁹, R¹⁰, R^{10′}, R¹¹, R^{11′}, R¹², and R¹⁴ are independently hydrogen, hydrocarbyl, alkoxy, silyl, amino, aryloxy, substituted hydrocarbyl, halogen, or phosphino;
n is 1 or 2;
m is 0, 1, or 2, where
n+m is not greater than 4; and
any two R groups (e.g., R¹ & R², R² & R³, R¹⁰ and R¹¹, etc.) may be joined to form a substituted hydrocarbyl, unsubstituted hydrocarbyl, substituted heterocyclic, or unsubstituted heterocyclic, saturated or unsaturated ring, where the ring has 5, 6, 7, or 8 ring atoms and where substitutions on the ring can join to form additional rings;
any two X groups may be joined together to form a dianionic group;
any two L groups may be joined together to form a bidentate Lewis base; and
any X group may be joined to an L group to form a monoanionic bidentate group.

In at least one embodiment, M is a group 4 metal, such as zirconium or hafnium, such as M is hafnium.

Representative non-metallocene transition metal compounds usable for forming poly(alpha-olefin)s of the present disclosure also include tetrabenzyl zirconium, tetra bis(trimethylsilymethyl) zirconium, oxotris(trimethlsilylmethyl) vanadium, tetrabenzyl hafnium, tetrabenzyl titanium, bis(hexamethyl disilazido)dimethyl titanium, tris(trimethyl silyl methyl) niobium dichloride, and tris(trimethylsilylmethyl) tantalum dichloride.

In at least one embodiment, J is an aromatic substituted or unsubstituted hydrocarbyl having from 3 to 30 non-hydrogen atoms, such as J is represented by the formula:

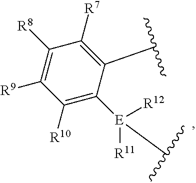

such as J is

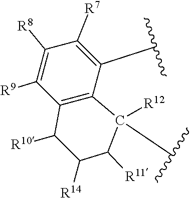

where R⁷, R⁸, R⁹, R¹⁰, R^{10′}, R¹¹, R^{11′}, R¹², R¹⁴ and E are as defined above, and any two R groups (e.g., R⁷ & R⁸, R⁸ & R⁹, R⁹ & R¹⁰, R¹⁰ & R¹¹, etc.) may be joined to form a substituted or unsubstituted hydrocarbyl or heterocyclic ring, where the ring has 5, 6, 7, or 8 ring atoms (such as 5 or 6 atoms), and said ring may be saturated or unsaturated (such as partially unsaturated or aromatic), such as J is an arylalkyl (such as arylmethyl, etc.) or dihydro-1H-indenyl, or tetrahydronaphthalenyl group.

In at least one embodiment, J is selected from the following structures:

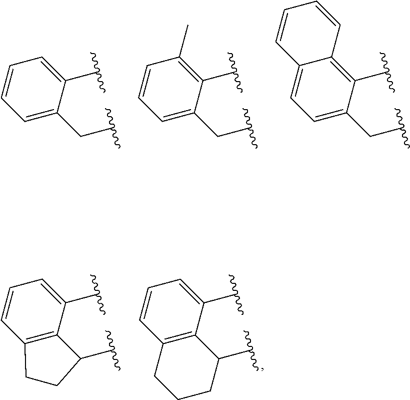

where [symbol] indicates connection to the complex.

In at least one embodiment, E is carbon.

X may be an alkyl (such as alkyl groups having 1 to 10 carbons, such as methyl, ethyl, propyl, butyl, pentyl, hexyl, heptyl, octyl, nonyl, decyl, and isomers thereof), aryl, hydride, alkylsilane, fluoride, chloride, bromide, iodide, triflate, carboxylate, amido (such as NMe₂), or alkylsulfonate.

In at least one embodiment, L is an ether, amine or thioether.

In at least one embodiment, R⁷ and R⁸ are joined to form a six-membered aromatic ring with the joined R⁷/R⁸ group being —CH═CHCH═CH—.

R¹⁰ and R¹¹ may be joined to form a five-membered ring with the joined R¹⁰R¹¹ group being —CH₂CH₂—.

In at least one embodiment, R¹⁰ and R¹¹ are joined to form a six-membered ring with the joined R¹⁰R¹¹ group being —CH₂CH₂CH₂—.

R¹ and R¹³ may be independently selected from phenyl groups that are variously substituted with between zero to five substituents that include F, Cl, Br, I, CF₃, NO₂, alkoxy, dialkylamino, aryl, and alkyl groups having 1 to 10 carbons, such as methyl, ethyl, propyl, butyl, pentyl, hexyl, heptyl, octyl, nonyl, decyl, and isomers thereof.

In at least one embodiment, the QDA transition metal complex represented by the Formula (II) above where:

M is a group 4 metal (such hafnium);
E is selected from carbon, silicon, or germanium (such as carbon);
X is an alkyl, aryl, hydride, alkylsilane, fluoride, chloride, bromide, iodide, triflate, carboxylate, amido, alkoxo, or alkylsulfonate;
L is an ether, amine, or thioether;
R¹ and R¹³ are independently selected from the group consisting of hydrocarbyls, substituted hydrocarbyls, and silyl groups (such as aryl);
R², R³, R⁴, R⁵, R⁶, R⁷, R⁸, R⁹, R¹⁰, R¹¹, and R¹² are independently hydrogen, hydrocarbyl, alkoxy, silyl, amino, aryloxy, substituted hydrocarbyls, halogen, and phosphino;
n is 1 or 2;
m is 0, 1, or 2;
n+m is from 1 to 4;
two X groups may be joined together to form a dianionic group;
two L groups may be joined together to form a bidentate Lewis base;
an X group may be joined to an L group to form a monoanionic bidentate group;
R⁷ and R⁸ may be joined to form a ring (such as an aromatic ring, a six-membered aromatic ring with the joined R⁷R⁸ group being —CH═CHCH═CH—); and
R¹⁰ and R¹¹ may be joined to form a ring (such as a five-membered ring with the joined R¹⁰R¹¹ group being —CH₂CH₂—, a six-membered ring with the joined R¹⁰R¹¹ group being —CH₂CH₂CH₂—).

In at least one embodiment of Formula (BI), (BII), and (BIII), R⁴, R⁵, and R⁶ are independently selected from the group including hydrogen, hydrocarbyls, substituted hydrocarbyls, alkoxy, aryloxy, halogen, amino, and silyl, and wherein adjacent R groups (R⁴ and R⁵ and/or R⁵ and R⁶) are joined to form a substituted hydrocarbyl, unsubstituted hydrocarbyl, unsubstituted heterocyclic ring or substituted heterocyclic ring, where the ring has 5, 6, 7, or 8 ring atoms and where substitutions on the ring can join to form additional rings.

In at least one embodiment of Formula (BI), (BII), and (BIII), R⁷, R⁸, R⁹, and R¹⁰ are independently selected from the group including hydrogen, hydrocarbyls, substituted hydrocarbyls, alkoxy, halogen, amino, and silyl, and wherein adjacent R groups (R⁷ and R⁸ and/or R⁹ and R¹⁰) may be joined to form a saturated, substituted hydrocarbyl, unsubstituted hydrocarbyl, unsubstituted heterocyclic ring or substituted heterocyclic ring, where the ring has 5, 6, 7, or 8 ring carbon atoms and where substitutions on the ring can join to form additional rings.

In at least one embodiment of Formula (BI), (BII), and (BIII), R² and R³ are each, independently, selected from the group including hydrogen, hydrocarbyls, and substituted hydrocarbyls, alkoxy, silyl, amino, aryloxy, halogen, and phosphino, R² and R³ may be joined to form a saturated, substituted or unsubstituted hydrocarbyl ring, where the ring has 4, 5, 6, or 7 ring carbon atoms and where substitutions on the ring can join to form additional rings, or R² and R³ may be joined to form a saturated heterocyclic ring, or a saturated substituted heterocyclic ring where substitutions on the ring can join to form additional rings.

In at least one embodiment of Formula (BI), (BII), and (BIII), R¹¹ and R¹² are each, independently, selected from the group including hydrogen, hydrocarbyls, and substituted hydrocarbyls, alkoxy, silyl, amino, aryloxy, halogen, and phosphino, R¹¹ and R¹² may be joined to form a saturated, substituted or unsubstituted hydrocarbyl ring, where the ring has 4, 5, 6, or 7 ring carbon atoms and where substitutions on the ring can join to form additional rings, or R¹¹ and R¹² may be joined to form a saturated heterocyclic ring, or a saturated substituted heterocyclic ring where substitutions on the ring can join to form additional rings, or R¹¹ and R¹⁰ may be joined to form a saturated heterocyclic ring, or a saturated substituted heterocyclic ring where substitutions on the ring can join to form additional rings.

In at least one embodiment of Formula (BI), (BII), and (BIII), R¹ and R¹³ are independently selected from phenyl groups that are variously substituted with between zero to five substituents that include F, Cl, Br, I, CF₃, NO₂, alkoxy, dialkylamino, aryl, and alkyl groups having 1 to 10 carbons, such as methyl, ethyl, propyl, butyl, pentyl, hexyl, heptyl, octyl, nonyl, decyl, and isomers thereof.

In at least one embodiment of Formula (BII), suitable R¹²-E-R¹¹ groups include CH₂, CMe₂, SiMe₂, SiEt₂, SiPr₂, SiBu₂, SiPh₂, Si(aryl)₂, Si(alkyl)₂, CH(aryl), CH(Ph), CH(alkyl), and CH(2-isopropylphenyl), where alkyl is a C₁ to C₄₀ alkyl group (such as C₁ to C₂₀ alkyl, such as one or more of methyl, ethyl, propyl, butyl, pentyl, hexyl, heptyl, octyl, nonyl, decyl, undecyl, dodecyl, and isomers thereof), aryl is a C₅ to C₄₀ aryl group (such as a C₆ to C₂₀ aryl group, such as phenyl or substituted phenyl, such as phenyl, 2-isopropylphenyl, or 2-tertbutylphenyl).

In at least one embodiment of Formula (BIII), R¹¹, R¹², R⁹, R¹⁴, and R¹⁰ are independently selected from the group consisting of hydrogen, hydrocarbyls, substituted hydrocarbyls, alkoxy, halogen, amino, and silyl, and wherein adjacent R groups (R¹⁰ and R¹⁴, and/or R¹¹ and R¹⁴, and/or R⁹ and R¹⁰) may be joined to form a saturated, substituted hydrocarbyl, unsubstituted hydrocarbyl, unsubstituted heterocyclic ring or substituted heterocyclic ring, where the ring has 5, 6, 7, or 8 ring carbon atoms and where substitutions on the ring can join to form additional rings.

The R groups above (i.e., any of R² to R¹⁴) and other R groups mentioned hereafter may contain from 1 to 30, such as 2 to 20 carbon atoms, such as from 6 to 20 carbon atoms. The R groups above (i.e., any of R² to R¹⁴) and other R groups mentioned hereafter, may be independently selected from the group including hydrogen, methyl, ethyl, phenyl, isopropyl, isobutyl, trimethylsilyl, and —CH₂—Si(Me)₃.

In at least one embodiment, the quinolinyldiamide complex is linked to one or more additional transition metal complex, such as a quinolinyldiamide complex or another suitable non-metallocene, through an R group in such a fashion as to make a bimetallic, trimetallic, or multimetallic complex that may be used as a catalyst component for olefin polymerization. The linker R-group in such a complex may contain 1 to 30 carbon atoms.

In at least one embodiment, E is carbon and R¹¹ and R¹² are independently selected from phenyl groups that are substituted with 0, 1, 2, 3, 4, or 5 substituents selected from the group consisting of F, Cl, Br, I, CF₃, NO₂, alkoxy, dialkylamino, hydrocarbyl, and substituted hydrocarbyl groups with from one to ten carbons.

In at least one embodiment of Formula (BII) or (BIII), R¹¹ and R¹² are independently selected from hydrogen, methyl, ethyl, phenyl, isopropyl, isobutyl, —CH₂—Si(Me)₃, and trimethylsilyl.

In at least one embodiment of Formula (BII), and (BIII), R⁷, R⁸, R⁹, and R¹⁰ are independently selected from hydrogen, methyl, ethyl, propyl, isopropyl, phenyl, cyclohexyl, fluoro, chloro, methoxy, ethoxy, phenoxy, —CH₂—Si(Me)₃, and trimethylsilyl.

In at least one embodiment of Formula (BI), (BII), and (BIII), R², R³, R⁴, R⁵, and R⁶ are independently selected from the group consisting of hydrogen, hydrocarbyls, alkoxy, silyl, amino, substituted hydrocarbyls, and halogen.

In at least one embodiment of Formula (BIII), R¹⁰, R¹¹ and R¹⁴ are independently selected from hydrogen, methyl, ethyl, phenyl, isopropyl, isobutyl, —CH₂—Si(Me)₃, and trimethylsilyl.

In at least one embodiment of Formula (BI), (BII), and (BIII), each L is independently selected from Et₂O, MeOtBu, Et₃N, PhNMe₂, MePh₂N, tetrahydrofuran, and dimethylsulfide.

In at least one embodiment of Formula (BI), (BII), and (BIII), each X is independently selected from methyl, benzyl, trimethylsilyl, neopentyl, ethyl, propyl, butyl, phenyl, hydrido, chloro, fluoro, bromo, iodo, dimethylamido, diethylamido, dipropylamido, and diisopropylamido.

In at least one embodiment of Formula (BI), (BII), and (BIII), R¹ is 2,6-diisopropylphenyl, 2,4,6-triisopropylphenyl, 2,6-diisopropyl-4-methylphenyl, 2,6-diethylphenyl, 2-ethyl-6-isopropylphenyl, 2,6-bis(3-pentyl)phenyl, 2,6-dicyclopentylphenyl, or 2,6-dicyclohexylphenyl.

In at least one embodiment of Formula (BI), (BII), and (BIII), R¹³ is phenyl, 2-methylphenyl, 2-ethylphenyl, 2-propylphenyl, 2,6-dimethylphenyl, 2-isopropylphenyl, 4-methylphenyl, 3,5-dimethylphenyl, 3,5-di-tert-butylphenyl, 4-fluorophenyl, 3-methylphenyl, 4-dimethylaminophenyl, or 2-phenylphenyl.

In at least one embodiment of Formula (BII), J is dihydro-1H-indenyl and R¹ is 2,6-dialkylphenyl or 2,4,6-trialkylphenyl.

In at least one embodiment of Formula (BI), (BII), and (BIII), R¹ is 2,6-diisopropylphenyl and R¹³ is a hydrocarbyl group containing 1, 2, 3, 4, 5, 6, or 7 carbon atoms.

An exemplary catalyst used for polymerizations of the present disclosure is (QDA-1)HfMe₂, as described in US 2018/0002352 A1.

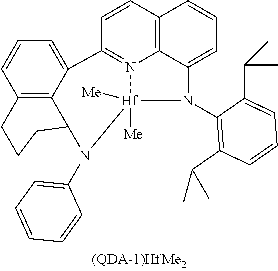

In at least one embodiment, the catalyst compound is a bis(phenolate) catalyst compound represented by Formula (CI):

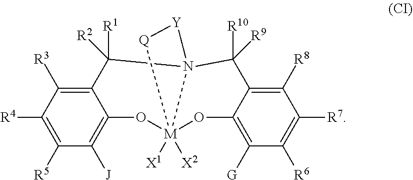

M is a Group 4 metal, such as Hf or Zr. X¹ and X² are independently a univalent C₁-C₂₀ hydrocarbyl, C₁-C₂₀ substituted hydrocarbyl, a heteroatom or a heteroatom-containing group, or X¹ and X² join together to form a C₄-C₆₂ cyclic or polycyclic ring structure. R¹, R², R³, R⁴, R⁵, R⁶, R⁷, R⁸, R⁹, and R¹⁰ is independently hydrogen, C₁-C₄₀ hydrocarbyl, C₁-C₄₀ substituted hydrocarbyl, a heteroatom or a heteroatom-containing group, or two or more of R¹, R², R³, R⁴, R⁵, R⁶, R⁷, R⁸, R⁹, and R¹⁰ are joined together to form a C₄-C₆₂ cyclic or polycyclic ring structure, or a combination thereof, Q is a neutral donor group; J is heterocycle, a substituted or unsubstituted C₇-C₆₀ fused polycyclic group, where at least one ring is aromatic and where at least one ring, which may or may not be aromatic, has at least five ring atoms' G is as defined for J or may be hydrogen, C₂-C₆₀ hydrocarbyl, C₁-C₆₀ substituted hydrocarbyl, or may independently form a C₄-C₆₀ cyclic or polycyclic ring structure with R⁶, R⁷ or R⁸ or a combination thereof, Y is divalent C₁-C₂₀ hydrocarbyl or divalent C₁-C₂₀ substituted hydrocarbyl or (-Q-Y—) together form a heterocycle; and heterocycle may be aromatic and/or may have multiple fused rings.

In at least one embodiment, the catalyst compound represented by Formula (CI) is represented by Formula (CII) or Formula (CIII):

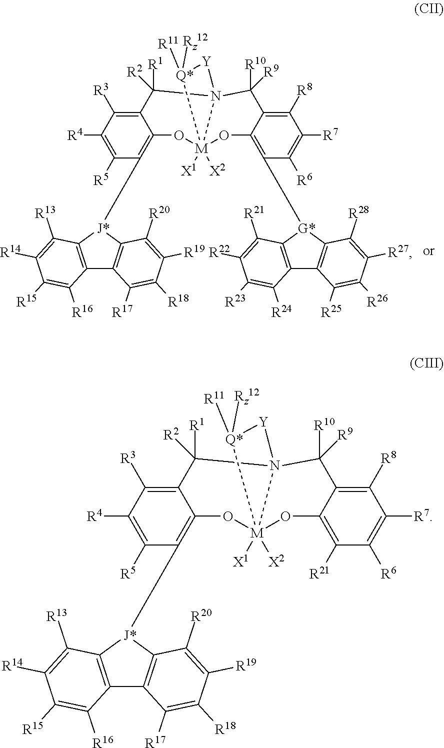

M is Hf, Zr, or Ti. X¹, X², R¹, R², R³, R⁴, R⁵, R⁶, R⁷, R⁸, R⁹, R¹⁰, and Y are as defined for Formula (CI). R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, and R²⁸ is independently a hydrogen, C₁-C₄₀ hydrocarbyl, C₁-C₄₀ substituted hydrocarbyl, a functional group comprising elements from Groups 13 to 17, or two or more of R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, and R²⁸ may independently join together to form a C₄-C₆₂ cyclic or polycyclic ring structure, or a combination thereof; R¹¹ and R¹² may join together to form a five- to eight-membered heterocycle; Q* is a group 15 or 16 atom; z is 0 or 1; J* is CR″ or N, and G* is CR″ or N, where R″ is C₁-C₂₀ hydrocarbyl or carbonyl-containing C₁-C₂₀ hydrocarbyl; and z=0 if Q* is a group 16 atom, and z=1 if Q* is a group 15 atom.

In at least one embodiment the catalyst is an iron complex represented by formula (IV):

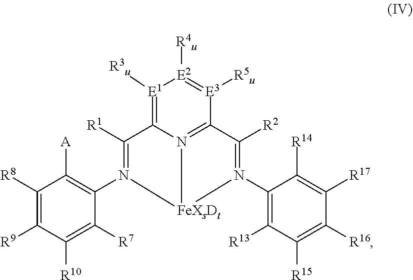

wherein:
A is chlorine, bromine, iodine, —CF₃ or —OR¹¹;
each of R¹ and R² is independently hydrogen, C₁-C₂₂-alkyl, C₂-C₂₂-alkenyl, C₆-C₂₂-aryl, arylalkyl where alkyl has from 1 to 10 carbon atoms and aryl has from 6 to 20 carbon atoms, or five-, six- or seven-membered heterocyclyl comprising at least one atom selected from the group consisting of N, P, O and S;
wherein each of R¹ and R² is optionally substituted by halogen, —NR¹¹₂, —OR¹¹ or —SiR¹²₃;
wherein R¹ optionally bonds with R³, and R² optionally bonds with R⁵, in each case to independently form a five-, six- or seven-membered ring;
R⁷ is a C₁-C₂₀ alkyl;
each of R³, R⁴, R⁵, R⁸, R⁹, R¹⁰, R¹⁵, R¹⁶, and R¹⁷ is independently hydrogen, C₁-C₂₂-alkyl, C₂-C₂₂-alkenyl, C₆-C₂₂-aryl, arylalkyl where alkyl has from 1 to 10 carbon atoms and aryl has from 6 to 20 carbon atoms, —NR¹¹₂, —OR¹¹, halogen, —SiR¹²₃ or five-, six- or seven-membered heterocyclyl comprising at least one atom selected from the group consisting of N, P, O, and S;
wherein R³, R⁴, R⁵, R⁷, R⁸, R⁹, R¹⁰, R¹⁵, R¹⁶, and R¹⁷ are optionally substituted by halogen, —NR¹¹₂, —OR¹¹ or —SiR¹²₃;
wherein v optionally bonds with R⁴, R⁴ optionally bonds with R⁵, R⁷ optionally bonds with R¹⁰, R¹⁰ optionally bonds with R⁹, R⁹ optionally bonds with R⁸, R¹⁷ optionally bonds with R¹⁶, and R¹⁶ optionally bonds with R¹⁵, in each case to independently form a five-, six- or seven-membered carbocyclic or heterocyclic ring, the heterocyclic ring comprising at least one atom from the group consisting of N, P, O and S;
R¹³ is C₁-C₂₀-alkyl bonded with the aryl ring via a primary or secondary carbon atom;
R¹⁴ is chlorine, bromine, iodine, —CF₃ or —OR¹¹, or C₁-C₂₀-alkyl bonded with the aryl ring; each R¹¹ is independently hydrogen, C₁-C₂₂-alkyl, C₂-C₂₂-alkenyl, C₆-C₂₂-aryl, arylalkyl where alkyl has from 1 to 10 carbon atoms and aryl has from 6 to 20 carbon atoms, or —SiR¹²₃, wherein R¹¹ is optionally substituted by halogen, or two R¹¹ radicals optionally bond to form a five- or six-membered ring;
each R¹² is independently hydrogen, C₁-C₂₂-alkyl, C₂-C₂₂-alkenyl, C₆-C₂₂-aryl, arylalkyl where alkyl has from 1 to 10 carbon atoms and aryl has from 6 to 20 carbon atoms, or two R¹² radicals optionally bond to form a five- or six-membered ring;
each of E¹, E², and E³ is independently carbon, nitrogen or phosphorus;
each u is independently 0 if E¹, E², and E³ is nitrogen or phosphorus and is 1 if E¹, E², and E³ is carbon;
each X is independently fluorine, chlorine, bromine, iodine, hydrogen, C₁-C₂₀-alkyl, C₂-C₁₀-alkenyl, C₆-C₂₀-aryl, arylalkyl where alkyl has from 1 to 10 carbon atoms and aryl has from 6 to 20 carbon atoms, —NR¹⁸₂, —OR¹⁸, —SR¹⁸, —SO₃R¹⁸, —OC(O)R¹⁸, —CN, —SCN, β-diketonate, —CO, —BF₄⁻, —PF₆⁻ or bulky non-coordinating anions, and the radicals X can be bonded with one another;
each R¹⁸ is independently hydrogen, C₁-C₂₀-alkyl, C₂-C₂₀-alkenyl, C₆-C₂₀-aryl, arylalkyl where alkyl has from 1 to 10 carbon atoms and aryl has from 6 to 20 carbon atoms, or —SiR¹⁹3, wherein R¹⁸ can be substituted by halogen or nitrogen- or oxygen-containing groups and two R¹⁸ radicals optionally bond to form a five- or six-membered ring;
each R¹⁹ is independently hydrogen, C₁-C₂₀-alkyl, C₂-C₂₀-alkenyl, C₆-C₂₀-aryl or arylalkyl where alkyl has from 1 to 10 carbon atoms and aryl has from 6 to 20 carbon atoms, wherein R¹⁹ can be substituted by halogen or nitrogen- or oxygen-containing groups or two R¹⁹ radicals optionally bond to form a five- or six-membered ring;
s is 1, 2, or 3;
D is a neutral donor; and
t is 0 to 2.

In another embodiment, the catalyst is a phenoxyimine compound represented by the formula (VII):

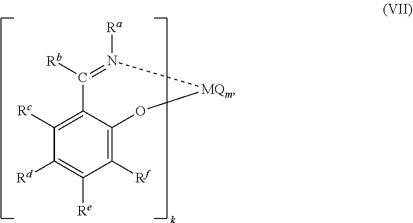

wherein M represents a transition metal atom selected from the groups 3 to 11 metals in the periodic table; k is an integer of 1 to 6; m is an integer of 1 to 6; R^{a} to R^{f} may be the same or different from one another and each represent a hydrogen atom, a halogen atom, a hydrocarbon group, a heterocyclic compound residue, an oxygen-containing group, a nitrogen-containing group, a boron-containing group, a sulfur-containing group, a phosphorus-containing group, a silicon-containing group, a germanium-containing group or a tin-containing group, among which 2 or more groups may be bound to each other to form a ring; when k is 2 or more, R^{a} groups, R^{b} groups, R^{c} groups, R^{d} groups, R^{e} groups, or R^{f} groups may be the same or different from one another, one group of R^{a} to R^{f} contained in one ligand and one group of R^{a} to R^{f} contained in another ligand may form a linking group or a single bond, and a heteroatom contained in R^{a} to R^{f} may coordinate with or bind to M; m is a number satisfying the valence of M; Q represents a hydrogen atom, a halogen atom, an oxygen atom, a hydrocarbon group, an oxygen-containing group, a sulfur-containing group, a nitrogen-containing group, a boron-containing group, an aluminum-containing group, a phosphorus-containing group, a halogen-containing group, a heterocyclic compound residue, a silicon-containing group, a germanium-containing group or a tin-containing group; when m is 2 or more, a plurality of groups represented by Q may be the same or different from one another, and a plurality of groups represented by Q may be mutually bound to form a ring.

In another embodiment, the catalyst is a bis(imino)pyridyl of the formula (VIII):

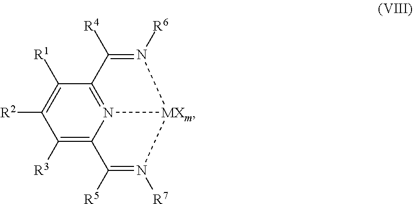

wherein:
M is Co or Fe; each X is an anion; n is 1, 2 or 3, so that the total number of negative charges on said anion or anions is equal to the oxidation state of a Fe or Co atom present in (VIII);
R¹, R² and R³ are each independently hydrogen, hydrocarbyl, substituted hydrocarbyl, or an inert functional group;
R⁴ and R⁵ are each independently hydrogen, hydrocarbyl, an inert functional group or substituted hydrocarbyl;
R⁶ is formula IX:

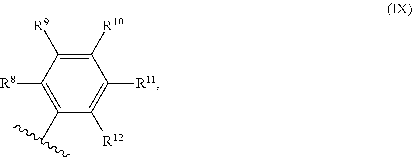

and R⁷ is formula X:

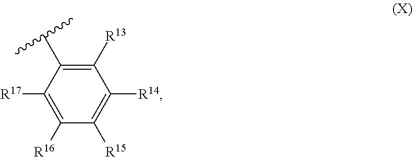

R⁸ and R¹³ are each independently hydrocarbyl, substituted hydrocarbyl or an inert functional group;
R⁹, R¹⁰, R¹¹, R¹⁴, R¹⁵ and R¹⁶ are each independently hydrogen, hydrocarbyl, substituted hydrocarbyl or an inert functional group;
R¹² and R¹⁷ are each independently hydrogen, hydrocarbyl, substituted hydrocarbyl or an inert functional group;
and provided that any two of R⁸, R⁹, R¹⁰, R¹¹, R¹², R¹³, R¹⁴, R¹⁵, R¹⁶ and R¹⁷ that are adjacent to one another, together may form a ring.

In at least one embodiment, the catalyst compound is represented by the formula (XI):

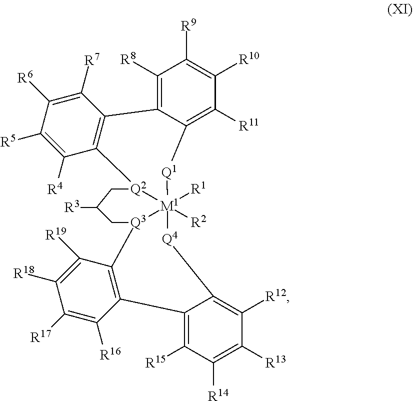

M¹ is selected from the group consisting of titanium, zirconium, hafnium, vanadium, niobium, tantalum, chromium, molybdenum and tungsten. In at least one embodiment, M¹ is zirconium.

Each of Q¹, Q², Q³, and Q⁴ is independently oxygen or sulfur. In at least one embodiment, at least one of Q¹, Q², Q³, and Q⁴ is oxygen, alternately all of Q¹, Q², Q³, and Q⁴ are oxygen.

R¹ and R² are independently hydrogen, halogen, hydroxyl, hydrocarbyl, or substituted hydrocarbyl (such as C₁-C₁₀ alkyl, C₁-C₁₀ alkoxy, C₆-C₂₀ aryl, C₆-C₁₀ aryloxy, C₂-C₁₀ alkenyl, C₂-C₄₀ alkenyl, C₇-C₄₀ arylalkyl, C₇-C₄₀ alkylaryl, C₈-C₄₀ arylalkenyl, or conjugated diene which is optionally substituted with one or more hydrocarbyl, tri(hydrocarbyl) silyl or tri(hydrocarbyl) silylhydrocarbyl, the diene having up to 30 atoms other than hydrogen). R¹ and R² can be a halogen selected from fluorine, chlorine, bromine, or iodine. Preferably, R¹ and R² are chlorine.

Alternatively, R¹ and R² may also be joined together to form an alkanediyl group or a conjugated C₄-C₄₀ diene ligand which is coordinated to M¹. R¹ and R² may also be identical or different conjugated dienes, optionally substituted with one or more hydrocarbyl, tri(hydrocarbyl) silyl or tri(hydrocarbyl) silylhydrocarbyl, the dienes having up to 30 atoms not counting hydrogen and/or forming a π-complex with M¹.

Exemplary groups suitable for R¹ and or R² can include 1,4-diphenyl, 1,3-butadiene, 1,3-pentadiene, 2-methyl 1,3-pentadiene, 2,4-hexadiene, 1-phenyl, 1,3-pentadiene, 1,4-dibenzyl, 1,3-butadiene, 1,4-ditolyl-1,3-butadiene, 1,4-bis(trimethylsilyl)-1,3-butadiene, and 1,4-dinaphthyl-1,3-butadiene. R¹ and R² can be identical and are C₁-C₃ alkyl or alkoxy, C₆-C₁₀ aryl or aryloxy, C₂-C₄ alkenyl, C₇-C₁₀ arylalkyl, C₇-C₁₂ alkylaryl, or halogen.

Each of R⁴, R⁵, R⁶, R⁷, R⁸, R⁹, R¹⁰, R¹¹, R¹², R¹³, R¹⁴, R¹⁵, R¹⁶, R¹⁷, R¹⁸, and R¹⁹ is independently hydrogen, halogen, C₁-C₄₀ hydrocarbyl or C₁-C₄₀ substituted hydrocarbyl (such as C₁-C₁₀ alkyl, C₁-C₁₀ alkoxy, C₆-C₂₀ aryl, C₆-C₁₀ aryloxy, C₂-C₁₀ alkenyl, C₂-C₄₀ alkenyl, C₇-C₄₀ arylalkyl, C₇-C₄₀ alkylaryl, C₈-C₄₀ arylalkenyl, or conjugated diene which is optionally substituted with one or more hydrocarbyl, tri(hydrocarbyl) silyl or tri(hydrocarbyl) silylhydrocarbyl, the diene having up to 30 atoms other than hydrogen), —NR′₂, —SR′, —OR, —OSiR′₃, —PR′₂, where each R′ is hydrogen, halogen, C₁-C₁₀ alkyl, or C₆-C₁₀ aryl, or one or more of R⁴ and R⁵, R⁵ and R⁶, R⁶ and R⁷, R⁸ and R⁹, R⁹ and R¹⁰, R¹⁰ and R¹¹, R¹² and R¹³, R¹³ and R¹⁴, R¹⁴ and R¹⁵, R¹⁶ and R¹⁷, R¹⁷ and R¹⁸, and R¹⁸ and R¹⁹ are joined to form a saturated ring, unsaturated ring, substituted saturated ring, or substituted unsaturated ring. In at least one embodiment, C₁-C₄₀ hydrocarbyl is selected from methyl, ethyl, propyl, n-propyl, isopropyl, n-butyl, isobutyl, sec-butyl, n-pentyl, isopentyl, sec-pentyl, n-hexyl, isohexyl, sec-hexyl, n-heptyl, isoheptyl, sec-heptyl, n-octyl, isooctyl, sec-octyl, n-nonyl, isononyl, sec-nonyl, n-decyl, isodecyl, and sec-decyl. Preferably, R¹¹ and R¹² are C₆-C₁₀ aryl such as phenyl or naphthyl optionally substituted with C₁-C₄₀ hydrocarbyl, such as C₁-C₁₀ hydrocarbyl. Preferably, R⁶ and R¹⁷ are C₁-40 alkyl, such as C₁-C₁₀ alkyl.

In at least one embodiment, each of R⁴, R⁵, R⁶, R⁷, R⁸, R⁹, R¹⁰, R¹¹, R¹², R¹³, R¹⁴, R¹⁵, R¹⁶, R¹⁷, R¹⁸, and R¹⁹ is independently hydrogen or C₁-C₄₀ hydrocarbyl. In at least one embodiment, C₁-C₄₀ hydrocarbyl is selected from methyl, ethyl, propyl, n-propyl, isopropyl, n-butyl, isobutyl, sec-butyl, n-pentyl, isopentyl, sec-pentyl, n-hexyl, isohexyl, sec-hexyl, n-heptyl, isoheptyl, sec-heptyl, n-octyl, isooctyl, sec-octyl, n-nonyl, isononyl, sec-nonyl, n-decyl, isodecyl, and sec-decyl. Preferably, each of R⁶ and R¹⁷ is C₁-C₄₀ hydrocarbyl and R⁴, R⁵, R⁷, R⁸, R⁹, R¹⁰, R¹³, R¹⁴, R¹⁵, R¹⁶, R¹⁸, and R¹⁹ is hydrogen. In at least one embodiment, C₁-C₄₀ hydrocarbyl is selected from methyl, ethyl, propyl, n-propyl, isopropyl, n-butyl, isobutyl, sec-butyl, n-pentyl, isopentyl, sec-pentyl, n-hexyl, isohexyl, sec-hexyl, n-heptyl, isoheptyl, sec-heptyl, n-octyl, isooctyl, sec-octyl, n-nonyl, isononyl, sec-nonyl, n-decyl, isodecyl, and sec-decyl.

R³ is a C₁-C₄₀ unsaturated alkyl or substituted C₁-C₄₀ unsaturated alkyl (such as C₁-C₁₀ alkyl, C₁-C₁₀ alkoxy, C₆-C₂₀ aryl, C₆-C₁₀ aryloxy, C₂-C₁₀ alkenyl, C₂-C₄₀ alkenyl, C₇-C₄₀ arylalkyl, C₇-C₄₀ alkylaryl, C₈-C₄₀ arylalkenyl, or conjugated diene which is optionally substituted with one or more hydrocarbyl, tri(hydrocarbyl) silyl or tri(hydrocarbyl) silylhydrocarbyl, the diene having up to 30 atoms other than hydrogen).

Preferably, R³ is a hydrocarbyl comprising a vinyl moiety. As used herein, “vinyl” and “vinyl moiety” are used interchangeably and include a terminal alkene, e.g., represented by the structure

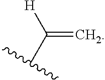 [SMILES: *C=C]

Hydrocarbyl of R³ may be further substituted (such as C₁-C₁₀ alkyl, C₁-C₁₀ alkoxy, C₆-C₂₀ aryl, C₆-C₁₀ aryloxy, C₂-C₁₀ alkenyl, C₂-C₄₀ alkenyl, C₇-C₄₀ arylalkyl, C₇-C₄₀ alkylaryl, C₈-C₄₀ arylalkenyl, or conjugated diene which is optionally substituted with one or more hydrocarbyl, tri(hydrocarbyl) silyl or tri(hydrocarbyl) silylhydrocarbyl, the diene having up to 30 atoms other than hydrogen). Preferably, R³ is C₁-C₄₀ unsaturated alkyl that is vinyl or substituted C₁-C₄₀ unsaturated alkyl that is vinyl. R³ can be represented by the structure —R′CH═CH₂ where R′ is C₁-C₄₀ hydrocarbyl or C₁-C₄₀ substituted hydrocarbyl (such as C₁-C₁₀ alkyl, C₁-C₁₀ alkoxy, C₆-C₂₀ aryl, C₆-C₁₀ aryloxy, C₂-C₁₀ alkenyl, C₂-C₄₀ alkenyl, C₇-C₄₀ arylalkyl, C₇-C₄₀ alkylaryl, C₈-C₄₀ arylalkenyl, or conjugated diene which is optionally substituted with one or more hydrocarbyl, tri(hydrocarbyl) silyl or tri(hydrocarbyl) silylhydrocarbyl, the diene having up to 30 atoms other than hydrogen). In at least one embodiment, C₁-C₄₀ hydrocarbyl is selected from methyl, ethyl, propyl, n-propyl, isopropyl, n-butyl, isobutyl, sec-butyl, n-pentyl, isopentyl, sec-pentyl, n-hexyl, isohexyl, sec-hexyl, n-heptyl, isoheptyl, sec-heptyl, n-octyl, isooctyl, sec-octyl, n-nonyl, isononyl, sec-nonyl, n-decyl, isodecyl, and sec-decyl.

In at least one embodiment, R³ is 1-propenyl, 1-butenyl, 1-pentenyl, 1-hexenyl, 1-heptenyl, 1-octenyl, 1-nonenyl, or 1-decenyl.

In at least one embodiment, the catalyst is a Group 15-containing metal compound represented by Formulas (XII) or (XIII):

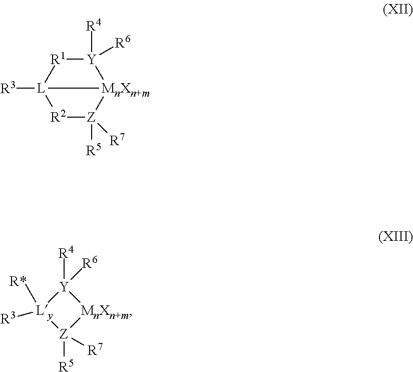

wherein M is a Group 3 to 12 transition metal or a Group 13 or 14 main group metal, a Group 4, 5, or 6 metal. In many embodiments, M is a Group 4 metal, such as zirconium, titanium, or hafnium. Each X is independently a leaving group, such as an anionic leaving group. The leaving group may include a hydrogen, a hydrocarbyl group, a heteroatom, a halogen, or an alkyl; y is 0 or 1 (when y is 0 group L′ is absent). The term ‘n’ is the oxidation state of M. In various embodiments, n is +3, +4, or +5. In many embodiments, n is +4. The term ‘m’ represents the formal charge of the YZL or the YZL′ ligand, and is 0, −1, −2 or −3 in various embodiments. In many embodiments, m is −2. L is a Group 15 or 16 element, such as nitrogen or oxygen; L′ is a Group 15 or 16 element or Group 14 containing group, such as carbon, silicon or germanium. Y is a Group 15 element, such as nitrogen or phosphorus. In many embodiments, Y is nitrogen. Z is a Group 15 element, such as nitrogen or phosphorus. In many embodiments, Z is nitrogen. R¹ and R² are, independently, a C₁ to C₂₀ hydrocarbon group, a heteroatom containing group having up to twenty carbon atoms, silicon, germanium, tin, lead, or phosphorus. In many embodiments, R¹ and R² are a C₂ to C₂₀ alkyl, aryl or aralkyl group, such as a C₂ to C₂₀ linear, branched or cyclic alkyl group, or a C₂ to C₂₀ hydrocarbon group. R¹ and R² may also be interconnected to each other. R³ may be absent or may be a hydrocarbon group, a hydrogen, a halogen, a heteroatom containing group. In many embodiments, R³ is absent, for example, if L is an oxygen, or a hydrogen, or a linear, cyclic, or branched alkyl group having 1 to 20 carbon atoms. R⁴ and R⁵ are independently an alkyl group, an aryl group, substituted aryl group, a cyclic alkyl group, a substituted cyclic alkyl group, a cyclic aralkyl group, a substituted cyclic aralkyl group, or multiple ring system, often having up to 20 carbon atoms. In many embodiments, R⁴ and R⁵ have between 3 and 10 carbon atoms, or are a C₁ to C₂₀ hydrocarbon group, a C₁ to C₂₀ aryl group or a C₁ to C₂₀ aralkyl group, or a heteroatom containing group. R⁴ and R⁵ may be interconnected to each other. R⁶ and R⁷ are independently absent, hydrogen, an alkyl group, halogen, heteroatom, or a hydrocarbyl group, such as a linear, cyclic or branched alkyl group having 1 to 20 carbon atoms. In many embodiments, R⁶ and R⁷ are absent. R* may be absent, or may be a hydrogen, a Group 14 atom containing group, a halogen, or a heteroatom containing group.

By “formal charge of the YZL or YZL′ ligand,” it is meant the charge of the entire ligand absent the metal and the leaving groups X. By “R¹ and R² may also be interconnected” it is meant that R¹ and R² may be directly bound to each other or may be bound to each other through other groups. By “R⁴ and R⁵ may also be interconnected” it is meant that R⁴ and R⁵ may be directly bound to each other or may be bound to each other through other groups. An alkyl group may be linear, branched alkyl radicals, alkenyl radicals, alkynyl radicals, cycloalkyl radicals, aryl radicals, acyl radicals, aroyl radicals, alkoxy radicals, aryloxy radicals, alkylthio radicals, dialkylamino radicals, alkoxycarbonyl radicals, aryloxycarbonyl radicals, carbomoyl radicals, alkyl- or dialkyl-carbamoyl radicals, acyloxy radicals, acylamino radicals, aroylamino radicals, straight, branched or cyclic, alkylene radicals, or combination thereof. An aralkyl group is defined to be a substituted aryl group.

In one or more embodiments, R⁴ and R⁵ are independently a group represented by structure (XIV):

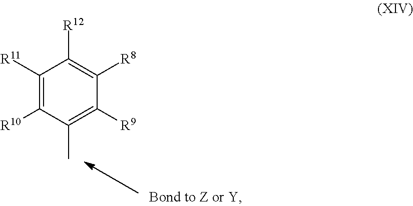

wherein R⁸ to R¹² are each independently hydrogen, a C₁ to C₄₀ alkyl group, a halide, a heteroatom, a heteroatom containing group containing up to 40 carbon atoms. In many embodiments, R⁸ to R¹² are a C₁ to C₂₀ linear or branched alkyl group, such as a methyl, ethyl, propyl, or butyl group. Any two of the R groups may form a cyclic group and/or a heterocyclic group. The cyclic groups may be aromatic. In one embodiment R⁹, R¹⁰ and R¹² are independently a methyl, ethyl, propyl, or butyl group (including all isomers). In another embodiment, R⁹, R¹⁰ and R¹² are methyl groups, and R⁸ and R¹¹ are hydrogen.

In one or more embodiments, R⁴ and R⁵ are both a group represented by structure (XV):

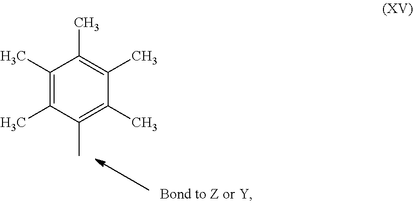

wherein M is a Group 4 metal, such as zirconium, titanium, or hafnium. In at least one embodiment, M is zirconium. Each of L, Y, and Z may be a nitrogen. Each of R¹ and R² may be —CH₂—CH₂—. R³ may be hydrogen, and R⁶ and R⁷ may be absent.

In preferred embodiments, the catalyst compounds described in PCT/US2018/051345, filed Sep. 17, 2018 may be used with the activators described herein, particularly the catalyst compounds described at Page 16 to Page 32 of the application as filed.

In some embodiments, a co-activator is combined with the catalyst compound (such as halogenated catalyst compounds described above) to form an alkylated catalyst compound. Organoaluminum compounds which may be utilized as co-activators include, for example, trialkyl aluminum compounds, such as trimethylaluminum, triethylaluminum, triisobutylaluminum, tri-n-hexylaluminum, tri-n-octylaluminum, and the like, or alumoxanes.

In some embodiments, two or more different catalyst compounds are present in the catalyst system used herein. In some embodiments, two or more different catalyst compounds are present in the reaction zone where the process(es) described herein occur. When two transition metal compound based catalysts are used in one reactor as a mixed catalyst system, the two transition metal compounds are preferably chosen such that the two are compatible. A simple screening method such as by ¹H or ¹³C NMR, known to those of ordinary skill in the art, can be used to determine which transition metal compounds are compatible. It is preferable to use the same activator for the transition metal compounds, however, two different activators can be used in combination. If one or more transition metal compounds contain an anionic ligand as a leaving group which is not a hydride, hydrocarbyl, or substituted hydrocarbyl, then the alumoxane or other alkyl aluminum is typically contacted with the transition metal compounds prior to addition of the non-coordinating anion activator.

The two transition metal compounds (pre-catalysts) may be used in any ratio. Preferred molar ratios of (A) transition metal compound to (B) transition metal compound fall within the range of (A:B) 1:1000 to 1000:1, alternatively 1:100 to 500:1, alternatively 1:10 to 200:1, alternatively 1:1 to 100:1, and alternatively 1:1 to 75:1, and alternatively 5:1 to 50:1. The particular ratio chosen will depend on the exact pre-catalysts chosen, the method of activation, and the end product desired. In a particular embodiment, when using the two pre-catalysts, where both are activated with the same activator, useful mole percents, based upon the molecular weight of the pre-catalysts, are 10 to 99.9% A to 0.1 to 90% B, alternatively 25 to 99% A to 0.5 to 50% B, alternatively 50 to 99% A to 1 to 25% B, and alternatively 75 to 99% A to 1 to 10% B.

### Support Materials

In embodiments herein, the catalyst system may comprise a support material. In at least one embodiment, the support material is a porous support material, for example, talc, or inorganic oxides. Other support materials include zeolites, clays, organoclays, or any other suitable organic or inorganic support material and the like, or mixtures thereof.

In at least one embodiment, the support material is an inorganic oxide. Suitable inorganic oxide materials for use in catalyst systems herein include Groups 2, 4, 13, and 14 metal oxides, such as silica, alumina, and mixtures thereof. Other inorganic oxides that may be employed either alone or in combination with the silica, or alumina are magnesia, titania, zirconia, and the like. Other suitable support materials, however, can be used, for example, functionalized polyolefins, such as polypropylene. Supports include magnesia, titania, zirconia, montmorillonite, phyllosilicate, zeolites, talc, clays, and the like. Also, combinations of these support materials may be used, for example, silica-chromium, silica-alumina, silica-titania, and the like. Support materials include Al₂O₃, ZrO₂, SiO₂, SiO₂/Al₂O₃, SiO₂/TiO₂, silica clay, silicon oxide/clay, or mixtures thereof.

The support material, such as an inorganic oxide, can have a surface area of from 10 m²/g to 700 m²/g, pore volume in the range of from 0.1 cc/g to 4.0 cc/g and average particle size in the range of from 5 m to 500 m. In at least one embodiment, the surface area of the support material is in the range of from 50 m²/g to 500 m²/g, pore volume of from 0.5 cc/g to 3.5 cc/g and average particle size of from 10 m to 200 m. In at least one embodiment, the surface area of the support material is in the range is from 100 m²/g to 400 m²/g, pore volume from 0.8 cc/g to 3.0 cc/g and average particle size is from 5 m to 100 m. The average pore size of the support material useful in the present disclosure is in the range of from 10 Å to 1000 Å, such as 50 Å to 500 Å, such as 75 Å to 350 Å. In some embodiments, the support material is a high surface area, amorphous silica (surface area=300 m²/gm; pore volume of 1.65 cm³/gm). Exemplary silicas are marketed under the tradenames of DAVISON 952 or DAVISON 955 by the Davison Chemical Division of W.R. Grace and Company. In other embodiments DAVISON 948 is used.

The support material should be dry, that is, substantially free of absorbed water. Drying of the support material can be effected by heating or calcining at 100° C. to 1,000° C., such as at least about 600° C. When the support material is silica, it is heated to at least 200° C., such as 200° C. to 850° C., such as at about 600° C.; and for a time of 1 minute to about 100 hours, from 12 hours to 72 hours, or from 24 hours to 60 hours. The calcined support material should have at least some reactive hydroxyl (OH) groups to produce supported catalyst systems of the present disclosure. The calcined support material is then contacted with at least one polymerization catalyst comprising at least one catalyst compound and an activator.

The support material, having reactive surface groups, typically hydroxyl groups, is slurried in a non-polar solvent and the resulting slurry is contacted with a solution of a catalyst compound and an activator. In some embodiments, the slurry of the support material is first contacted with the activator for a period of time in the range of from 0.5 hours to 24 hours, from 2 hours to 16 hours, or from 4 hours to 8 hours. The solution of the catalyst compound is then contacted with the isolated support/activator. In some embodiments, the supported catalyst system is generated in situ. In at least one embodiment, the slurry of the support material is first contacted with the catalyst compound for a period of time in the range of from 0.5 hours to 24 hours, from 2 hours to 16 hours, or from 4 hours to 8 hours. The slurry of the supported catalyst compound is then contacted with the activator solution.

The mixture of the catalyst, activator and support is heated to 0° C. to 70° C., such as to 23° C. to 60° C., such as at room temperature. Contact times typically range from 0.5 hours to 24 hours, from 2 hours to 16 hours, or from 4 hours to 8 hours.

Suitable non-polar solvents are materials in which all of the reactants used herein, e.g., the activator, and the catalyst compound, are at least partially soluble and which are liquid at room temperature. Non-limiting example non-polar solvents are alkanes, such as isopentane, hexane, n-heptane, octane, nonane, and decane, cycloalkanes, such as cyclohexane, aromatics, such as benzene, toluene, and ethylbenzene.

In at least one embodiment, the support material comprises a support material treated with an electron-withdrawing anion. The support material can be silica, alumina, silica-alumina, silica-zirconia, alumina-zirconia, aluminum phosphate, heteropolytungstates, titania, magnesia, boria, zinc oxide, mixed oxides thereof, or mixtures thereof; and the electron-withdrawing anion is selected from fluoride, chloride, bromide, phosphate, triflate, bisulfate, sulfate, or any combination thereof.

The electron-withdrawing component used to treat the support material can be any component that increases the Lewis or Brønsted acidity of the support material upon treatment (as compared to the support material that is not treated with at least one electron-withdrawing anion). In at least one embodiment, the electron-withdrawing component is an electron-withdrawing anion derived from a salt, an acid, or other compound, such as a volatile organic compound, that serves as a source or precursor for that anion. Electron-withdrawing anions can be sulfate, bisulfate, fluoride, chloride, bromide, iodide, fluorosulfate, fluoroborate, phosphate, fluorophosphate, trifluoroacetate, triflate, fluorozirconate, fluorotitanate, phospho-tungstate, or mixtures thereof, or combinations thereof. An electron-withdrawing anion can be fluoride, chloride, bromide, phosphate, triflate, bisulfate, or sulfate, or any combination thereof, at least one embodiment of this disclosure. In at least one embodiment, the electron-withdrawing anion is sulfate, bisulfate, fluoride, chloride, bromide, iodide, fluorosulfate, fluoroborate, phosphate, fluorophosphate, trifluoroacetate, triflate, fluorozirconate, fluorotitanate, or combinations thereof.

Thus, for example, the support material suitable for use in the catalyst systems of the present disclosure can be one or more of fluorided alumina, chlorided alumina, bromided alumina, sulfated alumina, fluorided silica-alumina, chlorided silica-alumina, bromided silica-alumina, sulfated silica-alumina, fluorided silica-zirconia, chlorided silica-zirconia, bromided silica-zirconia, sulfated silica-zirconia, fluorided silica-titania, fluorided silica-coated alumina, sulfated silica-coated alumina, phosphated silica-coated alumina, or combinations thereof. In at least one embodiment, the activator-support can be, or can comprise, fluorided alumina, sulfated alumina, fluorided silica-alumina, sulfated silica-alumina, fluorided silica-coated alumina, sulfated silica-coated alumina, phosphated silica-coated alumina, or combinations thereof. In another embodiment, the support material includes alumina treated with hexafluorotitanic acid, silica-coated alumina treated with hexafluorotitanic acid, silica-alumina treated with hexafluorozirconic acid, silica-alumina treated with trifluoroacetic acid, fluorided boria-alumina, silica treated with tetrafluoroboric acid, alumina treated with tetrafluoroboric acid, alumina treated with hexafluorophosphoric acid, or combinations thereof. Further, any of these activator-supports optionally can be treated with a metal ion.

Nonlimiting examples of cations suitable for use in the present disclosure in the salt of the electron-withdrawing anion include ammonium, trialkyl ammonium, tetraalkyl ammonium, tetraalkyl phosphonium, H+, [H(OEt₂)₂]+, or combinations thereof.

Further, combinations of one or more different electron-withdrawing anions, in varying proportions, can be used to tailor the specific acidity of the support material to a desired level. Combinations of electron-withdrawing components can be contacted with the support material simultaneously or individually, and in any order that provides a desired chemically-treated support material acidity. For example, in at least one embodiment, two or more electron-withdrawing anion source compounds in two or more separate contacting steps.

In at least one embodiment of the present disclosure, one example of a process by which a chemically-treated support material is prepared is as follows: a selected support material, or combination of support materials, can be contacted with a first electron-withdrawing anion source compound to form a first mixture; such first mixture can be calcined and then contacted with a second electron-withdrawing anion source compound to form a second mixture; the second mixture can then be calcined to form a treated support material. In such a process, the first and second electron-withdrawing anion source compounds can be either the same or different compounds.

The method by which the oxide is contacted with the electron-withdrawing component, typically a salt or an acid of an electron-withdrawing anion, can include gelling, co-gelling, impregnation of one compound onto another, or combinations thereof. Following a contacting method, the contacted mixture of the support material, electron-withdrawing anion, and optional metal ion, can be calcined.

According to another embodiment of the present disclosure, the support material can be treated by a process comprising: (i) contacting a support material with a first electron-withdrawing anion source compound to form a first mixture; (ii) calcining the first mixture to produce a calcined first mixture; (iii) contacting the calcined first mixture with a second electron-withdrawing anion source compound to form a second mixture; and (iv) calcining the second mixture to form the treated support material.

### Polymer Processes

In embodiments herein, the present disclosure provides polymerization processes where monomer (such as propylene or ethylene), and optionally comonomer, are contacted with a catalyst system comprising an activator and at least one catalyst compound, as described above. The catalyst compound and activator may be combined in any order, and are combined typically prior to contacting with the monomer.

In at least one embodiment, a polymerization process includes a) contacting one or more olefin monomers with a catalyst system comprising: i) an activator and ii) a catalyst compound of the present disclosure. The activator is a non-coordination anion activator. The one or more olefin monomers may be propylene and/or ethylene and the polymerization process further comprises heating the one or more olefin monomers and the catalyst system to 70° C. or more to form propylene polymers or ethylene polymers, such as propylene polymers.

Monomers useful herein include substituted or unsubstituted C₂ to C₄₀ alpha olefins, such as C₂ to C₂₀ alpha olefins, such as C₂ to C₁₂ alpha olefins, such as ethylene, propylene, butene, pentene, hexene, heptene, octene, nonene, decene, undecene, dodecene and isomers thereof. In at least one embodiment, the monomer comprises propylene and an optional comonomers comprising one or more propylene or C₄ to C₄₀ olefins, such as C₄ to C₂₀ olefins, such as C₆ to C₁₂ olefins. The C₄ to C₄₀ olefin monomers may be linear, branched, or cyclic. The C₄ to C₄₀ cyclic olefins may be strained or unstrained, monocyclic or polycyclic, and may optionally include heteroatoms and/or one or more functional groups. In at least one embodiment, the monomer comprises propylene and an optional comonomers comprising one or more C₃ to C₄₀ olefins, such as C₄ to C₂₀ olefins, such as C₆ to C₁₂ olefins. The C₃ to C₄₀ olefin monomers may be linear, branched, or cyclic. The C₃ to C₄₀ cyclic olefins may be strained or unstrained, monocyclic or polycyclic, and may optionally include heteroatoms and/or one or more functional groups.

Exemplary C₂ to C₄₀ olefin monomers and optional comonomers include propylene, propylene, butene, pentene, hexene, heptene, octene, nonene, decene, undecene, dodecene, norbornene, norbomadiene, dicyclopentadiene, cyclopentene, cycloheptene, cyclooctene, cyclooctadiene, cyclododecene, 7-oxanorbomene, 7-oxanorbomadiene, substituted derivatives thereof, and isomers thereof, such as hexene, heptene, octene, nonene, decene, dodecene, cyclooctene, 1,5-cyclooctadiene, 1-hydroxy-4-cyclooctene, 1-acetoxy-4-cyclooctene, 5-methylcyclopentene, cyclopentene, dicyclopentadiene, norbomene, norbomadiene, and their respective homologs and derivatives, such as norbomene, norbomadiene, and dicyclopentadiene.

In at least one embodiment, one or more dienes are present in the polymer produced herein at up to 10 wt %, such as at 0.00001 to 1.0 wt %, such as 0.002 to 0.5 wt %, such as 0.003 to 0.2 wt %, based upon the total weight of the composition. In some embodiments, 500 ppm or less of diene is added to the polymerization, such as 400 ppm or less, such as 300 ppm or less. In other embodiments at least 50 ppm of diene is added to the polymerization, or 100 ppm or more, or 150 ppm or more.

Diene monomers include any hydrocarbon structure, such as C₄ to C₃₀, having at least two unsaturated bonds, wherein at least two of the unsaturated bonds are readily incorporated into a polymer by either a stereospecific or a non-stereospecific catalyst(s). The diene monomers can be selected from alpha, omega-diene monomers (i.e. di-vinyl monomers). The diolefin monomers are linear di-vinyl monomers, such as those containing from 4 to 30 carbon atoms. Examples of dienes include butadiene, pentadiene, hexadiene, heptadiene, octadiene, nonadiene, decadiene, undecadiene, dodecadiene, tridecadiene, tetradecadiene, pentadecadiene, hexadecadiene, heptadecadiene, octadecadiene, nonadecadiene, icosadiene, heneicosadiene, docosadiene, tricosadiene, tetracosadiene, pentacosadiene, hexacosadiene, heptacosadiene, octacosadiene, nonacosadiene, triacontadiene, 1,6-heptadiene, 1,7-octadiene, 1,8-nonadiene, 1,9-decadiene, 1,10-undecadiene, 1,11-dodecadiene, 1,12-tridecadiene, 1,13-tetradecadiene, and low molecular weight polybutadienes (Mw less than 1000 g/mol). Cyclic dienes include cyclopentadiene, vinylnorbomene, norbomadiene, ethylidene norbornene, divinylbenzene, dicyclopentadiene or higher ring containing diolefins with or without substituents at various ring positions.

Polymerization processes of the present disclosure can be carried out in any suitable manner. Any suitable suspension, homogeneous, bulk, solution, slurry, or gas phase polymerization process can be used. Such processes can be run in a batch, semi-batch, or continuous mode. Homogeneous polymerization processes and slurry processes can be performed. (A useful homogeneous polymerization process is one where at least 90 wt % of the product is soluble in the reaction media.) A bulk homogeneous process can be used. (An example bulk process is one where monomer concentration in all feeds to the reactor is 70 volume % or more.) Alternately, no solvent or diluent is present or added in the reaction medium, (except for the small amounts used as the carrier for the catalyst system or other additives, or amounts typically found with the monomer; e.g., propane in propylene). In at least one embodiment, the process is a slurry polymerization process. As used herein the term “slurry polymerization process” means a polymerization process where a supported catalyst is employed and monomers are polymerized on the supported catalyst particles. At least 95 wt % of polymer products derived from the supported catalyst are in granular form as solid particles (not dissolved in the diluent).

Suitable diluents/solvents for polymerization include non-coordinating, inert liquids. Examples include straight and branched-chain hydrocarbons, such as isobutane, butane, pentane, isopentane, hexanes, isohexane, heptane, octane, dodecane, and mixtures thereof; cyclic and alicyclic hydrocarbons, such as cyclohexane, cycloheptane, methylcyclohexane, methylcycloheptane, and mixtures thereof, such as can be found commercially (Isopar™); perhalogenated hydrocarbons, such as perfluorinated C₄-C₁₀ alkanes, chlorobenzene, and aromatic and alkylsubstituted aromatic compounds, such as benzene, toluene, mesitylene, and xylene. Suitable solvents also include liquid olefins which may act as monomers or comonomers including ethylene, propylene, 1-butene, 1-hexene, 1-pentene, 3-methyl-1-pentene, 4-methyl-1-pentene, 1-octene, 1-decene, and mixtures thereof. In at least one embodiment, the solvent is not aromatic, such that aromatics are present in the solvent at less than 1 wt %, such as less than 0.5 wt %, such as less than 0 wt % based upon the weight of the solvents.

In at least one embodiment, the feed concentration of the monomers and comonomers for the polymerization is 60 vol % solvent or less, such as 40 vol % or less, such as 20 vol % or less, based on the total volume of the feedstream. The polymerization can be performed in a bulk process.

Polymerizations can be performed at any temperature and/or pressure suitable to obtain the desired polymers, such as ethylene and or propylene polymers. Typical temperatures and/or pressures include a temperature in the range of from 0° C. to 300° C., such as 20° C. to 200° C., such as 35° C. to 150° C., such as 40° C. to 120° C., such as 45° C. to 80° C., for example about 74° C., and at a pressure in the range of from 0.35 MPa to 10 MPa, such as 0.45 MPa to 6 MPa, such as 0.5 MPa to 4 MPa.

In a typical polymerization, the run time of the reaction is up to 300 minutes, such as in the range of from 5 to 250 minutes, such as 10 to 120 minutes.

In at least one embodiment, hydrogen is present in the polymerization reactor at a partial pressure of 0.001 to 50 psig (0.007 to 345 kPa), such as from 0.01 to 25 psig (0.07 to 172 kPa), such as 0.1 to 10 psig (0.7 to 70 kPa).

In at least one embodiment, the activity of the catalyst is from 50 gP/mmolCat/hour to 200,000 gP/mmolCat/hr, such as from 10,000 gP/mmolCat/hr to 150,000 gP/mmolCat/hr, such as from 40,000 gP/mmolCat/hr to 100,000 gP/mmolCat/hr, such as about 50,000 gP/mmolCat/hr or more, such as 70,000 gP/mmolCat/hr or more. In at least one embodiment, the conversion of olefin monomer is at least 10%, based upon polymer yield and the weight of the monomer entering the reaction zone, such as 20% or more, such as 30% or more, such as 50% or more, such as 80% or more.

In at least one embodiment, a catalyst system of the present disclosure is capable of producing a polyolefin. In at least one embodiment, a polyolefin is a homopolymer of ethylene or propylene or a copolymer of ethylene such as a copolymer of ethylene having from 0.1 to 25 wt % (such as from 0.5 to 20 wt %, such as from 1 to 15 wt %, such as from 5 to 17 wt %) of ethylene with the remainder balance being one or more C₃ to C₂₀ olefin comonomers (such as C₃ to C₁₂ alpha-olefin, such as propylene, butene, hexene, octene, decene, dodecene, such as propylene, butene, hexene, octene). A polyolefin can be a copolymer of propylene such as a copolymer of propylene having from 0.1 to 25 wt % (such as from 0.5 to 20 wt %, such as from 1 to 15 wt %, such as from 3 to 10 wt %) of propylene and from 99.9 to 75 wt % of one or more of C₂ or C₄ to C₂₀ olefin comonomer (such as ethylene or C₄ to C₁₂ alpha-olefin, such as butene, hexene, octene, decene, dodecene, such as ethylene, butene, hexene, octene).

In at least one embodiment, a catalyst system of the present disclosure is capable of producing polyolefins, such as polypropylene (e.g., iPP) or ethylene-octene copolymers, having an Mw from 40,000 to 1,500,000, such as from 70,000 to 1,000,000, such as from 90,000 to 500,000, such as from 90,000 to 250,000, such as from 90,000 to 200,000, such as from 90,000 to 110,000.

In at least one embodiment, a catalyst system of the present disclosure is capable of producing polyolefins, such as polypropylene (e.g., iPP) or ethylene-octene copolymers, having an Mn from 5,000 to 1,000,000, such as from 20,000 to 160,000, such as from 30,000 to 70,000, such as from 40,000 to 70,000. In at least one embodiment, a catalyst system of the present disclosure is capable of producing propylene polymers having an Mw/Mn value from 1 to 10, such as from 1.5 to 9, such as from 2 to 7, such as from 2 to 4, such as from 2.5 to 3, for example about 2.

In at least one embodiment, a catalyst system of the present disclosure is capable of producing polyolefins, such as polypropylene (e.g., iPP) or ethylene-octene copolymers, having a melt temperature (Tm) of from 100° C. to 150° C., such as 110° C. to 140° C., such as 120° C. to 135° C., such as 130° C. to 135° C.

In at least one embodiment, little or no activator is used in the process to produce the polymers. Activator can be present at zero mol %, alternatively the activator is present at a molar ratio of aluminum to transition metal less than 500:1, such as less than 300:1, such as less than 100:1, such as less than 1:1.

In at least one embodiment, little or no scavenger is used in the process to produce the propylene polymer. Scavenger (such as trialkyl aluminum) can be present at zero mol %, alternately the scavenger is present at a molar ratio of scavenger metal to transition metal of less than 100:1, such as less than 50:1, such as less than 15:1, such as less than 10:1.

In at least one embodiment, the polymerization: 1) is conducted at temperatures of 0 to 300° C. (such as 25 to 150° C., such as 40 to 120° C., such as 70 to 110° C., such as 85 to 100° C.); 2) is conducted at a pressure of atmospheric pressure to 10 MPa (such as 0.35 to 10 MPa, such as from 0.45 to 6 MPa, such as from 0.5 to 4 MPa); 3) is conducted in an aliphatic hydrocarbon solvent (such as isobutane, butane, pentane, isopentane, hexanes, isohexane, heptane, octane, dodecane, and mixtures thereof; cyclic and alicyclic hydrocarbons, such as cyclohexane, cycloheptane, methylcyclohexane, methylcycloheptane, and mixtures thereof, where aromatics are present in the solvent at less than 1 wt %, such as less than 0.5 wt %, such as at 0 wt % based upon the weight of the solvents); and 4) the productivity of the catalyst compound is at least 30,000 gP/mmolCat/hr (such as at least 50,000 gP/mmolCat/hr, such as at least 60,000 gP/mmolCat/hr, such as at least 80,000 gP/mmolCat/hr, such as at least 100,000 gP/mmolCat/hr).

In at least one embodiment, the catalyst system used in the polymerization comprises no more than one catalyst compound. A “reaction zone” also referred to as a “polymerization zone” is a vessel where polymerization takes place, for example a batch reactor. When multiple reactors are used in either series or parallel configuration, each reactor is considered as a separate polymerization zone. For a multi-stage polymerization in both a batch reactor and a continuous reactor, each polymerization stage is considered as a separate polymerization zone. In at least one embodiment, the polymerization occurs in one reaction zone.

Other additives may also be used in the polymerization, as desired, such as one or more scavengers, promoters, modifiers, chain transfer agents (such as diethyl zinc), reducing agents, oxidizing agents, hydrogen, aluminum alkyls, or silanes.

Useful chain transfer agents are typically alkylalumoxanes, a compound represented by the formula AIR₃, ZnR₂ (where each R is, independently, a C₁-C₈ aliphatic radical, such as methyl, ethyl, propyl, butyl, phenyl, hexyl octyl or an isomer thereof) or a combination thereof, such as diethyl zinc, methylalumoxane, trimethylaluminum, triisobutylaluminum, trioctylaluminum, or a combination thereof.

### Gas Phase Polymerization

Generally, in a fluidized gas bed process used for producing polymers, a gaseous stream containing one or more monomers is continuously cycled through a fluidized bed in the presence of a catalyst under reactive conditions. The gaseous stream is withdrawn from the fluidized bed and recycled back into the reactor. Simultaneously, polymer product is withdrawn from the reactor and fresh monomer is added to replace the polymerized monomer. (See, for example, U.S. Pat. Nos. 4,543,399; 4,588,790; 5,028,670; 5,317,036; 5,352,749; 5,405,922; 5,436,304; 5,453,471; 5,462,999; 5,616,661; and 5,668,228; all of which are fully incorporated herein by reference.)

### Slurry Phase Polymerization

A slurry polymerization process generally operates between 1 to about 50 atmosphere pressure range (15 psi to 735 psi, 103 kPa to 5,068 kPa) or even greater and temperatures in the range of 0° C. to about 120° C. In a slurry polymerization, a suspension of solid, particulate polymer is formed in a liquid polymerization diluent medium to which monomer and comonomers, along with catalysts, are added. The suspension including diluent is intermittently or continuously removed from the reactor where the volatile components are separated from the polymer and recycled, optionally after a distillation, to the reactor. The liquid diluent used in the polymerization medium is typically an alkane having from 3 to 7 carbon atoms, such as a branched alkane. The medium employed should be liquid under the conditions of polymerization and relatively inert. When a propane medium is used, the process must be operated above the reaction diluent critical temperature and pressure. For example, a hexane or an isobutane medium is employed.

In at least one embodiment, a polymerization process is a particle form polymerization, or a slurry process, where the temperature is kept below the temperature at which the polymer goes into solution. Such technique is well known in the art, and described in for instance U.S. Pat. No. 3,248,179 which is fully incorporated herein by reference. The temperature in the particle form process can be from about 85° C. to about 110° C. Two example polymerization methods for the slurry process are those using a loop reactor and those utilizing a plurality of stirred reactors in series, parallel, or combinations thereof. Non-limiting examples of slurry processes include continuous loop or stirred tank processes. Also, other examples of slurry processes are described in U.S. Pat. No. 4,613,484, which is herein fully incorporated by reference.

In another embodiment, the slurry process is carried out continuously in a loop reactor. The catalyst, as a slurry in isohexane or as a dry free flowing powder, is injected regularly to the reactor loop, which is itself filled with circulating slurry of growing polymer particles in a diluent of isohexane containing monomer and optional comonomer. Hydrogen, optionally, may be added as a molecular weight control. (In one embodiment hydrogen is added from 50 ppm to 500 ppm, such as from 100 ppm to 400 ppm, such as 150 ppm to 300 ppm.)

The reactor may be maintained at a pressure of 2,000 kPa to 5,000 kPa, such as from 3,620 kPa to 4,309 kPa, and at a temperature of from about 60° C. to about 120° C. depending on the desired polymer melting characteristics. Reaction heat is removed through the loop wall since much of the reactor is in the form of a double-jacketed pipe. The slurry is allowed to exit the reactor at regular intervals or continuously to a heated low pressure flash vessel, rotary dryer and a nitrogen purge column in sequence for removal of the isohexane diluent and all unreacted monomer and comonomer. The resulting hydrocarbon free powder is then compounded for use in various applications.

Other additives may also be used in the polymerization, as desired, such as one or more scavengers, promoters, modifiers, chain transfer agents (such as diethyl zinc), reducing agents, oxidizing agents, hydrogen, aluminum alkyls, or silanes.

Useful chain transfer agents are typically alkylalumoxanes, a compound represented by the formula AIR₃, ZnR₂ (where each R is, independently, a C₁-C₈ hydrocarbyl, such as methyl, ethyl, propyl, butyl, pentyl, hexyl octyl or an isomer thereof). Examples can include diethyl zinc, methylalumoxane, trimethylaluminum, triisobutylaluminum, trioctylaluminum, or a combination thereof.

### Solution Polymerization

A solution polymerization is a polymerization process in which the polymer is dissolved in a liquid polymerization medium, such as an inert solvent or monomer(s) or their blends. A solution polymerization is typically homogeneous. A homogeneous polymerization is one where the polymer product is dissolved in the polymerization medium. Such systems are typically not turbid as described in Oliveira, J. Vladimir et al. (2000) “High-Pressure Phase Equilibria for Polypropylene-Hydrocarbon Systems,” Ind. Eng. Chem. Res., v 29, pp. 4627-4633. Generally solution polymerization involves polymerization in a continuous reactor in which the polymer formed and the starting monomer and catalyst materials supplied, are agitated to reduce or avoid concentration gradients and in which the monomer acts as a diluent or solvent or in which a hydrocarbon is used as a diluent or solvent. Suitable processes typically operate at temperatures from about 0° C. to about 250° C., such as about 10° C. to about 150° C., such as about 40° C. to about 140° C., such as about 50° C. to about 120° C., and at pressures of about 0.1 MPa or more, such as 2 MPa or more. The upper pressure limit is not critically constrained but typically can be about 200 MPa or less, such as 120 MPa or less. Temperature control in the reactor can generally be obtained by balancing the heat of polymerization and with reactor cooling by reactor jackets or cooling coils to cool the contents of the reactor, auto refrigeration, pre-chilled feeds, vaporization of liquid medium (diluent, monomers or solvent) or combinations of all three. Adiabatic reactors with pre-chilled feeds can also be used. The purity, type, and amount of solvent can be optimized for the maximum catalyst productivity for a particular type of polymerization. The solvent can be also introduced as a catalyst carrier. The solvent can be introduced as a gas phase or as a liquid phase depending on the pressure and temperature. Advantageously, the solvent can be kept in the liquid phase and introduced as a liquid. Solvent can be introduced in the feed to the polymerization reactors.

### Polyolefin Products

The present disclosure also provides compositions of matter which can be produced by the methods described herein.

In at least one embodiment, a polyolefin is a propylene homopolymer, an ethylene homopolymer or an ethylene copolymer, such as propylene-ethylene and/or ethylene-alphaolefin (such as C₄ to C₂₀) copolymer (such as an ethylene-hexene copolymer or an ethylene-octene copolymer). A polyolefin can have an Mw/Mn of greater than 1 to 4 (such as greater than 1 to 3).

In at least one embodiment, a polyolefin is a homopolymer of ethylene or propylene or a copolymer of ethylene such as a copolymer of ethylene having from 0.1 to 25 wt % (such as from 0.5 to 20 wt %, such as from 1 to 15 wt %, such as from 5 to 17 wt %) of ethylene with the remainder balance being one or more C₃ to C₂₀ olefin comonomers (such as C₃ to C₁₂ alpha-olefin, such as propylene, butene, hexene, octene, decene, dodecene, such as propylene, butene, hexene, octene). A polyolefin can be a copolymer of propylene such as a copolymer of propylene having from 0.1 to 25 wt % (such as from 0.5 to 20 wt %, such as from 1 to 15 wt %, such as from 3 to 10 wt %) of propylene and from 99.9 to 75 wt % of one or more of C₂ or C₄ to C₂₀ olefin comonomer (such as ethylene or C₄ to C₁₂ alpha-olefin, such as butene, hexene, octene, decene, dodecene, such as ethylene, butene, hexene, octene).

In at least one embodiment, a polyolefin, such as a polypropylene (e.g., iPP) or an ethylene-octene copolymer, has an Mw from 40,000 to 1,500,000 g/mol, such as from 70,000 to 1,000,000 g/mol, such as from 90,000 to 500,000 g/mol, such as from 90,000 to 250,000 g/mol, such as from 90,000 to 200,000 g/mol, such as from 90,000 to 110,000 g/mol.

In at least one embodiment, a polyolefin, such as a polypropylene (e.g., iPP) or an ethylene-octene copolymer, has an Mn from 5,000 to 1,000,000 g/mol, such as from 20,000 to 160,000 g/mol, such as from 30,000 to 70,000 g/mol, such as from 40,000 to 70,000 g/mol. In at least one embodiment, a polyolefin, such as a polypropylene (e.g., iPP) or an ethylene-octene copolymer, has an Mw/Mn value from 1 to 10, such as from 1.5 to 9, such as from 2 to 7, such as from 2 to 4, such as from 2.5 to 3, for example about 2.

In at least one embodiment, a polyolefin, such as a polypropylene (e.g., iPP) or an ethylene-octene copolymer, has a melt temperature (Tm) of from 100° C. to 150° C., such as 110° C. to 140° C., such as 120° C. to 135° C., such as 130° C. to 135° C.

In at least one embodiment, a polymer of the present disclosure has a g′ᵥᵢₛ of greater than 0.9, such as greater than 0.92, such as greater than 0.95.

In at least one embodiment, the polymer is an ethylene copolymer, and the comonomer is octene, at a comonomer content of from 1 wt % to 18 wt % octene, such as from 5 wt % to 15 wt %, such as from 8 wt % to 13 wt %, such as from 9 wt % to 12 wt %.

In at least one embodiment, the polymer produced herein has a unimodal or multimodal molecular weight distribution as determined by Gel Permeation Chromatography (GPC). By “unimodal” is meant that the GPC trace has one peak or inflection point. By “multimodal” is meant that the GPC trace has at least two peaks or inflection points. An inflection point is that point where the second derivative of the curve changes in sign (e.g., from negative to positive or vice versus).

In at least one embodiment, the polymer produced herein has a composition distribution breadth index (CDBI) of 50% or more, such as 60% or more, such as 70% or more. CDBI is a measure of the composition distribution of monomer within the polymer chains and is measured by the procedure described in PCT publication WO 1993/003093, published Feb. 18, 1993, specifically columns 7 and 8 as well as in Wild, L. et al (1982) “Determination of Branching Distributions in Polyethylene and Ethylene Copolymers,” J. Poly. Sci., Poly. Phys. Ed., v. 20, p. 441-455 and U.S. Pat. No. 5,008,204, including that fractions having a weight average molecular weight (Mw) below 15,000 are ignored when determining CDBI.

Copolymer of the present disclosure can have a reversed comonomer index. The reversed-co-monomer index (RCI,m) is computed from x2 (mol % co-monomer C₃, C₄, C₆, C₈, etc.), as a function of molecular weight, where x2 is obtained from the following expression in which n is the number of carbon atoms in the comonomer (3 for C₃, 4 for C₄, 6 for C₆, etc.):

$$x 2 = - \frac{200 w 2}{- 100 n - 2 w 2 + nw 2}$$

Then the molecular-weight distribution, W(z) where z=log₁₀ M, is modified to W′(z) by setting to 0 the points in W that are less than 5% of the maximum of W; this is to effectively remove points for which the S/N in the composition signal is low. Also, points of W′ for molecular weights below 2000 gm/mole are set to 0. Then W′ is renormalized so that

$$1 = {\int }_{- ∞}^{∞} {W}^{′} dz$$

and a modified weight-average molecular weight (M_{w}′) is calculated over the effectively reduced range of molecular weights as follows:

$${M}_{W}^{′} = {\int }_{- ∞}^{∞} {10}^{z} * {W}^{′} dz .$$

The RCI,m is then computed as:

$$RCI , m = {\int }_{- ∞}^{∞} x 2 ( {10}^{z} - {M}_{W}^{′} ) {W}^{′} dz$$

A reversed-co-monomer index (RCI,w) is also defined on the basis of the weight fraction co-monomer signal (w2/100) and is computed as follows:

$$RCI , w = {\int }_{- ∞}^{∞} \frac{w 2}{100} ( {10}^{z} - {M}_{W}^{′} ) {W}^{′} dz .$$

Note that in the above definite integrals the limits of integration are the widest possible for the sake of generality; however, in reality the function is only integrated over a finite range for which data is acquired, considering the function in the rest of the non-acquired range to be 0. Also, by the manner in which W′ is obtained, it is possible that W′ is a discontinuous function, and the above integrations need to be done piecewise.

Three co-monomer distribution ratios are also defined on the basis of the % weight (w2) comonomer signal, denoted as CDR-1,w, CDR-2,w, and CDR-3,w, as follows:

$$CDR \text{-} 1 , w = \frac{w 2 ( Mz )}{w 2 ( Mw )}$$

$$CDR \text{-} 2 , w = \frac{w 2 ( Mz )}{w 2 ( \frac{Mw + Mn}{2} )}$$

$$CDR \text{-} 3 , w = \frac{w 2 ( \frac{Mz + Mw}{2} )}{w 2 ( \frac{Mw + Mn}{2} )}$$

where w2(Mw) is the % weight co-monomer signal corresponding to a molecular weight of Mw, w2(Mz) is the % weight co-monomer signal corresponding to a molecular weight of Mz, w2[(Mw+Mn)/2)] is the % weight co-monomer signal corresponding to a molecular weight of (Mw+Mn)/2, and w2[(Mz+Mw)/2] is the % weight co-monomer signal corresponding to a molecular weight of Mz+Mw/2, where Mw is the weight-average molecular weight, Mn is the number-average molecular weight, and Mz is the z-average molecular weight.

Accordingly, the co-monomer distribution ratios can be also defined utilizing the % mole co-monomer signal, CDR-1,m, CDR-2,m, CDR-3,m, as:

$$CDR \text{-} 1 , m = \frac{x 2 ( Mz )}{x 2 ( Mw )}$$

$$CDR \text{-} 2 , m = \frac{x 2 ( Mz )}{x 2 ( \frac{Mw + Mn}{2} )}$$

$$CDR \text{-} 3 , m = \frac{x 2 ( \frac{Mz + Mw}{2} )}{x 2 ( \frac{Mw + Mn}{2} )}$$

where x2(Mw) is the % mole co-monomer signal corresponding to a molecular weight of Mw, x2(Mz) is the % mole co-monomer signal corresponding to a molecular weight of Mz, x2[(Mw+Mn)/2)] is the % mole co-monomer signal corresponding to a molecular weight of (Mw+Mn)/2, and x2[(Mz+Mw)/2] is the % mole co-monomer signal corresponding to a molecular weight of Mz+Mw/2, where Mw is the weight-average molecular weight, Mn is the number-average molecular weight, and Mz is the z-average molecular weight.

In at least one embodiment of the present disclosure, the polymer produced by the processes described herein includes ethylene and one or more comonomers and the polymer has: 1) an RCI,m of 30 or more (alternatively from 30 to 250).

Molecular Weight, Comonomer Composition and Long Chain Branching Determination by Polymer Char GPC-IR Hyphenated with Multiple Detectors

The distribution and the moments of molecular weight (Mw, Mn, Mw/Mn, etc.), the comonomer content (C2, C3, C6, etc.) and the long chain branching (g′) are determined by using a high temperature Gel Permeation Chromatography (Polymer Char GPC-IR) equipped with a multiple-channel band-filter based Infrared detector IR5, an 18-angle light scattering detector and a viscometer. Three Agilent PLgel 10 μm Mixed-B LS columns are used to provide polymer separation. Aldrich reagent grade 1,2,4-trichlorobenzene (TCB) with 300 ppm antioxidant butylated hydroxytoluene (BHT) is used as the mobile phase. The TCB mixture is filtered through a 0.1 μm Teflon filter and degassed with an online degasser before entering the GPC instrument. The nominal flow rate is 1.0 mL/min and the nominal injection volume is 200 μL. The whole system including transfer lines, columns, detectors are contained in an oven maintained at 145° C. Given amount of polymer sample is weighed and sealed in a standard vial with 80 μL flow marker (Heptane) added to it. After loading the vial in the autosampler, polymer is automatically dissolved in the instrument with 8 mL added TCB solvent. The polymer is dissolved at 160° C. with continuous shaking for about 1 hour for most PE samples or 2 hour for PP samples. The TCB densities used in concentration calculation are 1.463 g/ml at room temperature and 1.284 g/ml at 145° C. The sample solution concentration is from 0.2 to 2.0 mg/ml, with lower concentrations being used for higher molecular weight samples.

The concentration (c), at each point in the chromatogram is calculated from the baseline-subtracted IR5 broadband signal intensity (I), using the following equation:

c=βI

where β is the mass constant determined with PE or PP standards. The mass recovery is calculated from the ratio of the integrated area of the concentration chromatography over elution volume and the injection mass which is equal to the pre-determined concentration multiplied by injection loop volume.

The conventional molecular weight (IR MW) is determined by combining universal calibration relationship with the column calibration which is performed with a series of monodispersed polystyrene (PS) standards ranging from 700 to 10M. The MW at each elution volume is calculated with following equation.

$$log M = \frac{log ( {K}_{PS} / K )}{a + 1} + \frac{{a}_{PS} + 1}{a + 1} {log M}_{PS}$$

where the variables with subscript “PS” stands for polystyrene while those without a subscript are for the test samples. In this method, a_{PS}=0.67 and K_{PS}=0.000175 while a and K are calculated from a series of empirical formula established in ExxonMobil and published in literature (Sun, T. et al. (2001) “Effect of Short Chain Branching on the Coil Dimensions of Polyolefins in Dilute Solution,” Macromolecules, v. 34(19), pp. 6812-6820). Specifically, a/K=0.695/0.000579 for PE and 0.705/0.0002288 for PP.

The comonomer composition is determined by the ratio of the IR5 detector intensity corresponding to CH₂ and CH₃ channel calibrated with a series of PE and PP homo/copolymer standards whose nominal value are predetermined by NMR or FTIR such as EMCC commercial grades about LLDPE, Vistamaxx, ICP, etc.

The LS detector is the 18-angle Wyatt Technology High Temperature DAWN HELEOSII. The LS molecular weight (M) at each point in the chromatogram is determined by analyzing the LS output using the Zimm model for static light scattering (M. B. Huglin (1971) Light Scattering from Polymer Solutions, Academic Press):

$$\frac{{K}_{o} c}{Δ R ( θ )} = \frac{1}{MP ( θ )} + 2 {A}_{2} c$$

Here, ΔR(θ) is the measured excess Rayleigh scattering intensity at scattering angle θ, c is the polymer concentration determined from the IR5 analysis, A2 is the second virial coefficient. P(θ) is the form factor for a monodisperse random coil, and Kₒ is the optical constant for the system:

$${K}_{o} = \frac{4 {π}^{2} {{n}^{2} ( dn / dc )}^{2}}{{λ}^{4} {N}_{A}}$$

where N_{A} is Avogadro's number, and (dn/dc) is the refractive index increment for the system. The refractive index, n=1.500 for TCB at 145° C. and X=665 nm.

A high temperature Agilent (or Viscotek Corporation) viscometer, which has four capillaries arranged in a Wheatstone bridge configuration with two pressure transducers, is used to determine specific viscosity. One transducer measures the total pressure drop across the detector, and the other, positioned between the two sides of the bridge, measures a differential pressure. The specific viscosity, ηₛ, for the solution flowing through the viscometer is calculated from their outputs. The intrinsic viscosity, [η], at each point in the chromatogram is calculated from the following equation:

[η]=η_{S}/c

where c is concentration and was determined from the IR5 broadband channel output. The viscosity MW at each point is calculated from the below equation:

M=K_{PS}M^{α}^{_{PS}}⁺¹+/[η]

The branching index (g′ᵥᵢₛ) is calculated using the output of the GPC-IR5-LS-VIS method as follows. The average intrinsic viscosity, [η]_{avg}, of the sample is calculated by:

$${[ η ]}_{avg} = \frac{\sum {{c}_{i} [ η ]}_{i}}{\sum {c}_{i}}$$

where the summations are over the chromatographic slices, i, between the integration limits. The branching index g′ᵥᵢₛ is defined as:

$${g}_{vis}^{′} = \frac{{[ η ]}_{avg}}{{kM}_{v}^{α}}$$

M_{V} is the viscosity-average molecular weight based on molecular weights determined by LS analysis. The K/a are for the reference linear polymer which is usually PE with certain amount of short chain branching.

All the concentration is expressed in g/cm³, molecular weight is expressed in g/mole, and intrinsic viscosity is expressed in dL/g unless otherwise noted.

All molecular weights are reported in g/mol unless otherwise noted.

Differential Scanning Calorimetry (DSC-Procedure-2). Melting Temperature, Tm, is measured by differential scanning calorimetry (“DSC”) using a DSCQ200 unit. The sample is first equilibrated at 25° C. and subsequently heated to 220° C. using a heating rate of 10° C./min (first heat). The sample is held at 220° C. for 3 min. The sample is subsequently cooled down to −100° C. with a constant cooling rate of 10° C./min (first cool). The sample is equilibrated at −100° C. before being heated to 220° C. at a constant heating rate of 10° C./min (second heat). The exothermic peak of crystallization (first cool) is analyzed using the TA Universal Analysis software and the corresponding to 10° C./min cooling rate is determined. The endothermic peak of melting (second heat) is also analyzed using the TA Universal Analysis software and the peak melting temperature (Tm) corresponding to 10° C./min heating rate is determined. In the event of conflict between the DSC Procedure-1 and DSC procedure-2, DSC procedure-2 is used.

### Blends

In another embodiment, the polymer (such as the polyethylene or polypropylene) produced herein is combined with one or more additional polymers prior to being formed into a film, molded part or other article. Other useful polymers include polyethylene, isotactic polypropylene, highly isotactic polypropylene, syndiotactic polypropylene, random copolymer of propylene and ethylene, and/or butene, and/or hexene, polybutene, ethylene vinyl acetate, low density polyethylene (LDPE), linear low density polyethylene (LLDPE), high density polyethylene (HDPE), ethylene vinyl acetate, ethylene methyl acrylate, copolymers of acrylic acid, polymethylmethacrylate or any other polymers polymerizable by a high-pressure free radical process, polyvinylchloride, polybutene-1, isotactic polybutene, ABS resins, ethylene-propylene rubber (EPR), vulcanized EPR, EPDM, block copolymer, styrenic block copolymers, polyamides, polycarbonates, PET resins, cross linked polyethylene, copolymers of ethylene and vinyl alcohol (EVOH), polymers of aromatic monomers such as polystyrene, poly-1 esters, polyacetal, polyvinylidine fluoride, polyethylene glycols, and/or polyisobutylene.

In at least one embodiment, the polymer (such as polyethylene or polypropylene) is present in the above blends, at from 10 to 99 wt %, based upon the weight of the polymers in the blend, such as 20 to 95 wt %, such as at least 30 to 90 wt %, such as at least 40 to 90 wt %, such as at least 50 to 90 wt %, such as at least 60 to 90 wt %, such as at least 70 to 90 wt %.

The blends described above may be produced by mixing the polymers of the present disclosure with one or more polymers (as described above), by connecting reactors together in series to make reactor blends or by using more than one catalyst in the same reactor to produce multiple species of polymer. The polymers can be mixed together prior to being put into the extruder or may be mixed in an extruder.

The blends may be formed using conventional equipment and methods, such as by dry blending the individual components and subsequently melt mixing in a mixer, or by mixing the components together directly in a mixer, such as, for example, a Banbury mixer, a Haake mixer, a Brabender internal mixer, or a single or twin-screw extruder, which may include a compounding extruder and a side-arm extruder used directly downstream of a polymerization process, which may include blending powders or pellets of the resins at the hopper of the film extruder. Additionally, additives may be included in the blend, in one or more components of the blend, and/or in a product formed from the blend, such as a film, as desired. Such additives are well known in the art, and can include, for example: fillers; antioxidants (e.g., hindered phenolics such as IRGANOX™ 1010 or IRGANOX™ 1076 available from Ciba-Geigy); phosphites (e.g., IRGAFOS™ 168 available from Ciba-Geigy); anti-cling additives; tackifiers, such as polybutenes, terpene resins, aliphatic and aromatic hydrocarbon resins, alkali metal and glycerol stearates, and hydrogenated rosins; UV stabilizers; heat stabilizers; anti-blocking agents; release agents; anti-static agents; pigments; colorants; dyes; waxes; silica; fillers; and talc.

### Films

One or more of the foregoing polymers, such as the foregoing polyethylenes, polypropylenes, or blends thereof, may be used in a variety of end-use applications. Such applications include, for example, mono- or multi-layer blown, extruded, and/or shrink films. These films may be formed by any number of well-known extrusion or coextrusion techniques, such as a blown bubble film processing technique, wherein the composition can be extruded in a molten state through an annular die and then expanded to form a uni-axial or biaxial orientation melt prior to being cooled to form a tubular, blown film, which can then be axially slit and unfolded to form a flat film. Films may be subsequently unoriented, uniaxially oriented, or biaxially oriented to the same or different extents. One or more of the layers of the film may be oriented in the transverse and/or longitudinal directions to the same or different extents. The uniaxially orientation can be accomplished using typical cold drawing or hot drawing methods. Biaxial orientation can be accomplished using tenter frame equipment or a double bubble processes and may occur before or after the individual layers are brought together. For example, a polyethylene layer can be extrusion coated or laminated onto an oriented polypropylene layer or the polyethylene and polypropylene can be coextruded together into a film then oriented. Likewise, oriented polypropylene could be laminated to oriented polyethylene or oriented polyethylene could be coated onto polypropylene then optionally the combination could be oriented even further. Typically the films are oriented in the Machine Direction (MD) at a ratio of up to 15, such as between 5 and 7, and in the Transverse Direction (TD) at a ratio of up to 15, such as 7 to 9. However, in at least one embodiment the film is oriented to the same extent in both the MD and TD directions.

The films may vary in thickness depending on the intended application; however, films of a thickness from 1 μm to 50 μm are usually suitable. Films intended for packaging are usually from 10 μm to 50 μm thick. The thickness of the sealing layer is typically 0.2 μm to 50 μm. There may be a sealing layer on both the inner and outer surfaces of the film or the sealing layer may be present on only the inner or the outer surface.

This invention further relates to:

1. A compound represented by Formula (AI):

[R¹R²R³EH]^{d+}[M^{k+}Qₙ]^{d-} (AI)

wherein:
E is nitrogen or phosphorous;
d is 1, 2 or 3; k is 1, 2, or 3; n is 1, 2, 3, 4, 5, or 6; n−k=d;
R¹ is electron deficient aromatic group;
each R², and R³ is independently C₁-C₄₀ linear alkyl or C₅-C₅₀-aryl, wherein each of R² and R³ is independently unsubstituted or substituted with at least one of halide, C₁-C₅₀ alkyl, C₅-C₅₀ aryl, C₆-C₃₅ arylalkyl, or C₆-C₃₅ alkylaryl, wherein R¹, R², and R³ together comprise 15 or more carbon atoms; M is an element selected from group 13 of the Periodic Table of the Elements; and each Q is independently a hydride, bridged or unbridged dialkylamido, halide, alkoxide, aryloxide, hydrocarbyl, substituted hydrocarbyl, halocarbyl, substituted halocarbyl, or halosubstituted-hydrocarbyl radical, provided that when each Q is perfluorophenyl, then R¹ is not methyl, and R² and R³ are not C₁₈ alkyl.
2. The compound of paragraph 1 wherein the compound represented by Formula (I):

[R¹R²R³EH]⁺[BR⁴R⁵R⁶R⁷]⁻ (I)

wherein:
E is nitrogen or phosphorous, preferable nitrogen;
R¹ is electron deficient aromatic group, each of R² and R³ is independently C₁-C₄₀ alkyl, C₅-C₂₂-aryl, wherein each of R² and R³ is independently unsubstituted or substituted with at least one of halide, C₁-C₁₀ alkyl, C₅-C₁₅ aryl, C₆-C₂₅ arylalkyl, and C₆-C₂₅ alkylaryl, wherein R¹, R², and R³ together comprise 15 or more carbon atoms;
each of R⁴, R⁵, R⁶, and R⁷ is aryl (such as phenyl or naphthyl), wherein at least one of R⁴, R⁵, R⁶, and R⁷ is substituted with from one or more fluorine atoms.
3. The compound of paragraph 1 or 2, wherein at least one of R⁴, R⁵, R⁶, and R⁷ is naphthyl substituted with seven fluorine atoms.
4. The compound of any of paragraphs 1 to 3, wherein each of R⁴, R⁵, R⁶, and R⁷ is phenyl substituted with from one to five fluorine atoms.
5. The compound of paragraph 1 or 2, wherein each of R⁴, R⁵, R⁶, and R⁷ is naphthyl substituted with seven fluorine atoms.
6. The compound of any of paragraphs 1 to 5, wherein R¹, R², and R³ together comprise 17 or more carbon atoms.
7. The compound of any of paragraphs 1 to 6, wherein R¹, R², and R³ together comprise 20 or more carbon atoms.
8. The compound of any of paragraphs 1 to 7, wherein R¹, R², and R³ together comprise 25 or more carbon atoms.
9. The compound of any of paragraphs 1 to 8, wherein R¹, R², and R³ together comprise 35 or more carbon atoms.
10. The compound of any of paragraphs 1 to 9, wherein R¹ is a phenyl group that is substituted with one, two, three, four or five halogen and/or haloalkyl groups and each of R² and R³ is independently C₁-C₄₀ linear alkyl, or C₅-C₂₂-aryl, wherein each of R² and R³ is independently unsubstituted or substituted with at least one C₁-C₁₀ alkyl.
11. The compound of any of paragraphs 1 to 10, wherein R¹ is a phenyl group, that is substituted with one, two, three, four or five groups selected from fluoro, chloro, bromo, iodo, and trifluoromethyl and each of R² and R³ is independently selected from methyl, ethyl, n-propyl, n-butyl, n-pentyl, n-hexyl, n-heptyl, n-octyl, n-nonyl, n-decyl, n-undecyl, n-dodecyl, n-tridecyl, n-butadecyl, n-pentadecyl, n-hexadecyl, n-heptadecyl, n-octadecyl, n-nonadecyl, cyclohexylmethyl, and n-icosyl.
12. The compound of any of paragraphs 1 to 11, wherein R¹ is selected from 4-fluorophenyl, 4-(trifluoromethyl)phenyl, 3-fluorophenyl, 3-(trifluoromethyl)phenyl, and 3-chlorophenyl.
13. The compound of paragraph 1, wherein R¹ is F₃C-phenyl or F-phenyl, R² is C₁₀-C₄₀ alkyl, and R³ is C₁₀-C₄₀ alkyl.
14. The compound of paragraph 1, wherein the compound represented by formula (I) comprises a cation, [R¹R²R³EH]+, selected from the group consisting of:
N,N-didodecyl-2,3,4,5,6-pentafluorobenzenaminium,
N,N-didodecyl-3,5-difluorobenzenaminium,
N,N-didodecyl-3,5-bis(trifluoromethyl)benzenaminium,
N,N-bis(cyclohexylmethyl)-2,3,4,5,6-pentafluorobenzenaminium,
N,N-bis(cyclohexylmethyl)-3,5-bis(trifluoromethyl)benzenaminium,
N,N-bis(cyclohexylmethyl)-4-(trifluoromethyl)benzenaminium,
N,N-bis(cyclohexylmethyl)-4-fluorobenzenaminium, and
N,N-didodecyl-4-(trifluoromethyl)anilium.
   15. The compound of paragraph 1, wherein the compound represented by formula (I) comprises a cation, [R¹R²R³EH]+, selected from the group consisting of N,N-bis(cyclohexylmethyl)-4-(trifluoromethyl)benzenaminium, N,N-bis(cyclohexylmethyl)-4-fluorobenzenaminium, and N,N-didodecyl-4-(trifluoromethyl)anilium.
   16. A catalyst system comprising a catalyst and the activator compound of any of paragraphs 1 to 15.
   17. The catalyst system of paragraph 16, further comprising a support material.
   18. The catalyst system of paragraph 17, wherein the support material is selected from Al₂O₃, ZrO₂, SiO₂, SiO₂/Al₂O₃, SiO₂/TiO₂, silica clay, silicon oxide/clay, or mixtures thereof.
   19. The catalyst system of any of paragraphs 16 to 18, wherein the catalyst is represented by formula (II) or formula (III):

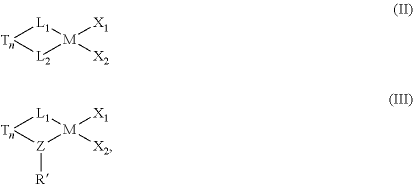

wherein in each of formula (II) and formula (III):
M is the metal center, and is a Group 4 metal;

### n is O or 1;

T is an optional bridging group selected from dialkylsilyl, diarylsilyl, dialkylmethyl, ethylenyl or hydrocarbylethylenyl wherein one, two, three or four of the hydrogen atoms in ethylenyl are substituted by hydrocarbyl;
Z is nitrogen, oxygen or phosphorus;
R′ is a C₁-C₄₀ alkyl or substituted akyl group, preferably a linear C₁-C₄₀ alkyl or substituted alkyl group;
X₁ and X₂ are, independently, hydrogen, halogen, hydride radicals, hydrocarbyl radicals, substituted hydrocarbyl radicals, halocarbyl radicals, substituted halocarbyl radicals, silylcarbyl radicals, substituted silylcarbyl radicals, germylcarbyl radicals, or substituted germylcarbyl radicals; or both X₁ and X₂ are joined and bound to the metal atom to form a metallacycle ring containing from about 3 to about 20 carbon atoms; or both together can be an olefin, diolefin or aryne ligand.
20. The catalyst system of paragraph 19, wherein the catalyst is C₂ symmetrical.
21. The catalyst system of any of paragraphs 16 to 19, wherein the catalyst is rac-dimethylsilyl-bis(indenyl)hafnium dimethyl.
22. The catalyst system of any of paragraphs 16 to 19, wherein the catalyst is one or more of:
bis(1-methyl, 3-n-butyl cyclopentadienyl) M(R)₂;
dimethylsilyl bis(indenyl)M(R)₂;
bis(indenyl)M(R)₂;
dimethylsilyl bis(tetrahydroindenyl)M(R)₂;
bis(n-propylcyclopentadienyl)M(R)₂;
dimethylsilyl (tetramethylcyclopentadienyl)(cyclododecylamido)M(R)₂;
dimethylsilyl (tetramethylcyclopentadienyl)(cyclododecylamido)M(R)₂;
dimethylsilyl (tetramethylcyclopentadienyl)(t-butylamido)M(R)₂;
dimethylsilyl (tetramethylcyclopentadienyl)(t-butylamido)M(R)₂;
μ-(CH₃)₂Si(cyclopentadienyl)(1-adamantylamido)M(R)₂;
μ-(CH₃)₂Si(3-tertbutylcyclopentadienyl)(1-adamantylamido)M(R)₂;
μ-(CH₃)₂(tetramethylcyclopentadienyl)(1-adamantylamido)M(R)₂;
μ-(CH₃)₂Si(tetramethylcyclopentadienyl)(1-adamantylamido)M(R)₂;
μ-(CH₃)₂C(tetramethylcyclopentadienyl)(1-adamantylamido)M(R)₂;
μ-(CH₃)₂Si(tetramethylcyclopentadienyl)(1-tertbutylamido)M(R)₂;
μ-(CH₃)₂Si(fluorenyl)(1-tertbutylamido)M(R)₂;
μ-(CH₃)₂Si(tetramethylcyclopentadienyl)(1-cyclododecylamido)M(R)₂;
μ-(C₆H5)₂C(tetramethylcyclopentadienyl)(1-cyclododecylamido)M(R)₂;
μ-(CH₃)₂Si(15-2,6,6-trimethyl-1,5,6,7-tetrahydro-s-indacen-1-yl)(tertbutylamido)M(R)₂;
   where M is selected from Ti, Zr, and Hf; and R is selected from halogen or C₁ to C₅ alkyl.
   23. The catalyst system of paragraph 16, 17 or 18, wherein the catalyst is represented by the catalyst compound (BI) (BII), (BIII), (CI), (CII), (CIII), (IV), (VII), (VIII), (IX), (X), (XI), (XII), (XIII), (XIV), or (XV), as described herein.
   24. A method of polymerizing olefins to produce at least one polyolefin, the method comprising: contacting at least one olefin with the catalyst system of any of paragraphs 16 to 23; and obtaining a polyolefin.
   25. The method of paragraph 24, wherein the at least one olefin is propylene and the polyolefin is isotactic polypropylene.
   26. A method of polymerizing olefins to produce at least one polyolefin, the method comprising: contacting two or more different olefins (preferably ethylene and propylene) with the catalyst system of any of paragraphs 16 to 25; and obtaining a polyolefin.
   27. The method of paragraph 26, wherein the two or more olefins further comprise a diene.
   28. The method of any of paragraphs 26-27, wherein the polyolefin has an Mw of from about 50,000 to about 300,000 g/mol and a melt temperature of from about 120° C. to about 140° C.
   29. The method of paragraph 28, wherein the polyolefin has an Mw of from about 100,000 to about 300,000 g/mol and a melt temperature of from about 125° C. to about 135° C.
   30. The method of any of paragraphs 24 to 29, wherein the method is performed in gas phase or slurry phase.
   31. The method of any of paragraphs 24 to 29, wherein the method is performed in solution phase.
   32. A solution comprising the compound of any of paragraphs 1-15 or the catalyst system of any of paragraphs 16-23 and an aliphatic solvent.
   33. The solution of paragraph 32 where aromatic solvents, such as toluene, are absent.
   34. A composition comprising the catalyst system of any of paragraphs 17 to 23 and an aliphatic solvent, where aromatic solvents, such as toluene, are absent.

### EXPERIMENTAL

Lithium tetrakis(pentafluorophenyl)borate etherate (Li—BF20) was purchased from Boulder Scientific. N,N-Dimethylanilinium tetrakis(pentafluorophenyl)borate (DMAH-BF20) was purchased from Albemarle Corporation, Baton Rouge, La. Sodium tetrakis(heptafluoronaphthalen-2-yl)borate (Na—BF28) and N,N-dimethylanilinium tetrakis(heptafluoronaphthalen-2-yl)borate (DMAH-BF28) were purchased from Grace Davison. Di(hydrogenated tallow)methylamine (M2HT) was purchased from Akzo Nobel. Didocecylmethylamine, tridodecylamine, p-toluidine, Celite, silica gel, ethereal HCl (2M) were purchased from Sigma-Aldrich. N-methyldioctadecylamine was provided by AkzoNobel.

¹H NMR for Compound Characterization:

Chemical structures are determined by ¹H NMR. ¹H NMR data are collected at room temperature (e.g., 23° C.) in a 5 mm probe. The ¹H NMR measurements were recorded on a 400 MHz or 500 MHz Bruker spectrometer with chemical shifts referenced to residual solvent peaks (CDCl₃: 7.27 ppm for ¹H, 77.23 ppm for ¹³C).

All anhydrous solvents were purchased from Sigma-Aldrich. Deuterated solvents were purchased from Cambridge Isotope Laboratories.

### Examples

Borate anions and ammonium cations used as activator components are shown in Scheme 1.

Scheme 1: Borate Anions and Ammonium Cations Used as Activator Components.

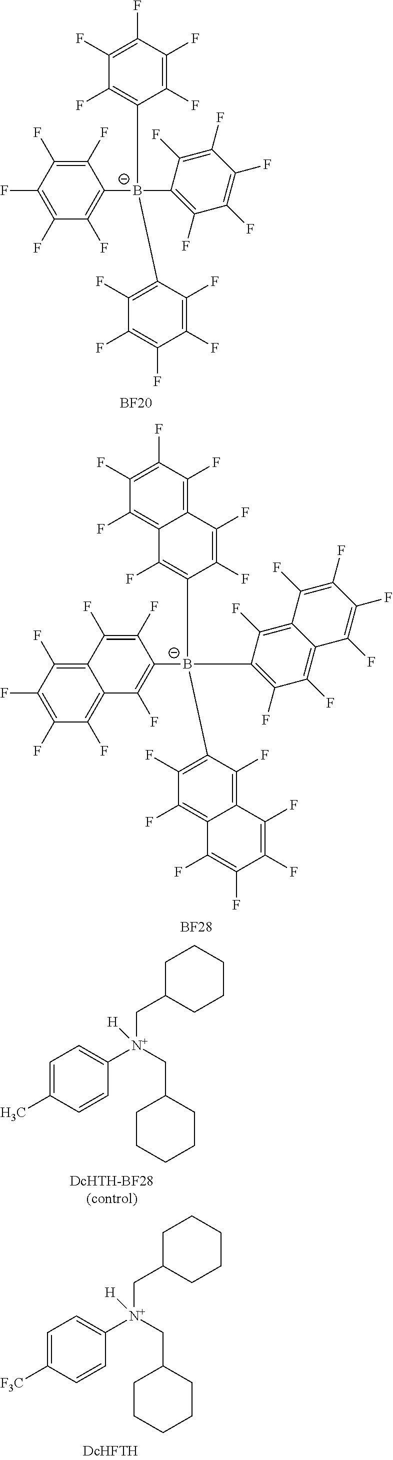

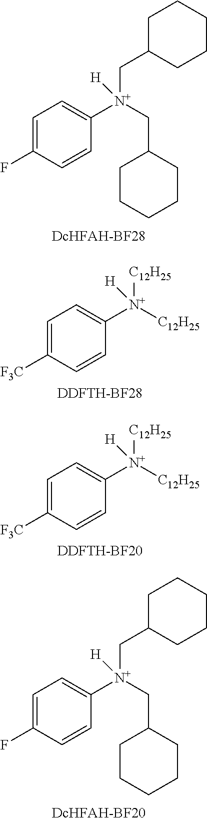

General Synthesis of Borate Activators:

Ammonium borate activators were prepared using a two-step process. In the first step, an amine was dissolved in a solvent (e.g. hexane, cyclohexane, methylcyclohexane, ether, dichloromethane, toluene) and an excess (ca. 1.2 molar equivalents) of hydrogen chloride was added to form an ammonium chloride salt. This salt was isolated by filtration from the reaction medium and dried under reduced pressure. The isolated ammonium chloride was then heated to reflux with one molar equivalent of an alkali metal borate in a solvent (e.g. cyclohexane, dichloromethane, methylcyclohexane) to form the ammonium borate along with byproduct alkali metal chloride, the latter which typically is removed by filtration. Examples describing the synthetic details for selected ammonium borates are given below.

### N,N-bis(cyclohexylmethyl)-4-trifluoromethylaniline (DcHFT)

4-(trifluoromethyl)aniline (1.0 g, 6.2 mmol) and cyclohexanecarbaldehyde (1.4 g, 12 mmol) were combined in 50 mL of dichloromethane. The sodium triacetoxyborohydride (2.9 g, 14 mmol) was added slowly, causing the reaction to bubble. After stirring at room temperature for 16 hours, the solution was diluted with saturated aqueous ammonium chloride and stirred for 30 minutes. The solution was brought to pH=10 with 1N NaOH solution, and extracted with 3 portions of dichloromethane. Combined organic fractions were washed with brine, then dried with MgSO₄, filtered and concentrated to a yellow oil. The dialkylated product was purified by silica gel chromatography using 20% acetone/isohexane as eluent. It was obtained as a white solid in 5% yield. ¹H NMR (500 MHz, CDCl₃, δ): 0.91 (m, 4H), 1.17 (m, 6H), 1.71 (m, 12H), 3.18 (d, J=6.9 Hz, 4H), 6.60 (d, J=8.8 Hz, 2H), 7.39 (d, J=8.7 Hz, 2H).

### N,N-bis(cyclohexylmethyl)-4-trifluoromethylbenzenaminium-BF28 (DcHFTH-BF28)

The above N,N-bis(cyclohexylmethyl)-4-trifluoromethylaniline (0.36 g, 1.0 mmol) was dissolved in 50 mL of hexane. A 2 M ethereal solution of HCl (0.51 mL, 1.0 mmol) was added slowly, causing a white precipitate to form. After stirring for 1 hour, the white solid was collected, washed with fresh hexane, and dried under vacuum to give the anilinium salt in 70% yield. A slurry of the aniline HCl salt (280 mg, 0.72 mmol) and Na—BF28 (750 mg, 0.72 mmol) was heated at reflux for 1.5 h in 50 mL cyclohexane. Once cooled to ambient, the mixture was filtered. The filtrate was concentrated, redissolved in dichloromethane, filtered through Celite, and then concentrated to give the product as a tan solid in 20% yield. ¹H NMR (400 MHz, CDCl₃, δ): 0.92 (m, 4H), 1.17 (m, 6H), 1.71 (m, 12H), 3.18 (d, J=6.9 Hz, 4H), 6.60 (d, J=8.8 Hz, 2H), 7.40 (d, J=8.8 Hz, 2H).

### N,N-bis(cyclohexylmethyl)-4-fluoroaniline (DcHFA)

4-fluoroaniline (2.0 g, 18 mmol) and cyclohexanecarbaldehyde (4.0 g, 36 mmol) were combined in 100 mL of dichloromethane. The sodium triacetoxyborohydride (8.4 g, 40 mmol) was added slowly, causing the reaction to bubble. After stirring at room temperature for 16 hours, the solution was diluted with saturated aqueous ammonium chloride and stirred for 30 minutes. The solution was brought to pH=10 with 1N NaOH solution, and extracted with 3 portions of dichloromethane. Combined organic fractions were washed with brine, then dried with MgSO₄, filtered and concentrated to a tan solid. The dialkylated product was purified by silica gel chromatography using 2% acetone/isohexane as eluent. It was obtained as a white solid in 57% yield. ¹H NMR (400 MHz, CDCl₃, δ): 0.88 (m, 4H), 1.15 (m, 6H), 1.70 (m, 12H), 3.08 (d, J=6.6 Hz, 4H), 6.53 (m, 2H), 6.89 (t, J=8.7 Hz, 2H).

### N,N-bis(cyclohexylmethyl)-4-fluorobenzenaminium-BF20 (DcHFAH-BF20)

The above N,N-bis(cyclohexylmethyl)-4-fluoroaniline (1.0 g, 3.3 mmol) was dissolved in 250 mL of hexane. A 2 M ethereal solution of HCl (1.6 mL, 3.3 mmol) was added slowly, causing a white precipitate to form. After stirring for 16 hours, the white solid was collected, washed with fresh hexane, and dried under vacuum to give the anilinium salt in 99% yield. A slurry of the aniline HCl salt (474 mg, 1.4 mmol) and Li—BF20 (1.1 g, 1.4 mmol) was heated at reflux for 1.5 h in 100 mL cyclohexane. Once cooled to ambient, the mixture was filtered. The filtrate was concentrated, redissolved in dichloromethane, filtered through Celite, and then concentrated to give the product as a white solid in 36% yield. ¹H NMR (500 MHz, CDCl₃, δ): 1.01 (m, 10H), 1.43 (m, 4H), 1.71 (m, 8H), 3.35 (m, 4H), 6.75 (br s, 1H), 7.31 (m, 4H).

### N,N-bis(cyclohexylmethyl)-4-fluorobenzenaminium-BF28 (DcHFAH-BF28)

A slurry of the above aniline HCl salt (500 mg, 1.5 mmol) and Na—BF28 (1.5 g, 1.5 mmol) was heated at reflux for 1.5 hours in 75 mL cyclohexane. Once cooled to ambient, the mixture was filtered. The filtrate was concentrated, redissolved in dichloromethane, filtered through Celite, and then concentrated to give the product as a brown oil in 4% yield. ¹H NMR (400 MHz, CDCl₃, δ): 0.88 (m, 4H), 1.16 (m, 6H), 1.69 (m, 12H), 3.08 (d, J=6.0 Hz, 4H), 6.53 (m, 2H), 6.90 (m, 2H).

### N,N-didodecyl-4-(trifluoromethyl)aniline (DDFT)

4-(trifluoromethyl)aniline (1.5 g, 9.3 mmol) and lauric aldehyde (3.4 g, 19 mmol) were combined in 100 mL of dichloromethane. The sodium triacetoxyborohydride (4.3 g, 20 mmol) was added slowly, causing the reaction to bubble. After stirring at room temperature for 16 hours, the solution was diluted with saturated aqueous ammonium chloride and stirred for 30 minutes. The solution was brought to pH=10 with 1N NaOH solution, and extracted with 3 portions of dichloromethane. Combined organic fractions were washed with brine, then dried with MgSO₄, filtered and concentrated to a yellow oil. The dialkylated product was purified by silica gel chromatography using 10% acetone/isohexane as eluent. It was obtained as a colorless oil in 22% yield. ¹H NMR (400 MHz, CDCl₃, δ): 0.88 (t, J=6.9 Hz, 6H), 1.26 (m, 40H), 3.27 (m, 4H), 6.60 (d, J=8.8 Hz, 2H), 7.40 (d, J=8.8 Hz, 2H).

### N,N-didodecyl-4-(trifluoromethyl)benzenaminium-BF20 (DDFTH-BF20)

The above N,N-didodecyl-4-(trifluoromethyl)aniline (1.0 g, 2.0 mmol) was dissolved in 100 mL of hexane. A 2 M ethereal solution of HCl (1.0 mL, 2.0 mmol) was added slowly, causing a white precipitate to form. After stirring for 1 hour, the white solid was collected, washed with fresh hexane, and dried under vacuum to give the anilinium salt in 77% yield. A slurry of the aniline HCl salt (384 mg, 0.72 mmol) and Li—BF20 (546 mg, 0.72 mmol) was heated at reflux for 1.5 h in 100 mL cyclohexane. Once cooled to ambient, the supernatant was decanted away from a yellow oil. The oil was dissolved in dichloromethane, filtered through Celite, and then concentrated to give the product as a yellow oil in 24% yield. ¹H NMR (500 MHz, CDCl₃, δ): 0.87 (t, J=6.9 Hz, 6H), 1.21 (m, 38H), 1.59 (m, 2H), 3.51 (m, 4H), 7.48 (m, 2H), 7.89 (m, 2H).

### N,N-didodecyl-4-(trifluoromethyl)benzenaminium-BF28 (DDFTH-BF28)

A slurry of the above aniline HCl salt (400 mg, 0.75 mmol) and Na—BF28 (783 mg, 0.75 mmol) was heated at reflux for 1.5 h in 75 mL cyclohexane. Once cooled to ambient, the mixture was filtered. The filtrate was concentrated, redissolved in dichloromethane, filtered through Celite, and then concentrated to give the product as a blue solid in 9% yield. ¹H NMR (400 MHz, CDCl₃, δ): 0.86 (t, J=6.9 Hz, 6H), 1.23 (m, 36H), 1.57 (m, 2H), 1.69 (m, 2H), 3.30 (m, 4H), 6.59 (m, 1H), 7.40 (m, 1H), 7.67 (m, 1H), 7.81 (m, 1H).

### Solubility Studies of the Activators

Procedure for Solid Activators.

A saturated solution of each of the solid ammonium borate activators was prepared by stirring an excess of the activator with either isohexane or methylcyclohexane solvent for several hours at 25° C. The mixture was then filtered and a known volume of the filtrate was evaporated to dryness in a tared vial. The vial was then weighed to determine the mass of activator that was dissolved in the aliquot. The concentration of the saturated solution is presented in millimoles of activator per liter of solution (mM).

Procedure for Activators that Form Emulsions.

A saturated solution of the activator was prepared by slowly adding the solvent to a pre-weighed amount of the activator. The final volume was determined as the minimum amount of solvent required to convert the emulsion into a homogeneous solution. The concentration of the saturated solution is presented in millimoles of activator per liter of solution (mM). Alternatively, an emulsion of the activator was prepared by adding the solvent to an excess amount of activator. The resulting emulsion was separated from the undissolved solids by decanting the emulsion into a tared vial. Solvent was then added dropwise until the emulsion became homogeneous. The mass of the final solution was measured and then the solvent was evaporated to dryness to obtain the mass of the activator. The concentration of the saturated solution is presented in millimoles of activator per liter of solution (mM).

### Polymerization in Parallel Pressure Reactor

Solvents, polymerization-grade toluene, and isohexane were supplied by ExxonMobil Chemical Company and purified by passing through a series of columns: two 500 cc Oxyclear cylinders in series from Labclear (Oakland, Calif.), followed by two 500 cc columns in series packed with dried 3 Å mole sieves (8-12 mesh; Aldrich Chemical Company), and two 500 cc columns in series packed with dried 5 Å mole sieves (8-12 mesh; Aldrich Chemical Company). 1-octene (C₈) and 1-hexene (C₆) (98%, Aldrich Chemical Company) were dried by stirring over NaK overnight followed by filtration through basic alumina (Aldrich Chemical Company, Brockman Basic 1).

Polymerization-grade ethylene (C2) was used and further purified by passing the gas through a series of columns: 500 cc Oxyclear cylinder from Labclear (Oakland, Calif.) followed by a 500 cc column packed with dried 3A mole sieves (8-12 mesh; Aldrich Chemical Company) and a 500 cc column packed with dried 5A mole sieves (8-12 mesh; Aldrich Chemical Company).

Polymerization grade propylene (C₃) was used and further purified by passing it through a series of columns: 2250 cc Oxiclear cylinder from Labclear followed by a 2250 cc column packed with 3 Å mole sieves (8-12 mesh; Aldrich Chemical Company), then two 500 cc columns in series packed with 5 Å mole sieves (8-12 mesh; Aldrich Chemical Company), then a 500 cc column packed with Selexsorb CD (BASF), and finally a 500 cc column packed with Selexsorb COS (BASF).

Solutions of the metal complexes and activators were prepared in a drybox using toluene or methylcyclohexane. Concentrations were typically 0.2 mmol/L. Tri-n-octylaluminum (TNOAL, neat, AkzoNobel) was typically used as a scavenger. Concentration of the TNOAL solution in toluene ranged from 0.5 to 2.0 mmol/L.

Polymerizations were carried out in a parallel pressure reactor, as generally described in U.S. Pat. Nos. 6,306,658; 6,455,316; 6,489,168; WO 00/09255; and Murphy, V. et al. (2003) “A Fully Integrated High-Throughput Screening Methodology for the Discovery of New Polyolefin Catalysts: Discovery of a New Class of High Temperature Single-Site Group (IV) Copolymerization Catalysts,” J. Am. Chem. Soc., v. 125(14), pp. 4306-4317, each of which is fully incorporated herein by reference. The experiments were conducted in an inert atmosphere (N₂) drybox using autoclaves equipped with an external heater for temperature control, glass inserts (internal volume of reactor=23.5 mL for C2 and C2/C8; 22.5 mL for C3 runs), septum inlets, regulated supply of nitrogen, ethylene and propylene, and equipped with disposable PEEK mechanical stirrers (800 RPM). The autoclaves were prepared by purging with dry nitrogen at 110° C. or 115° C. for 5 hours and then at 25° C. for 5 hours. Although the specific quantities, temperatures, solvents, reactants, reactant ratios, pressures, and other variables are frequently changed from one polymerization run to the next, the following describes a typical polymerization performed in a parallel pressure reactor.

Catalyst systems dissolved in solution were used in the polymerization examples below, unless specified otherwise.

A typical polymerization procedure is as follows:

### Ethylene-Octene Copolymerization (EO).

A pre-weighed glass vial insert and disposable stirring paddle are fitted to each reaction vessel of the reactor, which contains 48 individual reaction vessels. The reactor is then closed and purged with ethylene. Each vessel is charged with enough solvent (typically isohexane) to bring the total reaction volume, including the subsequent additions, to the desired volume, typically 5 mL. 1-octene, if required, is injected into the reaction vessel and the reactor is heated to the set temperature and pressurized to the predetermined pressure of ethylene, while stirring at 800 rpm. The aluminum compound in toluene is then injected as scavenger followed by addition of the activator solution (typically 1.0-1.2 molar equivalents).

The catalyst and activator solutions are all prepared in toluene. The catalyst solution (typically 0.020-0.080 μmol of metal complex) is injected into the reaction vessel and the polymerization is allowed to proceed until a pre-determined amount of ethylene (quench value typically 20 psi) had been used up by the reaction. Alternatively, the reaction may be allowed to proceed for a set amount of time (maximum reaction time typically 30 minutes). Ethylene is added continuously (through the use of computer controlled solenoid valves) to the autoclaves during polymerization to maintain reactor gauge pressure (P setpt, +/−2 psig) and the reactor temperature (T) is monitored and typically maintained within +/−1° C. The reaction is quenched by pressurizing the vessel with compressed air. After the reactor is vented and cooled, the glass vial insert containing the polymer product and solvent is removed from the pressure cell and the inert atmosphere glove box, and the volatile components are removed using a Genevac HT-12 centrifuge and Genevac VC3000D vacuum evaporator operating at elevated temperature and reduced pressure. The vial is then weighed to determine the yield of the polymer product. The resultant polymer is analyzed by Rapid GPC (see below) to determine the molecular weight, by FT-IR (see below) to determine percent octene incorporation, and by DSC (see below) to determine melting point (Tₘ).

Equivalence is determined based on the mole equivalents relative to the moles of the transition metal in the catalyst complex.

Propylene Homopolymerization (PP).

The reactor was prepared as described above and purged with propylene. Isohexane was then injected into each vessel at room temperature followed by a predetermined amount of propylene gas. The reactor was heated to the set temperature while stirring at 800 rpm, and the scavenger, activator and catalyst solutions were injected sequentially to each vessel. The polymerization was allowed to proceed as described previously.

Polymer Characterization.

Polymer sample solutions were prepared by dissolving polymer in 1,2,4-trichlorobenzene (TCB, 99+% purity from Sigma-Aldrich) containing 2,6-di-tert-butyl-4-methylphenol (BHT, 99% from Aldrich) at 165° C. in a shaker oven for approximately 3 hours. The typical concentration of polymer in solution was between 0.1 to 0.9 mg/mL with a BHT concentration of 1.25 mg BHT/mL of TCB.

To determine various molecular weight related values by GPC, high temperature size exclusion chromatography was performed using an automated “Rapid GPC” system as generally described in U.S. Pat. Nos. 6,491,816; 6,491,823; 6,475,391; 6,461,515; 6,436,292; 6,406,632; 6,175,409; 6,454,947; 6,260,407; and 6,294,388; each of which is fully incorporated herein by reference for US purposes. This apparatus has a series of three 30 cm×7.5 mm linear columns, each containing PLgel 10 m, Mix B. The GPC system was calibrated using polystyrene standards ranging from 580 to 3,390,000 g/mol. The system was operated at an eluent flow rate of 2.0 mL/minutes and an oven temperature of 165° C. 1,2,4-trichlorobenzene was used as the eluent. The polymer samples were dissolved in 1,2,4-trichlorobenzene at a concentration of 0.28 mg/mL and 400 uL of a polymer solution was injected into the system. The concentration of the polymer in the eluent was monitored using an evaporative light scattering detector. The molecular weights presented are relative to linear polystyrene standards and are uncorrected, unless indicated otherwise.

Differential Scanning Calorimetry (DSC) measurements were performed on a TA-Q100 instrument to determine the melting point (Tm) of the polymers. Samples were pre-annealed at 220° C. for 15 minutes and then allowed to cool to room temperature overnight. The samples were then heated to 220° C. at a rate of 100° C./min and then cooled at a rate of 50° C./min. Melting points were collected during the heating period.

The weight percent of ethylene incorporated in polymers was determined by rapid FT-IR spectroscopy on a Bruker Equinox 55+IR in reflection mode. Samples were prepared in a thin film format by evaporative deposition techniques. FT-IR methods were calibrated using a set of samples with a range of known wt % ethylene content. For ethylene-1-octene copolymers, the wt % octene in the copolymer was determined via measurement of the methyl deformation band at ˜1375 cm₋₁. The peak height of this band was normalized by the combination and overtone band at ˜4321 cm⁻¹, which corrects for path length differences.

A series of propylene polymerizations were performed in parallel pressure reactors (PPRs) developed by Symyx Technologies, Inc. In these polymerizations, the metallocene rac-dimethylsilyl-bis(indenyl)hafnium dimethyl (MCN-1) was used along with several different ammonium borate activators. Polymerizations were performed at 85° C., MCN-1 was present at 0.03 umol, activator was present at 1.2 equiv, propylene pressure was 135 psi, solvent was isohexane; total volume was 5 mL; tri(n-octyl)aluminum (TNOL) was present at 0.25, 0.50, or 0.75 umol. The data and run conditions are shown in Table 2.

TABLE 2
Data for the polymerization of propylene.

| | | | | | activity | | | | |
|---|---|---|---|---|---|---|---|---|---|
| | | TNOAL | rxn | | (g/mmol- | Mw | Mn | | |
| Ex. | activator | umol | time (s) | yield (g) | hr) | (kg/mol) | (kg/mol) | Mw/Mn | Tm |
| 1 | DcHFTH-BF28 | 0.25 | 1201 | −0.001 | −100 | | | | |
| 2 | DcHFTH-BF28 | 0.25 | 1202 | −0.001 | −100 | | | | |
| 3 | DcHFTH-BF28 | 0.25 | 1200 | −0.001 | −100 | | | | |
| 4 | DcHFTH-BF28 | 0.25 | 1201 | 0.001 | 100 | | | | |
| 5 | DDFTH-BF28 | 0.25 | 1201 | 0.001 | 100 | | | | |
| 6 | DDFTH-BF28 | 0.25 | 676 | 0.035 | 10,033 | 289 | 168 | 1.7 | 133.5 |
| 7 | DDFTH-BF28 | 0.25 | 1201 | 0.008 | 799 | | | | |
| 8 | DcHFAH-BF28 | 0.25 | 1201 | −0.001 | −100 | | | | |
| 9 | DcHFAH-BF28 | 0.25 | 1201 | −0.002 | −200 | | | | |
| 10 | DcHFAH-BF28 | 0.25 | 1201 | −0.001 | −100 | | | | |
| 11 | DcHTH-BF28 | 0.25 | 740 | 0.036 | 6,954 | 300 | 167 | 1.8 | 133.2 |
| 12 | DcHTH-BF28 | 0.25 | 978 | 0.033 | 5,456 | 289 | 181 | 1.6 | 134.0 |
| 13 | DcHTH-BF28 | 0.25 | 583 | 0.041 | 15,933 | 268 | 149 | 1.8 | 133.2 |
| 14 | DcHTH-BF28 | 0.25 | 1200 | 0.011 | 1,100 | 298 | 167 | 1.8 | 133.4 |
| 15 | DcHFTH-BF28 | 0.50 | 1201 | 0.003 | 300 | | | | |
| 16 | DcHFTH-BF28 | 0.50 | 1200 | −0.001 | −100 | | | | |
| 17 | DcHFTH-BF28 | 0.50 | 1200 | −0.001 | −100 | | | | |
| 18 | DDFTH-BF28 | 0.50 | 153 | 0.056 | 81,951 | 229 | 137 | 1.7 | 133.2 |
| 19 | DDFTH-BF28 | 0.50 | 235 | 0.080 | 96,096 | 265 | 166 | 1.6 | 133.7 |
| 20 | DDFTH-BF28 | 0.50 | 220 | 0.081 | 104,741 | 263 | 158 | 1.7 | 133.5 |
| 21 | DcHFAH-BF28 | 0.50 | 1201 | −0.001 | −100 | | | | |
| 22 | DcHFAH-BF28 | 0.50 | 1201 | −0.001 | −100 | | | | |
| 23 | DcHFAH-BF28 | 0.50 | 1200 | 0.001 | 100 | | | | |
| 24 | DcHTH-BF28 | 0.50 | 510 | 0.033 | 11,168 | 254 | 151 | 1.7 | 133.4 |
| 25 | DcHTH-BF28 | 0.50 | 510 | 0.043 | 16,283 | 314 | 183 | 1.7 | 134.8 |
| 26 | DcHTH-BF28 | 0.50 | 707 | 0.035 | 9,797 | 284 | 155 | 1.8 | 135.0 |
| 27 | DcHTH-BF28 | 0.50 | 710 | 0.033 | 8,436 | 271 | 155 | 1.7 | 134.0 |
| 28 | DcHFTH-BF28 | 0.75 | 1200 | 0.001 | 100 | | | | |
| 29 | DcHFTH-BF28 | 0.75 | 1201 | −0.001 | −100 | | | | |
| 30 | DcHFTH-BF28 | 0.75 | 1200 | −0.001 | −100 | | | | |
| 31 | DDFTH-BF28 | 0.75 | 204 | 0.076 | 95,597 | 245 | 147 | 1.7 | 134.0 |
| 32 | DDFTH-BF28 | 0.75 | 206 | 0.063 | 75,827 | 244 | 140 | 1.7 | 133.7 |
| 33 | DDFTH-BF28 | 0.75 | 193 | 0.068 | 90,166 | 244 | 138 | 1.8 | 133.9 |
| 34 | DDFTH-BF28 | 0.75 | 233 | 0.063 | | 249 | 153 | 1.6 | 133.5 |
| 35 | DcHFAH-BF28 | 0.75 | 1201 | −0.001 | −100 | | | | |
| 36 | DcHFAH-BF28 | 0.75 | 1200 | −0.001 | −100 | | | | |
| 37 | DcHFAH-BF28 | 0.75 | 1201 | −0.001 | −100 | | | | |
| 38 | DcHFAH-BF28 | 0.75 | 1201 | −0.001 | −100 | | | | |
| 39 | DcHTH-BF28 | 0.75 | 1201 | 0.008 | 799 | | | | |
| 40 | DcHTH-BF28 | 0.75 | 920 | 0.029 | 4,997 | 276 | 161 | 1.7 | 133.5 |
| 41 | DcHTH-BF28 | 0.75 | 1202 | 0.010 | 999 | 264 | 147 | 1.8 | 133.4 |

Overall, activators, catalyst systems, and methods of the present disclosure can provide improved solubility in aliphatic solvents, as compared to conventional activator compounds and catalyst systems. Activators, catalyst systems, and methods of the present disclosure can provide polyolefins having a weight average molecular weight (Mw) of about 1,000 g/mol or greater.

All documents described herein are incorporated by reference herein, including any priority documents and/or testing procedures to the extent they are not inconsistent with this text. As is apparent from the foregoing general description and the specific embodiments, while forms of the present disclosure have been illustrated and described, various modifications can be made without departing from the spirit and scope of the present disclosure. Accordingly, it is not intended that the present disclosure be limited thereby. Likewise, the term “comprising” is considered synonymous with the term “including” for purposes of United States law. Likewise whenever a composition, an element or a group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa.

What is claimed is:

1. A compound represented by Formula (AI):
[R¹R²R³EH]^{d+}[M^{k+}Qₙ]^{d-} (AI)
wherein: E is nitrogen or phosphorous (preferably nitrogen);
d is 1, 2 or 3; k is 1, 2, or 3; n is 1, 2, 3, 4, 5, or 6; n−k=d;
R¹ is electron deficient aromatic group;
each R², and R³ is independently C₁-C₄₀ linear alkyl or C₅-C₅₀-aryl, wherein each of R² and R³ is independently unsubstituted or substituted with at least one of halide, C₁-C₅₀ alkyl, C₅-C₅₀ aryl, C₆-C₃₅ arylalkyl, or C₆-C₃₅ alkylaryl, wherein R¹, R², and R³ together comprise 15 or more carbon atoms;
M is an element selected from group 13 of the Periodic Table of the Elements, preferably B or Al; and
each Q is independently a hydride, bridged or unbridged dialkylamido, halide, alkoxide, aryloxide, hydrocarbyl, substituted hydrocarbyl, halocarbyl, substituted halocarbyl, or halosubstituted-hydrocarbyl radical, provided that when each Q is perfluorophenyl, then R¹ is not methyl, and R² and R³ are not C₁₈ alkyl.

2. The compound of claim 1 wherein the compound is represented by Formula (I):
[R¹R²R³EH]⁺[BR⁴R⁵R⁶R⁷]⁻ (I)
wherein:
E is nitrogen or phosphorous (preferably nitrogen);
R¹ is electron deficient aromatic group,
each of R² and R³ is independently C₁-C₄₀ alkyl, C₅-C₂₂-aryl, wherein each of R¹, R², and R³ is independently unsubstituted or substituted with at least one of halide, C₁-C₁₀ alkyl, C₅-C₁₅ aryl, C₆-C₂₅ arylalkyl, and C₆-C₂₅ alkylaryl, wherein R¹, R², and R³ together comprise 15 or more carbon atoms;
each of R⁴, R⁵, R⁶, and R⁷ is aryl (such as phenyl or naphthyl), wherein at least one of R⁴, R⁵, R⁶, and R⁷ is substituted with from one or more fluorine atoms.

3. The compound of claim 2, wherein at least one of R⁴, R⁵, R⁶, and R⁷ is naphthyl substituted with seven fluorine atoms.

4. The compound of claim 2, wherein each of R⁴, R⁵, R⁶, and R⁷ is phenyl substituted with from one to five fluorine atoms.

5. The compound of claim 2, wherein each of R⁴, R⁵, R⁶, and R⁷ is naphthyl substituted with seven fluorine atoms.

6. The compound of claim 1, wherein R¹, R², and R³ together comprise 17 or more carbon atoms.

7. The compound of claim 1, wherein R¹, R², and R³ together comprise 20 or more carbon atoms.

8. The compound of claim 1, wherein R¹, R², and R³ together comprise 25 or more carbon atoms.

9. The compound of claim 1, wherein R¹, R², and R³ together comprise 35 or more carbon atoms.

10. The compound of claim 1, wherein R¹ is a phenyl group that is substituted with one, two, three, four or five halogen and/or haloalkyl groups and each of R² and R³ is independently C₁-C₄₀ linear alkyl, or C₅-C₂₂-aryl, wherein each of R² and R³ is independently unsubstituted or substituted with at least one C₁-C₁₀ alkyl.

11. The compound of claim 1, wherein R¹ is a phenyl group, that is substituted with one, two, three, four or five groups selected from fluoro, chloro, bromo, iodo, and trifluoromethyl and each of R² and R³ is independently selected from methyl, ethyl, n-propyl, n-butyl, n-pentyl, n-hexyl, n-heptyl, n-octyl, n-nonyl, n-decyl, n-undecyl, n-dodecyl, n-tridecyl, n-butadecyl, n-pentadecyl, n-hexadecyl, n-heptadecyl, n-octadecyl, n-nonadecyl, cyclohexylmethyl, and n-icosyl.

12. The compound of claim 1, wherein R¹ is selected from 4-fluorophenyl, 4-(trifluoromethyl)phenyl, 3-fluorophenyl, 3-(trifluoromethyl)phenyl, and 3-chlorophenyl.

13. The compound of claim 1, wherein R¹ is F₃C-phenyl or F-phenyl, R² is C₁₀-C₄₀ alkyl, and R³ is C₁₀-C₄₀ alkyl.

14. The compound of claim 1, wherein the cation, [R¹R²R³EH]⁺, is selected from the group consisting of:
N,N-didodecyl-2,3,4,5,6-pentafluorobenzenaminium,
N,N-didodecyl-3,5-difluorobenzenaminium,
N,N-didodecyl-3,5-bis(trifluoromethyl)benzenaminium,
N,N-bis(cyclohexylmethyl)-2,3,4,5,6-pentafluorobenzenaminium,
N,N-bis(cyclohexylmethyl)-3,5-bis(trifluoromethyl)benzenaminium,
N,N-bis(cyclohexylmethyl)-4-(trifluoromethyl)benzenaminium,
N,N-bis(cyclohexylmethyl)-4-fluorobenzenaminium, and
N,N-didodecyl-4-(trifluoromethyl)anilium.

15. The compound of claim 1, wherein the cation, [R¹R²R³EH]⁺, is selected from the group consisting of N,N-bis(cyclohexylmethyl)-4-(trifluoromethyl)benzenaminium, N,N-bis(cyclohexylmethyl)-4-fluorobenzenaminium, and N,N-didodecyl-4-(trifluoromethyl)anilium.

16. A catalyst system comprising a catalyst and the activator compound of claim 1.

17. The catalyst system of claim 16, further comprising a support material.

18. The catalyst system of claim 17, wherein the support material is selected from Al₂O₃, ZrO₂, SiO₂, SiO₂/Al₂O₃, SiO₂/TiO₂, silica clay, silicon oxide/clay, or mixtures thereof.

19. The catalyst system of claim 16, wherein the catalyst is represented by formula (II) or formula (III): 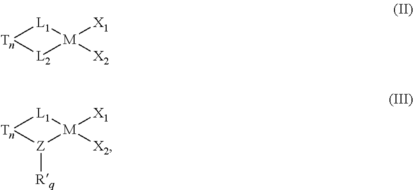
wherein in each of formula (II) and formula (III):
M is the metal center, and is a Group 4 metal;
n is 0 or 1;
T is an optional bridging group selected from dialkylsilyl, diarylsilyl, dialkylmethyl, ethylenyl or hydrocarbylethylenyl wherein one, two, three or four of the hydrogen atoms in ethylenyl are substituted by hydrocarbyl;
Z is nitrogen, sulfur, oxygen or phosphorus;
q is 1 or 2;
R′ is a C₁-C₄₀ alkyl or substituted alkyl group, preferably a linear C₁-C₄₀ alkyl or substituted alkyl group;
X₁ and X₂ are, independently, hydrogen, halogen, hydride radicals, hydrocarbyl radicals, substituted hydrocarbyl radicals, halocarbyl radicals, substituted halocarbyl radicals, silylcarbyl radicals, substituted silylcarbyl radicals, germylcarbyl radicals, or substituted germylcarbyl radicals; or both X₁ and X₂ are joined and bound to the metal atom to form a metallacycle ring containing from about 3 to about 20 carbon atoms; or both together can be an olefin, diolefin or aryne ligand.

20. The catalyst system of claim 16, wherein the catalyst is C₂ symmetrical.

21. The catalyst system of claim 16, wherein the catalyst is rac-dimethylsilyl-bis(indenyl)hafnium dimethyl.

22. The catalyst system of claim 16, wherein the catalyst is one or more of:
bis(1-methyl, 3-n-butyl cyclopentadienyl) M(R)₂;
dimethylsilyl bis(indenyl)M(R)₂;
bis(indenyl)M(R)₂;
dimethylsilyl bis(tetrahydroindenyl)M(R)₂;
bis(n-propylcyclopentadienyl)M(R)₂;
dimethylsilyl (tetramethylcyclopentadienyl)(cyclododecylamido)M(R)₂;
dimethylsilyl (tetramethylcyclopentadienyl)(cyclododecylamido)M(R)₂;
dimethylsilyl (tetramethylcyclopentadienyl)(t-butylamido)M(R)₂;
dimethylsilyl (tetramethylcyclopentadienyl)(t-butylamido)M(R)₂;
μ-(CH₃)₂Si(cyclopentadienyl)(1-adamantylamido)M(R)₂;
μ-(CH₃)₂Si(3-tertbutylcyclopentadienyl)(1-adamantylamido)M(R)₂;
μ-(CH₃)₂(tetramethylcyclopentadienyl)(1-adamantylamido)M(R)₂;
μ-(CH₃)₂Si(tetramethylcyclopentadienyl)(1-adamantylamido)M(R)₂;
μ-(CH₃)₂C(tetramethylcyclopentadienyl)(1-adamantylamido)M(R)₂;
μ-(CH₃)₂Si(tetramethylcyclopentadienyl)(1-tertbutylamido)M(R)₂;
μ-(CH₃)₂Si(fluorenyl)(1-tertbutylamido)M(R)₂;
μ-(CH₃)₂Si(tetramethylcyclopentadienyl)(1-cyclododecylamido)M(R)₂;
μ-(C₆H₅)₂C(tetramethylcyclopentadienyl)(1-cyclododecylamido)M(R)₂;
μ-(CH₃)₂Si(15-2,6,6-trimethyl-1,5,6,7-tetrahydro-s-indacen-1-yl)(tertbutylamido)M(R)₂;
where M is selected from Ti, Zr, and Hf; and R is selected from halogen or C₁ to C₅ alkyl.

23. The catalyst system of claim 16, wherein the catalyst is represented by Formula (BI), Formula (BII), or Formula (BIII): 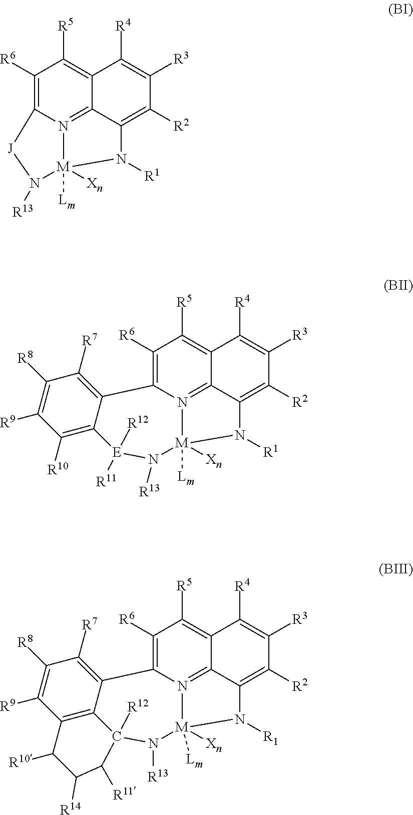
wherein:
M is a group 3, 4, 5, 6, 7, 8, 9, 10, 11, or 12 metal;
J is group including a three-atom-length bridge between the quinoline and the amido nitrogen;
E is carbon, silicon, or germanium;
X is an anionic leaving group;
L is a neutral Lewis base;
R¹ and R¹³ are independently selected from the group including of hydrocarbyls, substituted hydrocarbyls, and silyl groups;
R², R³, R⁴, R⁵, R⁶, R⁷, R⁸, R⁹, R¹⁰, R^{10′}, R¹¹, R^{11′}, R¹², and R¹⁴ are independently hydrogen, hydrocarbyl, alkoxy, silyl, amino, aryloxy, substituted hydrocarbyl, halogen, or phosphino;
n is 1 or 2;
m is 0, 1, or 2, where n+m is not greater than 4; and
any two R groups are optionally joined to form a substituted hydrocarbyl, unsubstituted hydrocarbyl, substituted heterocyclic, or unsubstituted heterocyclic, saturated or unsaturated ring, where the ring has 5, 6, 7, or 8 ring atoms and where substitutions on the ring can join to form additional rings;
any two X groups are optionally joined together to form a dianionic group;
any two L groups are optionally joined together to form a bidentate Lewis base; and
any X group is optionally joined to an L group to form a monoanionic bidentate group.

24. The catalyst system of claim 16, wherein the catalyst is represented by Formula (CI): 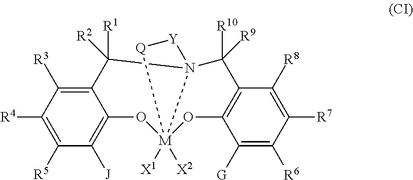
wherein M is a Group 4 metal; X¹ and X² are independently a univalent C₁-C₂₀ hydrocarbyl, C₁-C₂₀ substituted hydrocarbyl, a heteroatom or a heteroatom-containing group, or X¹ and X² join together to form a C₄-C₆₂ cyclic or polycyclic ring structure; each R¹, R², R³, R⁴, R⁵, R⁶, R⁷, R⁸, R⁹, and R¹⁰ is independently hydrogen, C₁-C₄₀ hydrocarbyl, C₁-C₄₀ substituted hydrocarbyl, a heteroatom or a heteroatom-containing group, or two or more of R¹, R², R³, R⁴, R⁵, R⁶, R⁷, R⁸, R⁹, or R¹⁰ are optionally joined together to form a C₄-C₆₂ cyclic or polycyclic ring structure, or a combination thereof; Q is a neutral donor group; J is heterocycle, a substituted or unsubstituted C₇-C₆₀ fused polycyclic group, where at least one ring is aromatic and where at least one ring, which may or may not be aromatic, has at least five ring atoms' G is as defined for J or may be hydrogen, C₂-C₆₀ hydrocarbyl, C₁-C₆₀ substituted hydrocarbyl, or optionally independently form a C₄-C₆₀ cyclic or polycyclic ring structure with R⁶, R⁷, or R⁸ or a combination thereof; Y is divalent C₁-C₂₀ hydrocarbyl or divalent C₁-C₂₀ substituted hydrocarbyl or (-Q-Y—) together form a heterocycle; and heterocycle may be aromatic and/or may have multiple fused rings.

25. The catalyst system of claim 16, wherein the catalyst is represented by Formula (IV): 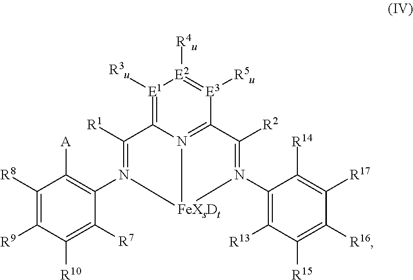
wherein:
A is chlorine, bromine, iodine, —CF₃ or —OR¹¹;
each of R¹ and R² is independently hydrogen, C₁-C₂₂-alkyl, C₂-C₂₂-alkenyl, C₆-C₂₂-aryl, arylalkyl where alkyl has from 1 to 10 carbon atoms and aryl has from 6 to 20 carbon atoms, or five-, six- or seven-membered heterocyclyl comprising at least one atom selected from the group consisting of N, P, O and S;
wherein each of R¹ and R² is optionally substituted by halogen, —NR¹¹₂, —OR¹¹ or —SiR¹²₃;
wherein R¹ optionally bonds with R³, and R² optionally bonds with R⁵, in each case to independently form a five-, six- or seven-membered ring;
R⁷ is a C₁-C₂₀ alkyl;
each of R³, R⁴, R⁵, R⁸, R⁹, R¹⁰, R¹⁵, R¹⁶, and R¹⁷ is independently hydrogen, C₁-C₂₂-alkyl, C₂-C₂₂-alkenyl, C₆-C₂₂-aryl, arylalkyl where alkyl has from 1 to 10 carbon atoms and aryl has from 6 to 20 carbon atoms, —NR¹¹₂, —OR¹¹, halogen, —SiR¹²₃ or five-, six- or seven-membered heterocyclyl comprising at least one atom selected from the group consisting of N, P, O, and S;
wherein R³, R⁴, R⁵, R⁷, R⁸, R⁹, R¹⁰, R¹⁵, R¹⁶, and R¹⁷ are optionally substituted by halogen, —NR¹¹₂, —OR¹¹ or —SiR¹²₃;
wherein R³ optionally bonds with R⁴, R⁴ optionally bonds with R⁵, R⁷ optionally bonds with R¹, R¹⁰ optionally bonds with R⁹, R⁹ optionally bonds with R⁸, R¹⁷ optionally bonds with R¹⁶, and R¹⁶ optionally bonds with R¹⁵, in each case to independently form a five-, six- or seven-membered carbocyclic or heterocyclic ring, the heterocyclic ring comprising at least one atom from the group consisting of N, P, O and S;
R¹³ is C₁-C₂₀-alkyl bonded with the aryl ring via a primary or secondary carbon atom;
R¹⁴ is chlorine, bromine, iodine, —CF₃ or —OR¹¹, or C₁-C₂₀-alkyl bonded with the aryl ring;
each R¹¹ is independently hydrogen, C₁-C₂₂-alkyl, C₂-C₂₂-alkenyl, C₆-C₂₂-aryl, arylalkyl where alkyl has from 1 to 10 carbon atoms and aryl has from 6 to 20 carbon atoms, or —SiR¹²₃, wherein R¹¹ is optionally substituted by halogen, or two R¹¹ radicals optionally bond to form a five- or six-membered ring;
each R¹² is independently hydrogen, C₁-C₂₂-alkyl, C₂-C₂₂-alkenyl, C₆-C₂₂-aryl, arylalkyl where alkyl has from 1 to 10 carbon atoms and aryl has from 6 to 20 carbon atoms, or two R¹² radicals optionally bond to form a five- or six-membered ring;
each of E¹, E², and E³ is independently carbon, nitrogen or phosphorus;
each u is independently 0 if E¹, E², and E³ is nitrogen or phosphorus and is 1 if E¹, E², and E³ is carbon;
each X is independently fluorine, chlorine, bromine, iodine, hydrogen, C₁-C₂₀-alkyl, C₂-C₁₀-alkenyl, C₆-C₂₀-aryl, arylalkyl where alkyl has from 1 to 10 carbon atoms and aryl has from 6 to 20 carbon atoms, —NR¹⁸₂, —OR¹⁸, —SR¹⁸, —SO₃R¹⁸, —OC(O)R¹⁸, —CN, —SCN, β-diketonate, —CO, —BF₄⁻, —PF₆⁻ or bulky non-coordinating anions, and the radicals X can be bonded with one another;
each R¹⁸ is independently hydrogen, C₁-C₂₀-alkyl, C₂-C₂₀-alkenyl, C₆-C₂₀-aryl, arylalkyl where alkyl has from 1 to 10 carbon atoms and aryl has from 6 to 20 carbon atoms, or —SiR¹⁹3, wherein R¹⁸ can be substituted by halogen or nitrogen- or oxygen-containing groups and two R¹⁸ radicals optionally bond to form a five- or six-membered ring;
each R¹⁹ is independently hydrogen, C₁-C₂₀-alkyl, C₂-C₂₀-alkenyl, C₆-C₂₀-aryl or arylalkyl where alkyl has from 1 to 10 carbon atoms and aryl has from 6 to 20 carbon atoms, wherein
R¹⁹ can be substituted by halogen or nitrogen- or oxygen-containing groups or two R¹⁹ radicals optionally bond to form a five- or six-membered ring;
s is 1, 2, or 3;
D is a neutral donor; and
t is 0 to 2.

26. The catalyst system of claim 16, wherein the catalyst is represented by Formula (VII): 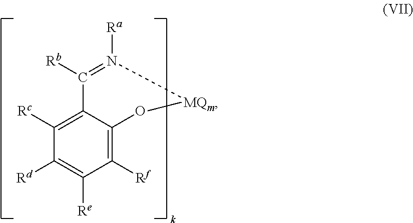
wherein M represents a transition metal atom selected from the groups 3 to 11 metals in the periodic table; k is an integer of 1 to 6; m is an integer of 1 to 6; R^{a} to R^{f} may be the same or different from one another and each represent a hydrogen atom, a halogen atom, a hydrocarbon group, a heterocyclic compound residue, an oxygen-containing group, a nitrogen-containing group, a boron-containing group, a sulfur-containing group, a phosphorus-containing group, a silicon-containing group, a germanium-containing group or a tin-containing group, among which 2 or more groups are optionally bound to each other to form a ring; when k is 2 or more, R^{a} groups, R^{b} groups, R^{c} groups, R^{d} groups, R^{e} groups, or R^{f} groups may be the same or different from one another, one group of R^{a} to R^{f} contained in one ligand and one group of R^{a} to R^{f} contained in another ligand may form a linking group or a single bond, and a heteroatom contained in R^{a} to R^{f} may coordinate with or bind to M; m is a number satisfying the valence of M; Q represents a hydrogen atom, a halogen atom, an oxygen atom, a hydrocarbon group, an oxygen-containing group, a sulfur-containing group, a nitrogen-containing group, a boron-containing group, an aluminum-containing group, a phosphorus-containing group, a halogen-containing group, a heterocyclic compound residue, a silicon-containing group, a germanium-containing group or a tin-containing group; when m is 2 or more, a plurality of groups represented by Q may be the same or different from one another, and a plurality of groups represented by Q may be mutually bound to form a ring.

27. The catalyst system of claim 16, wherein the catalyst is represented by Formula (VIII): 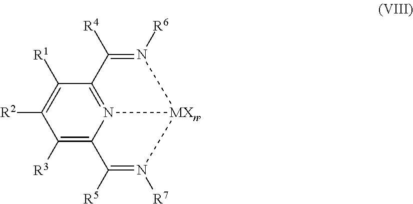
wherein:
M is Co or Fe; each X is an anion; n is 1, 2 or 3, so that the total number of negative charges on said anion or anions is equal to the oxidation state of a Fe or Co atom present in (VIII);
R¹, R² and R³ are each independently hydrogen, hydrocarbyl, substituted hydrocarbyl, or an inert functional group;
R⁴ and R⁵ are each independently hydrogen, hydrocarbyl, an inert functional group or substituted hydrocarbyl;
R⁶ is represented by the formula IX: 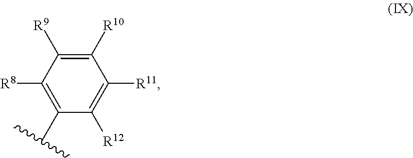
and R⁷ is represented by the formula X: 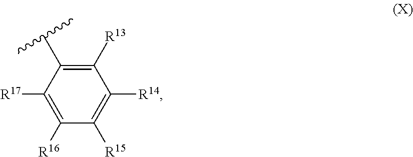
wherein R⁸ and R¹³ are each independently hydrocarbyl, substituted hydrocarbyl or an inert functional group;
R⁹, R¹⁰, R¹¹, R¹⁴, R¹⁵ and R¹⁶ are each independently hydrogen, hydrocarbyl, substituted hydrocarbyl or an inert functional group;
R¹² and R¹⁷ are each independently hydrogen, hydrocarbyl, substituted hydrocarbyl or an inert functional group;
and provided that any two of R⁸, R⁹, R¹⁰, R¹¹, R¹², R¹³, R¹⁴, R¹⁵, R¹⁶ and R¹⁷ that are adjacent to one another, together optionally form a ring.

28. The catalyst system of claim 16, wherein the catalyst is represented by Formula (XI): 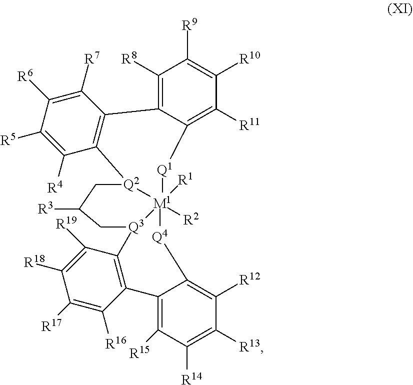
M¹ is selected from the group consisting of titanium, zirconium, hafnium, vanadium, niobium, tantalum, chromium, molybdenum and tungsten; each of Q¹, Q², Q³, and Q⁴ is independently oxygen or sulfur; R¹ and R² are independently hydrogen, halogen, hydroxyl, hydrocarbyl, or substituted hydrocarbyl, optionally R¹ and R² may also be joined together to form an alkanediyl group or a conjugated C₄-C₄₀ diene ligand which is coordinated to M¹;
each of R⁴, R⁵, R⁶, R⁷, R⁸, R⁹, R¹⁰, R¹¹, R¹², R¹³, R¹⁴, R¹⁵, R¹⁶, R¹⁷, R¹⁸, and R¹⁹ is independently hydrogen, halogen, C₁-C₄₀ hydrocarbyl or C₁-C₄₀ substituted hydrocarbyl, —NR′₂, —SR′, —OR, —OSiR′₃, —PR′₂, where each R′ is hydrogen, halogen, C₁-C₁₀ alkyl, or C₆-C₁₀ aryl, or one or more of R⁴ and R⁵, R⁵ and R⁶, R⁶ and R⁷, R⁸ and R⁹, R⁹ and R¹⁰, R¹⁰ and R¹¹, R¹² and R¹³, R¹³ and R¹⁴, R¹⁴ and R¹⁵, R¹⁶ and R¹⁷, R¹⁷ and R¹⁸, and R¹⁸ and R¹⁹ are optionally joined to form a saturated ring, unsaturated ring, substituted saturated ring, or substituted unsaturated ring;
R³ is a C₁-C₄₀ unsaturated alkyl or substituted C₁-C₄₀ unsaturated alkyl.

29. The catalyst system of claim 16, wherein the catalyst is represented by Formula (XII) or (XIII): 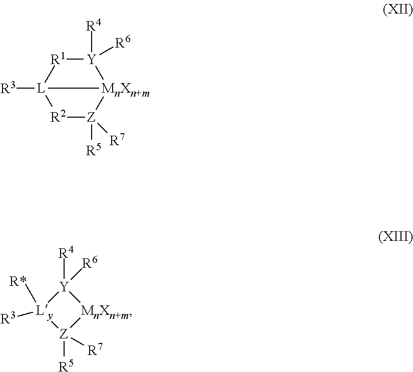
wherein M is a Group 3 to 12 transition metal or a Group 13 or 14 main group metal; each X is independently a leaving group; y is 0 or 1 (when y is 0 group L′ is absent); ‘n’ is the oxidation state of M and is +3, +4, or +5; ‘m’ represents the formal charge of the YZL or the YZL′ ligand, and is 0, −1, −2 or −3; L is a Group 15 or 16 element; L′ is a Group 15 or 16 element or Group 14 containing group; Y is a Group 15 element; Z is a Group 15 element; R¹ and R² are, independently, a C₁ to C₂₀ hydrocarbon group, a heteroatom containing group having up to twenty carbon atoms, silicon, germanium, tin, lead, or phosphorus; R³ is optionally absent or may be a hydrocarbon group, a hydrogen, a halogen, a heteroatom containing group; R⁴ and R⁵ are independently an alkyl group, an aryl group, substituted aryl group, a cyclic alkyl group, a substituted cyclic alkyl group, a cyclic aralkyl group, a substituted cyclic aralkyl group, or multiple ring system; R⁶ and R⁷ are independently absent, hydrogen, an alkyl group, halogen, heteroatom, or a hydrocarbyl group; R* may be absent, or may be a hydrogen, a Group 14 atom containing group, a halogen, or a heteroatom containing group.

30. A method of polymerizing olefins to produce at least one polyolefin, the method comprising contacting at least one olefin with the catalyst system of claim 16 and obtaining a polyolefin.

31. The method of claim 30, wherein the at least one olefin is propylene and the polyolefin is isotactic polypropylene.

32. A method of polymerizing olefins to produce at least one polyolefin, the method comprising contacting two or more different olefins with the catalyst system claim 16 and obtaining a polyolefin.

33. The method of claim 32, wherein the two or more olefins are ethylene and propylene.

34. The method of claim 32, wherein the two or more olefins further comprise a diene.

35. The method of claim 32, wherein the polyolefin has an Mw of from about 50,000 to about 300,000 g/mol and a melt temperature of from about 120° C. to about 140° C.

36. The method of claim 32, wherein the polyolefin has an Mw of from about 100,000 to about 300,000 g/mol and a melt temperature of from about 125° C. to about 135° C.

37. The method of claim 30, wherein the method is performed in gas phase or slurry phase.

38. The method of claim 30, wherein the method is performed in solution phase.

39. A solution comprising the compound of claim 1, and an aliphatic solvent.

40. The solution of claim 39, where aromatic solvents, such as toluene, are absent.

41. A solution comprising the catalyst system of claim 16, and an aliphatic solvent where, optionally, aromatic solvents are absent.

42. A composition comprising the catalyst system of claim 23, and an aliphatic solvent, where aromatic solvents are absent.
